(19) United States
(12) Patent Application Publication
(10) Pub. No.: US 2004/0023972 A1
(43) Pub. Date: Feb. 5, 2004

(54) Use of substituted imidazo[1,2-a]-pyridin-, -pyrimidin-and-pyrazin-3-yl-amine derivatives in the preparation of medicaments for inhibiting NOS

(75) Inventors: Bernd Sundermann, Aachen (DE); Corinna Maul, Aachen (DE); Hagen-Heinrich Hennies, Simmerath (DE); Johannes Schneider, Stolberg (DE)

Correspondence Address: CROWELL & MORING LLP, INTELLECTUAL PROPERTY GROUP, P.O. BOX 14300, WASHINGTON, DC, 20044-4300, US

(73) Assignee: Gruenenthal GmbH, Aachen (DE)

(21) Appl. No.: 10/411,402

(22) Filed: Apr. 11, 2003

Related U.S. Application Data
(63) Continuation of application No. PCT/EP01/11701, filed on Oct. 10, 2001.

(30) Foreign Application Priority Data
Oct. 13, 2000 (DE) 100 50 663.1

(51) Int. Cl. A61K 31/519; A61K 31/498; C07D 487/02

(52) U.S. Cl. 514/249; 514/259.1; 544/281; 544/350

(57) ABSTRACT

A pharmaceutical composition for the treatment of pain, migraine, multiple sclerosis, Parkinson's disease, Alzheimer's disease, Huntington's disease, cerebral ischaemia, diabetes, meningitis, or arteriosclerosis, or a method for healing wounds, the method comprising administering to a patient in need thereof an effective amount of a pharmaceutical composition comprising a compound of formula I

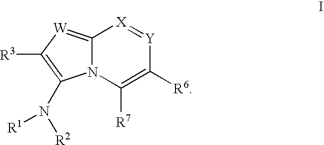

Also disclosed are methods of preparing the compounds of formula I, and treatment methods using the pharmaceutical compositions.

### CROSS REFERENCE TO RELATED APPLICATIONS

The present application is a continuation of International Patent Application No. PCT/EP01/11701, filed Oct. 10, 2001, designating the United States of America and published in German as WO 02/30428 A1, the entire disclosure of which is incorporated herein by reference. Priority is claimed based on Federal Republic of Germany Patent Application No. 100 50 663.1, filed Oct. 13, 2000.

### FIELD OF THE INVENTION

The invention relates to the use of substituted imidazo[1,2-a]-pyridin-, -pyrimidin- and -pyrazin-3-yl-amine derivatives in the preparation of medicaments for inhibiting NOS, in the preparation of medicaments for the treatment of migraine and in the preparation of medicaments for the treatment of septic shock, multiple sclerosis, Parkinson's disease, Alzheimer's disease, Huntington's disease, inflammations, inflammatory pain, cerebral ischemia, diabetes, meningitis, arteriosclerosis and/or for healing wounds.

### BACKGROUND OF THE INVENTION

Nitric oxide (NO) regulates a large number of physiological processes, including neurotransmission, the relaxation and proliferation of smooth muscle, the adhesion and aggregation of thrombocytes, and also tissue damage and inflammation. Owing to the large number of signal functions, NO is associated with a number of diseases (see, for example, L. J. Ignarro, Angew. Chem. (1999), 111, 2002-2013 and F. Murad, Angew. Chem. Int. Ed. (1999), 111, 1976-1989). The enzyme responsible for the physiological formation of NO, NO synthase (NOS), plays an important part in influencing those diseases therapeutically. Hitherto, three different isoforms of NO synthase have been identified, namely the two constitutive forms nNOS and eNOS and the inducible form iNOS (see A. J. Hobbs, A. Higgs, S. Moncada, Annu. Rev. Pharmacol. Toxicol. (1999), 39, 191-220; I. C. Green, P.-E. Chabrier, DDT (1999), 4, 47-49; P.-E. Chabrier et al., Cell. Mol. Life Sci. (1999), 55, 1029-1035).

The inhibition of NO synthase opens up new approaches to the therapy of various diseases associated with NO (A. J. Hobbs et al., Annu. Rev. Pharmacol. Toxicol. (1999), 39, 191220; I. C. Green, P.-E. Chabrier, DDT (1999), 4, 47-49; P.-E. Chabrier et al., Cell. Mol. Life Sci. (1999), 55, 1029-1035), such as migraine (L. L. Thomsen, J. Olesen, Clinical Neuroscience (1998), 5, 28-33; L. H. Lassen et al., The Lancet (1997), 349, 401-402), septic shock, neurodegenerative diseases such as multiple sclerosis, Parkinson's disease, Alzheimer's disease or Huntington's disease, inflammations, inflammatory pain, cerebral ischemia, diabetes, meningitis and arteriosclerosis. NOS inhibition can also have an effect on the healing of wounds, on tumors and on angiogenesis, and can also effect non-specific immunity to microorganisms (A. J. Hobbs et al., Annu. Rev. Pharmacol. Toxicol. (1999), 39, 191-220).

In addition to N-monₒ-methyl-L-arginine (L-NMMA) and nitro-L-arginine methyl ester (L-NAME), i.e. analogues of L-arginine from which NO and citrulline are formed in vivo with the involvement of NOS, NO-synthase-inhibiting active ingredients known hitherto also include S-methyl-L-citrulline, aminoguanidine, S-methylisourea, 7-nitroindazole and 2-mercaptO-ethylguanidine (A. J. Hobbs et al., Annu. Rev. Pharmacol. Toxicol. (1999), 39, 191-220).

By contrast, the object underlying the present invention was to provide novel effective NOS inhibitors.

### DESCRIPTION OF THE INVENTION

Surprisingly, it has been found that substituted imidazo[1,2-a]-pyridin-, -pyrimidin- and -pyrazin-3-yl-amine derivatives having the general formula I

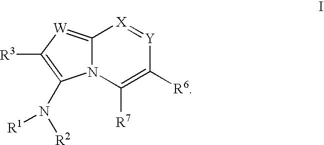

wherein

x represents CR⁴ or N,

Y represents CR⁵ or N and

X and Y do not simultaneously represent N,

W represents N or NR⁸,

R¹ represents C₁₋₁₂-alkyl, which is straight-chain or branched and is saturated or unsaturated and is unsubstituted or mono- or poly-substituted; C₃₋₈-cycloalkyl or CH₂—C₃₋₈-cycloalkyl, which is saturated or unsaturated and is unsubstituted or mono- or poly-substituted; heterocyclyl, which is saturated or unsaturated and is unsubstituted or mono- or poly-substituted; aryl, which is unsubstituted or mono- or poly-substituted; heteroaryl, which is unsubstituted or mono- or poly-substituted; C₁₋₈-alkyl-aryl or C₁₋₈-alkyl-heteroaryl, wherein the alkyl is straight-chain or branched and is saturated or unsaturated and is unsubstituted or mono- or poly-substituted, the aryl is unsubstituted or mono- or poly-substituted, and the heteroaryl is unsubstituted or mono- or poly-substituted,

R² represents hydrogen or C(═O)R⁹,

R³ represents C₁₋₈-alkyl, which is straight-chain or branched and is saturated or unsaturated and is unsubstituted or mono- or poly-substituted; C₃₋₈-cycloalkyl, which is saturated or unsaturated and is unsubstituted or mono- or poly-substituted;

heterocyclyl, which is saturated or unsaturated and is unsubstituted or mono- or poly-substituted; aryl, which is unsubstituted or mono- or poly-substituted; heteroaryl, which is unsubstituted or mono- or poly-substituted; C₁₋₈-alkyl-C₃₋₈-cycloalkyl, C₁₋₈alkyl-heterocyclyl, C₁₋₈-alkyl-aryl or C₁₋₈-alkyl-heteroaryl, wherein the alkyl is straight-chain or branched and is saturated or unsaturated and is unsubstituted or mono- or poly-substituted, the cycloalkyl is saturated or unsaturated and is unsubstituted or mono- or poly-substituted, the heterocyclyl is saturated or unsaturated and is unsubstituted or mono- or poly-substituted, the aryl is unsubstituted or mono- or poly-substituted, and the heteroaryl is unsubstituted or mono- or poly-substituted,

R⁴, R⁵, R⁶ and R⁷ each independently of the others represents hydrogen; Cl g-alkyl, which is straight-chain or branched and is saturated or unsaturated and is unsubstituted or mono- or poly-substituted, C₃₋₈-cycloalkyl or CH₂-C₃₋₈-cycloalkyl, wherein the cycloalkyl is saturated or unsaturated and is unsubstituted or mono- or poly-substituted; F; Cl; Br; I; CN; NO₂; NH₂; C(═O)R⁹; CO₂H; CO₂R¹⁰; OH or OR¹¹, or

R⁴ and R⁵, or R⁵ and R⁶, or R⁶ and R⁷ represent a four-membered saturated or unsaturated hydrocarbon bridge having zero, 1, 2 or 3 hetero atoms selected from N, O and S, and the other radicals of R⁴, R⁵, R⁶ and R⁷ represent hydrogen,

R⁸ represents C(═O)R⁹,

R⁹ represents C₁₋₈-alkyl, which is straight-chain or branched and is saturated or unsaturated and is unsubstituted or mono- or poly-substituted; C₃₋₈-cycloalkyl or CH₂—C₃₋₈-cycloalkyl, wherein the cycloalkyl is saturated or unsaturated and is unsubstituted or mono- or poly-substituted; heterocyclyl; which is saturated or unsaturated and is unsubstituted or mono- or poly-substituted; aryl, which is unsubstituted or mono- or poly-substituted; heteroaryl, which is unsubstituted or mono- or poly-substituted; C₁₋₈-alkyl-aryl or C₁₋₈-alkyl-heteroaryl, wherein the alkyl is straight-chain or branched and is saturated or unsaturated and is unsubstituted or mono- or poly-substituted, the aryl is unsubstituted or mono- or poly-substituted, and the heteroaryl is unsubstituted or mono- or poly-substituted, and

R¹⁰ and R¹¹ each independently of the other represents C₁₋₈-alkyl, which is straight-chain or branched and is saturated or unsaturated and is unsubstituted; or mono- or poly-substituted, C₃₋₈-cycloalkyl or CH₂—C₃₋₈-cycloalkyl, wherein the cycloalkyl is saturated or unsaturated and is unsubstituted or mono- or poly-substituted, aryl, which is unsubstituted or mono- or poly-substituted, C₁₋₈-alkyl-aryl, wherein the alkyl is straight-chain or branched and is saturated or unsaturated and is unsubstituted or mono- or poly-substituted, and the aryl is unsubstituted or mono- or poly-substituted,

are very effective NOS inhibitors.

The present invention accordingly relates to the use of the compounds having the general formula I as defined above, in the form of their bases or their pharmaceutically acceptable salts, in the preparation of a medicament for inhibiting NO synthase. The present invention relates also to the use of a compound having the general formula I, in the form of its base or of one of its pharmaceutically acceptable salts, in the preparation of a medicament for the treatment of migraine and for the treatment of septic shock, multiple sclerosis, Parkinson's disease, Alzheimer's disease, Huntington's disease, inflammations, inflammatory pain, cerebral ischemia, diabetes, meningitis, arteriosclerosis and/or for healing wounds.

It is preferred that, of the uses according to the invention, those compounds having the general formula I in which simultaneously R¹=tert-butyl, R²=H, X=CR⁴ wherein R⁴=H, Y=CR⁵ wherein R⁵=methyl, R⁶=H and R⁷=H or C₁₋₄-alkanyl (wherein alkanyl is straight-chain or branched and is unsubstituted or mono- or poly-substituted) are excluded.

Within the scope of this invention, the expressions “C₁₋₈-alkyl” and “C₁₋₁₂-alkyl” include acyclic saturated or unsaturated hydrocarbon radicals, which may be straight-chain or branched and may be unsubstituted or mono- or poly-substituted, having from 1 to 8 and from 1 to 12 carbon atoms, respectively, i.e. C₁₋₈-alkanyls, C₂₋₈-alkenyls and C₂₋₈-alkynyls, and C₁₋₁₂alkanyls, C₂₋₁₂-alkenyls and C₂₋₁₂-alkynyls, where alkenyls have at least one C—C double bond and alkynyls have at least one C—C triple bond. Alkyl is advantageously selected from the group comprising methyl, ethyl, n-propyl, 2-propyl, n-butyl, isobutyl, sec-butyl, tert-butyl, n-pentyl, isopentyl, neopentyl, n-hexyl, 2-hexyl, n-octyl, 1,1,3,3-tetramethylbutyl, n-decyl, n-dodecyl; ethylenyl (vinyl), ethynyl, propenyl (—CH₂CH═CH₂, —CH═CH—CH₃, —C(═CH₂)—CH₃), propynyl (—CH—C≡CH, —C≡C—CH₃), butenyl, butynyl, pentenyl, pentynyl, hexenyl, hexynyl, octenyl and octynyl.

For the purposes of this invention, the expression “C₃₋₈-cycloalkyl” means cyclic hydrocarbons having from 3 to 8 carbon atoms, which may be saturated or unsaturated, unsubstituted or mono- or poly-substituted. C₃₋₈-Cycloalkyl is advantageously selected from the group comprising cyclopropyl, cyclobutyl, cyclopentyl, cyclohexyl, cycloheptyl, cyclooctyl, cyclopentenyl, cyclohexenyl, cycloheptenyl and cyclooctenyl. Cycloalkyl is particularly preferably cyclohexyl.

The expression “heterocyclyl” denotes a 3-, 4-, 5-, 6- or 7-membered cyclic organic radical which contains at least 1, optionally also 2, 3, 4 or 5 hetero atoms, wherein the hetero atoms are identical or different and the cyclic radical is saturated or unsaturated, but is not aromatic, and may be unsubstituted or mono- or poly-substituted. The heterocycle may also be part of a bicyclic or polycyclic system. Preferred hetero atoms are nitrogen, oxygen and sulfur. It is preferred for the heterocyclyl radical to be selected from the group comprising tetrahydrofuryl, tetrahydropyranyl, pyrrolidinyl, piperidinyl, piperazinyl and morpholinyl, wherein the heterocyclyl radical may be bonded to the compound having the general formula I via any desired ring member.

Within the scope of this invention, the expression “aryl” means aromatic hydrocarbons, such as phenyls, naphthyls and phenanthrenyls. The aryl radicals may also be condensed with other saturated, (partially) unsaturated or aromatic ring systems. Each aryl radical may be unsubstituted or mono- or poly-substituted, it being possible for the aryl substituents to be identical or different and to be at any desired possible position of the aryl. Aryl is advantageously selected from the group containing phenyl, 1-naphthyl, 2-naphthyl and phenanthren-9-yl, each of which may be unsubstituted or mono- or poly-substituted.

The expression “heteroaryl” denotes a 5-, 6- or 7-membered cyclic aromatic radical which contains at least 1, optionally also 2, 3, 4 or 5 hetero atoms, the hetero atoms being identical or different and it being possible for the heterocycle to be unsubstituted or mono- or poly-substituted; in the case of substitution on the heterocycle, the heteroaryl substituents may be identical or different and may be at any desired possible position of the heteroaryl. The heterocycle may also be part of a bicyclic or polycyclic system. Preferred hetero atoms are nitrogen, oxygen and sulfur. It is preferred for the heteroaryl radical to be selected from the group containing pyrrolyl, indolyl, furyl (furanyl), benzofuranyl, thienyl (thiophenyl), benzothienyl, pyrazolyl, imidazolyl, thiazolyl, oxazolyl, isoxazolyl, pyridinyl, pyridazinyl, pyrimidinyl, pyrazinyl, pyranyl, indolyl, indazolyl, purinyl, pyrimidinyl, indolizinyl, quinolinyl, isoquinolinyl, quinazolinyl, carbazolyl, phenazinyl, phenothiazinyl, it being possible for the heteroaryl radical to be bonded to the compounds having the general formula I via any desired possible ring member. Particularly preferred heteroaryl radicals for the purposes of this invention are pyridin-2-yl, pyridin-3-yl, furan-2-yl, furan-3-yl, thien-2-yl (2-thiophene), thien-3-yl (3-thiophene) and benzo[b]furan-2-yl, each of which may be unsubstituted or mono- or poly-substituted.

For the purposes of the present invention, the expressions “C₁₋₈-alkyl-C₃₋₈-cycloalkyl” and “CH₂—C₃₋₈-cycloalkyl”, “C₁₋₈-alkyl-heterocyclyl”, “C₁₋₈-alkyl-aryl” or “C₁₋₈-alkyl-heteroaryl” mean that C₁₋₈-alkyl (or CH₂) and cycloalkyl, heterocyclyl, aryl and heteroaryl have the meanings defined above and the cycloalkyl, heterocyclyl, aryl or heteroaryl radical is bonded to the compound having the general formula I via a C₁₋₈-alkyl group (or in the case of “CH₂—C₃₋₈cycloalkyl” via a CH₂ group).

In connection with “alkyl”, “alkanyl”, “alkenyl” and “alkynyl”, the term “substituted” within the scope of this invention is understood to mean the substitution of a hydrogen radical by F, Cl, Br, I, —CN, —N≡C, NH₂, NH-alkyl, NH-aryl, NH-heteroaryl, NH-alkyl-aryl, NH-alkyl-heteroaryl, NH-heterocyclyl, NH-alkyl-OH, N(alkyl)₂, N(alkyl-aryl)₂, N(alkyl-heteroaryl)₂, N(heterocyclyl)₂, N(alkyl-OH)₂, NO, NO₂, SH, S-alkyl, S-aryl, S-heteroaryl, S-alkyl-aryl, S-alkyl-heteroaryl, S-heterocyclyl, S-alkyl-OH, S-alkyl-SH, OH, O-alkyl, O-aryl, O-heteroaryl, O-alkyl-aryl, O-alkyl-heteroaryl, O-heterocyclyl, O-alkyl-OH, CHO, C(═O)C₁₋₆-alkyl, C(═S)C₁₋₆-alkyl, C(═O)aryl, C(═S)aryl, C(═O)C₁₋₆-alkyl-aryl,

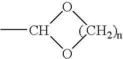

wherein n=1, 2 or 3, C(═S)C₁₋₆-alkyl-aryl, C(═O)-heteroaryl, C(═S)-heteroaryl, C(═O)-heterocyclyl, C(═S)-heterocyclyl, CO₂H, CO₂-alkyl, CO₂-alkyl-aryl, C(═O)NH₂, C(═O)NH-alkyl, C(═O)NH-aryl, C(═O)NH-heterocyclyl, C(═O)N(alkyl)₂, C(═O)N(alkyl-aryl)₂, C(═O)N(alkyl-heteroaryl)₂, C(═O)N(heterocyclyl)₂, SO-alkyl, SO₂-alkyl, SO₂NH₂, SO₃H, PO(O—C₁₋₆-alkyl)₂, Si(C₁₋₆-alkyl)₃, Si(C₃₋₈-cycloalkyl)₃, Si(CH₂—C₃₋₈-cycloalkyl)₃, Si(phenyl)₃, cycloalkyl, aryl, heteroaryl or by heterocyclyl, where poly-substituted radicals are understood to be radicals that are poly-substituted, for example di- or tri-substituted, either on different atoms or on the same atoms, for example trisubstituted on the same carbon atom, as in the case of CF₃ or —CH₂CF₃, or at different positions, as in the case of —CH(OH)—CH═CH—CHCl₂. Polysubstitution can be carried out with the same or with different substituents. It is also possible for a substituent itself to be substituted; accordingly, —O-alkyl also includes, inter alia, —O—CH₂—CH₂—O—CH₂—CH₂—OH.

For the purposes of the present invention, “alkyl” in this context particularly preferably means methyl, ethyl, CH₂—OH, CH₂CO₂H, CH₂CO₂-methyl, CH₂PO(O—C₁₋₆-alkanyl)₂, CH₂Si(C₁₋₆-alkanyl)₃, CH₂Si(C₃₋₈-cycloalkyl)₃, CH₂Si(CH₂—C₃₋₈-cycloalkyl)₃, CH₂Si(phenyl)₃, CH₂CH₂-morpholin-4-yl, CH₂-aryl, CF₃ or (CH₂)ₙ—N≡C wherein n=2, 3, 4, 5, or 6.

In relation to “aryl”, “heterocyclyl”, “heteroaryl” and “cycloalkyl”, “mono- or poly-substituted” within the scope of this invention is understood to mean the mono- or poly-substitution, for example di-, tri- or tetra-substitution, of one or more hydrogen atoms of the ring system by F, Cl, Br, I, CN, NH₂, NH-alkyl, NH-aryl, NH-heteroaryl, NH-alkyl-aryl, NH-alkyl-heteroaryl, —NH-heterocyclyl, NH-alkyl-OH, N(alkyl)₂, N(alkyl-aryl)₂, N(alkyl-heteroaryl)₂, N(heterocyclyl)₂, N(alkyl-OH)₂, NO, NO₂, SH, S-alkyl, S-cycloalkyl, S-aryl, S-heteroaryl, S-alkyl-aryl, S-alkyl-heteroaryl, S-heterocyclyl, S-alkyl-OH, S-alkyl-SH, OH, O-alkyl, O-cycloalkyl, O-aryl, O-heteroaryl, O-alkyl-aryl, O-alkyl-heteroaryl, O-heterocyclyl, O-alkyl-OH, CHO, C(═O)C₁₋₆-alkyl, C(═S)C₁₋₆-alkyl, C(═O)aryl, C(═S)aryl, C(═O)C₁₋₆-alkyl-aryl,

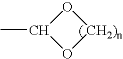

wherein n=1, 2 or 3, C(═S)C₁₋₆-alkyl-aryl, C(═O)-heteroaryl, C(═S)-heteroaryl, C(═O)-heterocyclyl, C(═S)-heterocyclyl, CO₂H, CO₂-alkyl, CO₂-alkyl-aryl, C(═O)NH₂, C(═O)NH-alkyl, C(═O)NH-aryl, C(═O)NH-heterocyclyl, C(═O)N(alkyl)₂, C(═O)N(alkyl-aryl)₂, C(═O)N(alkyl-heteroaryl)₂, C(═O)N(heterocyclyl)₂, S(O)-alkyl, S(O)-aryl, SO₂-alkyl, SO₂-aryl, SO₂NH₂, SO₃H, CF₃, ═O, ═S; alkyl, cycloalkyl, aryl, heteroaryl and/or by heterocyclyl; on one or, optionally, different atoms (it being possible for a substituent itself to be substituted). Polysubstitution is carried out with the same or with different substituents. Particularly preferred substituents for “aryl” are —F, —Cl, —Br, —CF₃, —OH, —O—CH₃, —O—CH₂CH₃, methyl, n-propyl, carboxy (—CO₂H), nitro, 4-chlorophenoxy, acetoxy and dimethylamino. Particularly preferred substituents for “heteroaryl” are methyl-OH, —O—CH₃, —CH₂OH, —NO₂, CO₂H, —CO₂-ethyl, acetoxymethyl, —Br, —Cl, -methylsulfanyl (—S—CH₃), nitrophenyl, chlorophenyl and -[1,3]-dioxolan. Particularly preferred substituents for “cycloalkyl” are CO₂H and CO₂-ethyl. Preferred substituents for “heterocyclyl” are methyl and ethyl.

Pharmaceutically acceptable salts within the scope of this invention are those salts of the compounds according to the invention having the general formula I which, when used pharmaceutically, are physiologically tolerable—especially when administered to mammals, especially humans. Such pharmaceutically acceptable salts can be formed, for example, with inorganic or organic acids.

The pharmaceutically acceptable salts of the compounds according to the invention having the general formula I are preferably formed with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid. The salts that are formed are, inter alia, hydrochlorides, hydrobromides, phosphates, carbonates, hydrogen carbonates, formates, acetates, oxalates, succinates, tartrates, fumarates, citrates and glutamates. Also preferred are solvates and, especially, the hydrates of the compounds according to the invention, which can be obtained, for example, by crystallization from aqueous solution.

If the compounds having the general formula I have at least one center of asymmetry, they may be in the form of their racemates, in the form of the a enantiomer and/or a pure diastereoisomer, or in the form of a mixture of enantiomers or diastereoisomers. The mixtures can be present in any desired mixing ratio of the stereoisomers. Chiral compounds having the general formula I are preferably enantiomerically pure compounds.

For the preparation according to the invention of a medicament for inhibiting NOS, for the treatment of migraine or for the treatment of septic shock, multiple sclerosis, Parkinson's disease, Alzheimer's disease, Huntington's disease, inflammations, inflammatory pain, cerebral ischemia, diabetes, meningitis, arteriosclerosis and/or for healing wounds, it is preferred to use those compounds having the general formula I (in the form of their bases or of their pharmaceutically acceptable salts) in which

R¹ represents methyl, ethyl, n-propyl, 2-propyl, n-butyl, tert-butyl, n-hexyl, n-octyl, 1,1,3,3-tetramethylbutyl; CH₂-aryl, wherein the aryl is unsubstituted or mono- or poly-substituted; CH₂CO₂—C₁₋₆-alkyl, wherein the alkyl is straight-chain or branched, CH₂PO(O—C₁₋₆-alkyl)₂; wherein the alkyl is straight-chain or branched; CH₂SiR¹²R¹³R¹⁴, CH₂CH₂-morpholin-4-yl, (CH₂)ₙ—NC, wherein n=2, 3, 4, 5 or 6, C₃₋₈-cycloalkyl, wherein the cycloalkyl is unsubstituted or mono- or poly-substituted, or phenyl, wherein the phenyl is unsubstituted or mono- or poly-substituted,

R² represents H or C(═O)—C 14-alkyl,

R³ represents methyl, ethyl, n-propyl, 2-propyl, n-butyl, tert-butyl, cyclopropyl, cyclopentyl or cyclohexyl, which are unsubstituted or mono- or poly-substituted; phenyl, which is unsubstituted or mono- or poly-substituted; 1-naphthyl or 2-naphthyl, wherein the naphthyl is unsubstituted or mono- or poly-substituted; 9-phenanthrenyl, pyrrol-2-yl, pyrrol-3-yl, pyridin-2-yl, pyridin-3-yl or pyridin-4-yl, wherein the pyrrolyl or pyridinyl are unsubstituted or mono- or poly-substituted; furan-2-yl or furan-3-yl, wherein the furanyl is unsubstituted or mono- or poly-substituted; thien-2-yl or thien-3-yl, wherein the thienyl is unsubstituted or mono- or poly-substituted; imidazol-2-yl, imidazol-4-yl, imidazol-5-yl, wherein the imidazolyl is unsubstituted or mono- or poly-substituted; thiazol-2-yl, thiazol-4-yl, thiazol-5-yl, wherein the thiazolyl is unsubstituted or mono- or poly-substituted; oxazol-2-yl, oxazol-4-yl, oxazol-5-yl, wherein the oxazolyl is unsubstituted or mono- or poly-substituted; isooxazol-3-yl, isooxazol-4-yl, isooxazol-5-yl, wherein the isooxazolyl is unsubstituted or mono- or poly-substituted; indol-2-yl, benzofuran-2-yl, or benzofuran-3-yl,

R⁴, R⁵, R⁶ and R⁷ each independently of the others represents H, methyl, ethyl, n-propyl, 2-propyl, n-butyl, tert-butyl, CF₃, F, Cl, Br, I, CO₂H, CO₂-methyl, CO₂ethyl, C(═O)CH₃ or NO₂, or R⁶ and R⁷ form the hydrocarbon bridge —CH═CH—CH═CH—,

R⁸ represents C(═O)CH₃ and

R¹², R¹³ and R¹⁴ each independently of the others represents C₁₋₆-alkyl, which is straight-chain or branched and is unsubstituted or mono- or poly-substituted; C₃₋₈-cycloalkyl or CH₂-C₃₋₈-cycloalkyl, wherein the cycloalkyl is unsubstituted or mono- or poly-substituted; or phenyl, which is unsubstituted or mono- or poly-substituted.

Further preference is given to the use according to the invention of compounds having the general formula I in which

R¹ represents methyl, ethyl, n-propyl, 2-propyl, n-butyl, tert-butyl, n-hexyl, n-octyl, 1,1,3,3-tetramethylbutyl, CH₂-aryl, wherein the aryl is unsubstituted or mono- or poly-substituted; CH₂CO₂—C₁₋₆-alkyl, wherein the alkyl is straight-chain or branched; CH₂PO(O—C₁₋₆-alkyl)₂, wherein the alkyl is straight-chain or branched; CH₂SiR¹²R¹³R¹⁴, CH₂CH₂-morpholin-4-yl, (CH₂)ₙ—NC, wherein n=2, 3, 4, 5 or 6; C₃₋₈-cycloalkyl, wherein the cycloalkyl is unsubstituted or mono- or poly-substituted; or phenyl, wherein the phenyl is unsubstituted or mono- or poly-substituted,

R² represents H or C(═O)—C₁₋₄-alkyl,

R³ represents methyl, ethyl, n-propyl, 2-propyl, n-butyl, tert-butyl, cyclopropyl, cyclopentyl or cyclohexyl, which, independently of one another, are unsubstituted or mono- or poly-substituted; phenyl, which is unsubstituted or monosubstituted or poly-substituted by identical or different substituents selected from methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy and dimethylamino; 1-naphthyl or 2-naphthyl, wherein naphthyl is unsubstituted or monosubstituted or poly-substituted by identical or different substituents selected from methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy and dimethylamino; 9-phenanthrenyl, pyrrol-2-yl, pyridin-2-yl, pyridin-3-yl or pyridin-4-yl, wherein pyridinyl is unsubstituted or monosubstituted or poly-substituted by identical or different substituents selected from methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; furan-2-yl or furan-3-yl, wherein furanyl is unsubstituted or monosubstituted or poly-substituted by identical or different substituents selected from methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; thien-2-yl or thien-3-yl, wherein thienyl is unsubstituted or monosubstituted or poly-substituted by identical or different substituents selected from methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; indol-2-yl, benzofuran-2-yl or benzofuran-3-yl,

R⁴, R⁵, R⁶ and R⁷ each independently of the others represents H, methyl, ethyl, n-propyl, 2-propyl, n-butyl, tert-butyl, CF₃, F, Cl, Br, I, CO₂H, CO₂-methyl, CO₂-ethyl, C(═O)CH₃ or NO₂, or R⁶ and R⁷ form the hydrocarbon bridge CH═CH—CH═CH—,

R⁸ represents C(═O)CH₃ and

R¹², R¹³ and R¹⁴ each independently of the others represents C₁₋₆-alkyl, which is straight-chain or branched and is unsubstituted or mono- or poly-substituted; C₃₋₈-cycloalkyl or CH₂-C₃₋₈-cycloalkyl, wherein the cycloalkyl is unsubstituted or mono- or poly-substituted; or phenyl, wherein the phenyl is unsubstituted or mono- or poly-substituted.

Particular preference is given to the use according to the invention of compounds having the general formula I in which

R¹ represents methyl, n-butyl, 1,1,3,3-tetramethylbutyl, benzyl, 2-chlorobenzyl, 2-methoxybenzyl, CH₂CO₂CH₃, (CH₂)₆—NC, cyclopentyl, cyclohexyl, phenyl, 2,6-dimethylphenyl, 3-chlorophenyl or 3-chloro-4-fluorophenyl,

R² represents H or C(═O)CH₃,

R³ represents methyl, tert-butyl, cyclohexyl, phenyl, 2-methylphenyl, 3-methylphenyl, 4-methylphenyl, 2-trifluorO-methylphenyl, 3-trifluorO-methylphenyl, 4-trifluorO-methylphenyl, 2-hydroxyphenyl, 2-methoxyphenyl, 3-hydroxyphenyl, 3-methoxyphenyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 2-nitrophenyl, 3-nitrophenyl, 4-nitrophenyl, 3-(4-chlorophenoxy)-phenyl, 2,4-dimethylphenyl, 2,3-dimethoxyphenyl, 3,4-dimethoxyphenyl, 3-methoxy-4-acetoxyphenyl, 2,3-dichlorophenyl, 2,4-dichlorophenyl, 2-chloro-4-fluorophenyl, 2-chloro-6-fluorophenyl, 4-bromo-2-fluorophenyl, 3,4,5-trimethoxyphenyl, 1-naphthyl, 2-ethoxy-naphth-1-yl, 4-dimethylamino-napth-1-yl, 9-phenanthrenyl, pyrrol-2-yl, N-methylpyrrol-2-yl, pyridin-2-yl, pyridin-3-yl, pyridin-4-yl, furan-2-yl, furan-3-yl, 5-methyl-furan-2-yl, 4,5-dimethyl-furan-2-yl, 5-hydroxymethyl-furan-2-yl, 5-acetoxymethyl-furan-2-yl, 5-carboxy-furan-2-yl, 5-[1,3]-dioxolan-furan-2-yl, 3-bromo-furan-2-yl, 5-bromo-furan-2-yl, 5-nitro-furan-2-yl, 5-(2-nitrophenyl)-furan-2-yl, 5-(2-chlorophenyl)-furan-2-yl, 5-(3-chlorophenyl)-furan-2-yl, 5-(3-chlorophenyl)-furan-3-yl, 5-(4-chlorophenyl)-furan-2-yl, benzo[b]furan-2-yl, thien-2-yl, thien-3-yl, 5-methyl-thien-2-yl, 5-carboxy-thien-2-yl, 3-bromo-thien-2-yl, 5-chloro-thien-2-yl or 5-methylsulfanyl-thien-2-yl,

R⁴ represents H, CH₃, Cl, Br or CO₂H,

R⁵ represents H, CH₃, C₂H₅ or Cl,

R⁶ represents H, CH₃, Cl, Br or NO₂,

R⁷ represents H, CH₃ or n-C₃H₇ and

R⁸ represents C(═O)CH₃.

Very particular preference is given to the use of compounds in which R⁴ and R⁶ represent H, R⁵ represents H, CH₃ or C₂H₅ and R⁷ represents H or CH₃.

The compounds having the general formula I (in the form of their bases or of their pharmaceutically acceptable salts), which are to be used for the preparation according to the invention of a medicament for inhibiting NOS, for the treatment of migraine or for the treatment of septic shock, multiple sclerosis, Parkinson's disease, Alzheimer's disease, Huntington's disease, inflammations, inflammatory pain, cerebral ischemia, diabetes, meningitis, arteriosclerosis and/or for healing wounds, are preferably selected from the group containing:

tert-butyl-(7-methyl-2-pyridin-3-yl-imidazo[1,2-a]pyridin-3-yl)-amine,

cyclohexyl-(5,7-dimethyl-2-pyridin-4-yl-imidazo[1,2-a]pyridin-3-yl)-amine,

(5,7-dimethyl-2-pyridin-4-yl-imidazo[1,2-a]pyridin-3-yl)-(1,1,3,3-tetramethylbutyl)-amine,

{6-[5,7-dimethyl-2-(1H-pyrrol-2-yl)-imidazo[1,2-a]pyridin-3-ylamino]-hexyl}-methylidyne-ammonium,

tert-butyl-[2-(2,3-dimethoxy-phenyl)-5,7-dimethyl-imidazo[1,2-a]pyridin-3-yl]-amine,

[2-(3,4-dimethoxy-phenyl)-7-methyl-imidazo[1,2-a]pyridin-3-yl]-(1,1,3,3-tetramethyl-butyl)-amine,

cyclohexyl-(7-methyl-2-pyridin-4-yl-imidazo[1,2-a]pyridin-3-yl)-amine,

(2-furan-2-yl-5,7-dimethyl-imidazo[1,2-a]pyridin-3-yl)-(1,1,3,3-tetramethylbutyl)-amine,

(1,1,3,3-tetramethyl-butyl)-(2,5,7-trimethyl-imidazo[1,2-a]pyridin-3-yl)-amine,

cyclohexyl-(7-methyl-2-o-tolyl-imidazo[1,2-a]pyridin-3-yl)-amine,

cyclohexyl-(7-methyl-2-thiophen-2-yl-imidazo[1,2-a]pyridin-3-yl)-amine,

(5,7-dimethyl-2-pyridin-3-yl-imidazo[1,2-a]pyridin-3-yl)-(1,1,3,3-tetramethylbutyl)-amine,

cyclohexyl-[7-methyl-2-(2-trifluorO-methyl-phenyl)-imidazo[1,2-a]pyridin-3-yl]-amine,

tert-butyl-(2,5,7-trimethyl-imidazo[1,2-a]pyridin-3-yl)-amine,

(7-methyl-2-pyridin-3-yl-imidazo[1,2-a]pyridin-3-yl)-(1,1,3,3-tetramethyl-butyl)-amine,

cyclohexyl-(7-methyl-2-naphthalen-1-yl-imidazo[1,2-a]pyridin-3-yl)-amine,

[2-(2-fluoro-phenyl)-7-methyl-imidazo[1,2-a]pyridin-3-yl]-(1,1,3,3-tetramethylbutyl)-amine,

(2,7-dimethyl-imidazo[1,2-a]pyridin-3-ylamino)-acetic acid methyl ester,

methylidyne-[6-(7-methyl-2-pyridin-3-yl-imidazo[1,2-a]pyridin-3-ylamino)-hexyl]-ammonium,

3-(3-tert-butylamino-5,7-dimethyl-imidazo[1,2-a]pyridin-2-yl)-phenol,

cyclohexyl-[2-(2-fluoro-phenyl)-7-methyl-imidazo[1,2-a]pyridin-3-yl]-amine,

tert-butyl-(2-cyclohexyl-5,7-dimethyl-imidazo[1,2-a]pyridin-3-yl)-amine,

cyclohexyl-(7-methyl-2-pyridin-3-yl-imidazo[1,2-a]pyridin-3-yl)-amine,

3-(3-tert-butylamino-7-methyl-imidazo[1,2-a]pyridin-2-yl)-phenol,

tert-butyl-(2-furan-2-yl-5,7-dimethyl-imidazo[1,2-a]pyridin-3-yl)-amine,

cyclohexyl-(2-furan-2-yl-5,7-dimethyl-imidazo[1,2-a]pyridin-3-yl)-amine,

cyclohexyl-(5,7-dimethyl-2-pyridin-3-yl-imidazo[1,2-a]pyridin-3-yl)-amine,

(2-furan-2-yl-7-methyl-imidazo[1,2-a]pyridin-3-yl)-(1,1,3,3-tetramethyl-butyl)-amine,

(7-methyl-2-pyridin-4-yl-imidazo[1,2-a]pyridin-3-yl)-(1,1,3,3-tetramethyl-butyl)-amine,

butyl-(2-cyclohexyl-7-methyl-imidazo[1,2-a]pyridin-3-yl)-amine,

3-[5,7-dimethyl-3-(1,1,3,3-tetramethyl-butylamino)-imidazo[1,2-a]pyridin-2-yl]-phenol,

(2,6-dimethyl-phenyl)-(5,7-dimethyl-2-o-tolyl-imidazo[1,2-a]pyridin-3-yl)-amine,

tert-butyl-(7-methyl-2-naphthalen-1-yl-imidazo[1,2-a]pyridin-3-yl)-amine,

(2,6-dimethyl-phenyl)-[2-(2-fluoro-phenyl)-5,7-dimethyl-imidazo[1,2-a]pyridin-3-yl]-amine,

cyclohexyl-(2,5,7-trimethyl-imidazo[1,2-a]pyridin-3-yl)-amine,

[5,7-dimethyl-2-(1H-pyrrol-2-yl)-imidazo[1,2-a]pyridin-3-yl]-(1,1,3,3-tetramethyl-butyl)-amine,

butyl-(2-cyclohexyl-5,7-dimethyl-imidazo[1,2-a]pyridin-3-yl)-amine,

(5,7-dimethyl-2-o-tolyl-imidazo[1,2-a]pyridin-3-yl)-(1,1,3,3-tetramethyl-butyl)-amine,

[2-(2,3-dimethoxy-phenyl)-7-methyl-imidazo[1,2-a]pyridin-3-yl]-(2,6-dimethylphenyl)-amine,

(2,7-dimethyl-imidazo[1,2-a]pyridin-3-yl)-(1,1,3,3-tetramethyl-butyl)-amine,

[2-(5-[1,3]dioxolan-2-yl-furan-2-yl)-7-methyl-imidazo[1,2-a]pyridin-3-yl]-(1,1,3,3-tetramethyl-butyl)-amine,

[2-(3-bromo-thiophen-2-yl)-5,7-dimethyl-imidazo[1,2-a]pyridin-3-yl]-(2,6-dimethyl-phenyl)-amine,

(2,6-dimethyl-phenyl)-[2-(2-fluoro-phenyl)-7-methyl-imidazo[1,2-a]pyridin-3-yl]-amine,

(2-cyclohexyl-5,7-dimethyl-imidazo[1,2-a]pyridin-3-yl)-(1,1,3,3-tetramethylbutyl)-amine,

[6-(2-furan-2-yl-5,7-dimethyl-imidazo[1,2-a]pyridin-3-ylamino)-hexyl]-methylidyne-ammonium,

(7-methyl-2-o-tolyl-imidazo[1,2-a]pyridin-3-yl)-(1,1,3,3-tetramethyl-butyl)-amine,

[2-(2,3-dichloro-phenyl)-7-methyl-imidazo[1,2-a]pyridin-3-yl]-(2,6-dimethylphenyl)-amine,

[2-(2,3-dimethoxy-phenyl)-5,7-dimethyl-imidazo[1,2-a]pyridin-3-yl]-(2,6-dimethyl-phenyl)-amine,

butyl-[2-(2,3-dimethoxy-phenyl)-5,7-dimethyl-imidazo[1,2-a]pyridin-3-yl]-amine,

methylidyne-[6-(7-methyl-2-p-tolyl-imidazo[1,2-a]pyridin-3-ylamino)-hexyl]-ammonium,

tert-butyl-[5,7-dimethyl-2-(5-nitro-furan-2-yl)-imidazo[1,2-a]pyridin-3-yl]-amine,

acetic acid 5-(3-cyclohexylamino-5,7-dimethyl-imidazo[1,2-a]pyridin-2-yl)-furan-2-yl methyl ester,

[2-(2-methoxy-phenyl)-5,7-dimethyl-imidazo[1,2-a]pyridin-3-yl]-(1,1,3,3-tetramethyl-butyl)-amine,

[2-(3,4-dimethoxy-phenyl)-7-methyl-imidazo[1,2-a]pyridin-3-yl]-(2,6-dimethylphenyl)-amine,

3-(3-butylamino-7-methyl-imidazo[1,2-a]pyridin-2-yl)-phenol,

(2-benzofuran-2-yl-7-methyl-imidazo[1,2-a]pyridin-3-yl)-(1,1,3,3-tetramethylbutyl)-amine,

(2-benzofuran-2-yl-5,7-dimethyl-imidazo[1,2-a]pyridin-3-yl)-(2,6-dimethylphenyl)-amine,

acetic acid 5-(3-cyclohexylamino-7-methyl-imidazo[1,2-a]pyridin-2-yl)-furan-2-yl methyl ester,

[6-(5,7-dimethyl-2-pyridin-3-yl-imidazo[1,2-a]pyridin-3-ylamino)-hexyl]-methylidyne-ammonium,

butyl-[2-(2-methoxy-phenyl)-7-methyl-imidazo[1,2-a]pyridin-3-yl]-amine,

{6-[2-(2-methoxy-phenyl)-5,7-dimethyl-imidazo[1,2-a]pyridin-3-ylamino]-hexyl}-methylidyne-ammonium,

{5-[5,7-dimethyl-3-(1,1,3,3-tetramethyl-butylamino)-imidazo[1,2-a]pyridin-2-yl]-furan-2-yl}-methanol,

(7-methyl-2-naphthalen-1-yl-imidazo[1,2-a]pyridin-3-yl)-(1,1,3,3-tetramethylbutyl)-amine,

[5-(3-tert-butylamino-7-methyl-imidazo[1,2-a]pyridin-2-yl)-furan-2-yl]-methanol,

tert-butyl-[2-(5-[1,3]dioxolan-2-yl-furan-2-yl)-7-methyl-imidazo[1,2-a]pyridin-3-yl]-amine,

(2-benzofuran-2-yl-5,7-dimethyl-imidazo[1,2-a]pyridin-3-yl)-(1,1,3,3-tetramethyl-butyl)-amine,

5-(3-tert-butylamino-5,7-dimethyl-imidazo[1,2-a]pyridin-2-yl)-furan-2-carboxylic acid,

tert-butyl-(2-furan-2-yl-7-methyl-imidazo[1,2-a]pyridin-3-yl)-amine,

cyclohexyl-(2,7-dimethyl-imidazo[1,2-a]pyridin-3-yl)-amine,

[2-(2,3-dichlorophenyl)-8-methyl-imidazo[1,2-a]pyridin-3-yl]-(1,1,3,3-tetramethyl-butyl)-amine,

(7-methyl-2-p-tolyl-imidazo[1,2-a]pyridin-3-yl)-(1,1,3,3-tetramethyl-butyl)-amine,

(2,6-dimethyl-phenyl)-[2-(2-methoxy-phenyl)-7-methyl-imidazo[1,2-a]pyridin-3-yl]-amine,

3-(3-butylamino-5,7-dimethyl-imidazo[1,2-a]pyridin-2-yl)-phenol,

butyl-[2-(2,3-dimethoxy-phenyl)-7-methyl-imidazo[1,2-a]pyridin-3-yl]-amine,

{6-[5,7-dimethyl-2-(2-trifluorO-methyl-phenyl)-imidazo[1,2-a]pyridin-3-ylamino]-hexyl}-methylidyne-ammonium,

tert-butyl-(7-methyl-2-pyridin-2-yl-imidazo[1,2-a]pyridin-3-yl)-amine,

cyclohexyl-(2-cyclohexyl-5,7-dimethyl-imidazo[1,2-a]pyridin-3-yl)-amine,

[2-(2,3-dichlorophenyl)-5,7-dimethyl-imidazo[1,2-a]pyridin-3-yl]-(2,6-dimethylphenyl)-amine,

(2,6-dimethyl-phenyl)-[2-(2-methoxy-phenyl)-5,7-dimethyl-imidazo[1,2-a]-pyridin-3-yl]-amine,

{2-[5-(2-chlorophenyl)-furan-2-yl]-7-methyl-imidazo[1,2-a]pyridin-3-yl}-(1,1,3,3-tetramethyl-butyl)-amine,

5-[7-methyl-3-(1,1,3,3-tetramethyl-butylamino)-imidazo[1,2-a]pyridin-2-yl]-furan-2-carboxylic acid,

cyclohexyl-[2-(2-methoxy-phenyl)-8-methyl-imidazo[1,2-a]pyridin-3-yl]-amine,

3-[7-methyl-3-(1,1,3,3-tetramethyl-butylamino)-imidazo[1,2-a]pyridin-2-yl]-phenol,

[2-(2,3-dichlorophenyl)-5,7-dimethyl-imidazo[1,2-a]pyridin-3-yl]-(1,1,3,3-tetramethyl-butyl)-amine,

[2-(2,4-dichlorophenyl)-7-methyl-imidazo[1,2-a]pyridin-3-yl]-(1,1,3,3-tetramethyl-butyl)-amine,

[2-(5-bromofuran-2-yl)-7-methyl-imidazo[1,2-a]pyridin-3-yl]-(1,1,3,3-tetramethyl-butyl)-amine,

5-(3-cyclohexylamino-5,7-dimethyl-imidazo[1,2-a]pyridin-2-yl)-furan-2-carboxylic acid,

[6-(2-cyclohexyl-5,7-dimethyl-imidazo[1,2-a]pyridin-3-ylamino)-hexyl]-methylidyne-ammonium,

[2-(2,4-dichlorophenyl)-5,7-dimethyl-imidazo[1,2-a]pyridin-3-yl]-(1,1,3,3-tetramethyl-butyl)-amine,

(2-benzofuran-2-yl-7-methyl-imidazo[1,2-a]pyridin-3-yl)-(2,6-dimethyl-phenyl)-amine,

5-(3-cyclohexylamino-7-methyl-imidazo[1,2-a]pyridin-2-yl)-furan-2-carboxylic acid,

{6-[2-(2-bromophenyl)-5,7-dimethyl-imidazo[1,2-a]pyridin-3-ylamino]-hexyl}-methylidyne-ammonium,

tert-butyl-(2-cyclohexyl-7-methyl-imidazo[1,2-a]pyridin-3-yl)-amine,

tert-butyl-(2,7-dimethyl-imidazo[1,2-a]pyridin-3-yl)-amine,

(5,7-dimethyl-2-p-tolyl-imidazo[1,2-a]pyridin-3-yl)-(1,1,3,3-tetramethyl-butyl)-amine,

[2-(2,3-dichlorophenyl)-8-methyl-imidazo[1,2-a]pyridin-3-yl]-(2,6-dimethylphenyl)-amine,

methylidyne-[6-(7-methyl-2-o-tolyl-imidazo[1,2-a]pyridin-3-ylamino)-hexyl]-ammonium,

{2-[5-(3-chlorophenyl)-furan-2-yl]-7-methyl-imidazo[1,2-a]pyridin-3-yl}-(1,1,3,3-tetramethyl-butyl)-amine,

cyclohexyl-[7-methyl-2-(5-nitro-furan-2-yl)-imidazo[1,2-a]pyridin-3-yl]-amine,

[2-(2-bromophenyl)-7-methyl-imidazo[1,2-a]pyridin-3-yl]-cyclohexyl-amine,

[2-(2-methoxy-phenyl)-7-methyl-imidazo[1,2-a]pyridin-3-yl]-(1,1,3,3-tetramethyl-butyl)-amine,

{5-[7-methyl-3-(1,1,3,3-tetramethyl-butylamino)-imidazo[1,2-a]pyridin-2-yl]-furan-2-yl}-methanol,

(6-{2-[5-(2-chlorophenyl)-furan-2-yl]-5-methyl-imidazo[1,2-a]pyridin-3-ylamino}-hexyl)-methylidyne-ammonium,

cyclohexyl-[5,7-dimethyl-2-(5-nitro-furan-2-yl)-imidazo[1,2-a]pyridin-3-yl]-amine,

cyclohexyl-[2-(4,5-dimethyl-furan-2-yl)-7-methyl-imidazo[1,2-a]pyridin-3-yl]-amine,

[6-(5,7-dimethyl-2-o-tolyl-imidazo[1,2-a]pyridin-3-ylamino)-hexyl]-methylidyne-ammonium,

methylidyne-[6-(7-methyl-2-pyridin-2-yl-imidazo[1,2-a]pyridin-3-ylamino)-hexyl]-ammonium,

[2-(2,3-dimethoxy-phenyl)-5,7-dimethyl-imidazo[1,2-a]pyridin-3-yl]-(1,1,3,3-tetramethyl-butyl)-amine,

{6-[2-(3,4-dimethoxy-phenyl)-7-methyl-imidazo[1,2-a]pyridin-3-ylamino]-hexyl}-methylidyne-ammonium,

5-(3-tert-butylamino-7-methyl-imidazo[1,2-a]pyridin-2-yl)-thiophene-2-carboxylic acid,

cyclohexyl-(8-methyl-2-pyridin-4-yl-imidazo[1,2-a]pyridin-3-yl)-amine,

[2-(2,3-dichloro-phenyl)-6-methyl-imidazo[1,2-a]pyridin-3-yl]-(1,1,3,3-tetramethyl-butyl)-amine,

5-(3-butylamino-imidazo[1,2-a]pyrazin-2-yl)-thiophene-2-carboxylic acid,

cyclohexyl-(5,7-dimethyl-2-pyridin-2-yl-imidazo[1,2-a]pyridin-3-yl)-amine,

(2-benzofuran-2-yl-8-methyl-imidazo[1,2-a]pyridin-3-yl)-(1,1,3,3-tetramethylbutyl)-amine,

{6-[2-(2-fluoro-phenyl)-5,7-dimethyl-imidazo[1,2-a]pyridin-3-ylamino]-hexyl}-methylidyne-ammonium,

[2-(2,3-dimethoxy-phenyl)-8-methyl-imidazo[1,2-a]pyridin-3-yl]-(1,1,3,3-tetramethyl-butyl)-amine,

methylidyne-[6-(7-methyl-2-phenanthren-9-yl-imidazo[1,2-a]pyridin-3-ylamino)-hexyl]-ammonium,

5-(3-tert-butylamino-imidazo[1,2-a]pyrazin-2-yl)-thiophene-2-carboxylic acid,

tert-butyl-(8-methyl-2-pyridin-2-yl-imidazo[1,2-a]pyridin-3-yl)-amine,

cyclohexyl-(2-furan-2-yl-8-methyl-imidazo[1,2-a]pyridin-3-yl)-amine,

methylidyne-[6-(7-methyl-2-naphthalen-1-yl-imidazo[1,2-a]pyridin-3-ylamino)-hexyl]-ammonium,

tert-butyl-(2-cyclohexyl-8-methyl-imidazo[1,2-a]pyridin-3-yl)-amine,

(6-methyl-2-pyridin-4-yl-imidazo[1,2-a]pyridin-3-yl)-(1,1,3,3-tetramethyl-butyl)-amine,

tert-butyl-(6-methyl-2-pyridin-3-yl-imidazo[1,2-a]pyridin-3-yl)-amine,

(7-methyl-2-pyridin-3-yl-imidazo[1,2-a]pyrimidin-3-yl)-(1,1,3,3-tetramethylbutyl)-amine,

5-(3-tert-butylamino-6-methyl-imidazo[1,2-a]pyridin-2-yl)-thiophene-2-carboxylic acid,

[6-(5,7-dimethyl-2-naphthalen-1-yl-imidazo[1,2-a]pyridin-3-ylamino)-hexyl]-methylidyne-ammonium,

3-[3-(2,6-dimethyl-phenylamino)-5,7-dimethyl-imidazo[1,2-a]pyridin-2-yl]-phenol,

(2,6-dimethyl-phenyl)-(8-methyl-2-o-tolyl-imidazo[1,2-a]pyridin-3-yl)-amine,

{6-[2-(3-hydroxy-phenyl)-8-methyl-imidazo[1,2-a]pyridin-3-ylamino]-hexyl}-methylidyne-ammonium,

{5-[3-(2,6-dimethyl-phenylamino)-7-methyl-imidazo[1,2-a]pyrimidin-2-yl]-furan-2-yl}-methanol,

(8-methyl-2-p-tolyl-imidazo[1,2-a]pyridin-3-yl)-(1,1,3,3-tetramethyl-butyl)-amine,

[2-(2,4-dichlorophenyl)-6-methyl-imidazo[1,2-a]pyridin-3-yl]-(2,6-dimethylphenyl)-amine,

butyl-[2-(2,4-dichloro-phenyl)-6-methyl-imidazo[1,2-a]pyridin-3-yl]-amine,

butyl-[2-(4-dimethylamino-naphthalen-1-yl)-imidazo[1,2-a]pyrazin-3-yl]-amine,

{6-[2-(2-bromo-phenyl)-7-methyl-imidazo[1,2-a]pyridin-3-ylamino]-hexyl}-methylidyne-ammonium,

butyl-[2-(2-methoxy-phenyl)-6-methyl-imidazo[1,2-a]pyridin-3-yl]-amine,

(2-cyclohexyl-8-methyl-imidazo[1,2-a]pyridin-3-yl)-(1,1,3,3-tetramethyl-butyl)-amine,

cyclohexyl-(7-methyl-2-pyridin-2-yl-imidazo[1,2-a]pyridin-3-yl)-amine,

cyclohexyl-(2-furan-2-yl-7-methyl-imidazo[1,2-a]pyridin-3-yl)-amine,

(2-cyclohexyl-5,7-dimethyl-imidazo[1,2-a]pyridin-3-ylamino)-acetic acid methyl ester,

N-(2-furan-2-yl-5,7-dimethyl-imidazo[1,2-a]pyridin-3-yl)-N-(1,1,3,3-tetramethylbutyl)-acetamide,

N-tert-butyl-N-(7-methyl-2-pyridin-3-yl-imidazo[1,2-a]pyridin-3-yl)-acetamide,

N-tert-butyl-N-(2-furan-2-yl-5,7-dimethyl-imidazo[1,2-a]pyridin-3-yl)-acetamide,

N-(5,7-dimethyl-2-pyridin-4-yl-imidazo[1,2-a]pyridin-3-yl)-N-(1,1,3,3-tetramethyl-butyl)-acetamide,

N-(5,7-dimethyl-2-pyridin-3-yl-imidazo[1,2-a]pyridin-3-yl)-N-(1,1,3,3-tetramethyl-butyl)-acetamide,

N-(2,6-dimethyl-phenyl)-N-(5,7-dimethyl-2-o-tolyl-imidazo[1,2-a]pyridin-3-yl)-acetamide,

N-(2-furan-2-yl-7-methyl-imidazo[1,2-a]pyridin-3-yl)-N-(1,1,3,3-tetramethylbutyl)-acetamide,

N-(1,1,3,3-tetramethyl-butyl)-N-(2,5,7-trimethyl-imidazo[1,2-a]pyridin-3-yl)-acetamide,

N-cyclohexyl-N-(7-methyl-2-thiophen-2-yl-imidazo[1,2-a]pyridin-3-yl)-acetamide,

N-tert-butyl-N-(2,5,7-trimethyl-imidazo[1,2-a]pyridin-3-yl)-acetamide,

5-[3-(acetyl-tert-butyl-amino)-imidazo[1,2-a]pyrazin-2-yl]-thiophene-2-carboxylic acid,

5-{3-[acetyl-(1,1,3,3-tetramethyl-butyl)-amino]-5,7-dimethyl-imidazo[1,2-a]-pyridin-2-yl}-furan-2-carboxylic acid,

N-[2-(5-hydroxymethyl-furan-2-yl)-5,7-dimethyl-imidazo[1,2-a]pyridin-3-yl]-N-(1,1,3,3-tetramethyl-butyl)-acetamide,

N-[2-(3-bromo-thiophen-2-yl)-5,7-dimethyl-imidazo[1,2-a]pyridin-3-yl]-N-(2,6-dimethyl-phenyl)-acetamide,

N-tert-butyl-N-(2-cyclohexyl-5,7-dimethyl-imidazo[1,2-a]pyridin-3-yl)-acetamide,

acetic acid 5-[3-(acetyl-cyclohexyl-amino)-5,7-dimethyl-imidazo[1,2-a]pyridin-2-yl]-furan-2-yl methyl ester,

{6-[acetyl-(2-furan-2-yl-5,7-dimethyl-imidazo[1,2-a]pyridin-3-yl)-amino]-hexyl}-methylidyne-ammonium,

N-[2-(2,3-dichloro-phenyl)-7-methyl-imidazo[1,2-a]pyridin-3-yl]-N-(2,6-dimethyl-phenyl)-acetamide,

N-[2-(3-bromo-thiophen-2-yl)-imidazo[1,2-a]pyridin-3-yl]-N-cyclohexyl-acetamide,

N-(5,7-dimethyl-2-p-tolyl-imidazo[1,2-a]pyridin-3-yl)-N-(1,1,3,3-tetramethylbutyl)-acetamide,

N-cyclohexyl-N-(7-methyl-2-pyridin-4-yl-imidazo[1,2-a]pyridin-3-yl)-acetamide,

N-cyclohexyl-N-[7-methyl-2-(2-trifluorO-methyl-phenyl)-imidazo[1,2-a]pyridin-3-yl]-acetamide,

N-(6,8-dibromo-2-furan-2-yl-imidazo[1,2-a]pyridin-3-yl)-N-(1,1,3,3-tetramethylbutyl)-acetamide,

N-(7-methyl-2-pyridin-4-yl-imidazo[1,2-a]pyridin-3-yl)-N-(1,1,3,3-tetramethylbutyl)-acetamide,

acetic acid 5-[3-(acetyl-cyclohexyl-amino)-7-methyl-imidazo[1,2-a]pyridin-2-yl]-furan-2-yl methyl ester,

N-(7-methyl-2-pyridin-3-yl-imidazo[1,2-a]pyridin-3-yl)-N-(1,1,3,3-tetramethylbutyl)-acetamide,

N-[2-(2,3-dichloro-phenyl)-5,7-dimethyl-imidazo[1,2-a]pyridin-3-yl]-N-(2,6-dimethyl-phenyl)-acetamide,

N-cyclohexyl-N-[5,7-dimethyl-2-(5-methyl-furan-2-yl)-imidazo[1,2-a]pyridin-3-yl]-acetamide,

N-butyl-N-[2-(2,3-dimethoxy-phenyl)-5,7-dimethyl-imidazo[1,2-a]pyridin-3-yl]-acetamide,

N-[2-(2-methoxy-phenyl)-5,7-dimethyl-imidazo[1,2-a]pyridin-3-yl]-N-(1,1,3,3-tetramethyl-butyl)-acetamide,

N-cyclohexyl-N-[5,7-dimethyl-2-(5-nitro-furan-2-yl)-imidazo[1,2-a]pyridin-3-yl]-acetamide,

[acetyl-(2,7-dimethyl-imidazo[1,2-a]pyridin-3-yl)-amino]-acetic acid methyl ester,

N-cyclohexyl-N-(2,5,7-trimethyl-imidazo[1,2-a]pyridin-3-yl)-acetamide,

5-{3-[acetyl-(1,1,3,3-tetramethyl-butyl)-amino]-imidazo[1,2-a]pyridin-2-yl}-thiophene-2-carboxylic acid,

N-[2-(2,4-dichloro-phenyl)-5,7-dimethyl-imidazo[1,2-a]pyridin-3-yl]-N-(1,1,3,3-tetramethyl-butyl)-acetamide,

N-cyclohexyl-N-[7-methyl-2-(5-nitro-furan-2-yl)-imidazo[1,2-a]pyridin-3-yl]-acetamide,

N-(2-tert-butyl-5,7-dimethyl-imidazo[1,2-a]pyridin-3-yl)-N-(2,6-dimethylphenyl)-acetamide,

N-(2,6-dimethyl-phenyl)-N-[2-(2-methoxy-phenyl)-5,7-dimethyl-imidazo[1,2-a]-pyridin-3-yl]-acetamide,

N-[2-(3-hydroxy-phenyl)-5,7-dimethyl-imidazo[1,2-a]pyridin-3-yl]-N-(1,1,3,3-tetramethyl-butyl)-acetamide,

N-(2,6-dimethyl-phenyl)-N-[2-(2-fluoro-phenyl)-5,7-dimethyl-imidazo[1,2-a]-pyridin-3-yl]-acetamide,

5-[3-(acetyl-tert-butyl-amino)-5-methyl-imidazo[1,2-a]pyridin-2-yl]-thiophene-2-carboxylic acid,

N-(2,6-dimethyl-phenyl)-N-[2-(2-methoxy-phenyl)-7-methyl-imidazo[1,2-a]-pyridin-3-yl]-acetamide,

N-(7-methyl-2-p-tolyl-imidazo[1,2-a]pyridin-3-yl)-N-(1,1,3,3-tetramethyl-butyl)-acetamide,

5-{3-[acetyl-(1,1,3,3-tetramethyl-butyl)-amino]-7-methyl-imidazo[1,2-a]pyridin-2-yl}-furan-2-carboxylic acid,

N-cyclohexyl-N-(7-methyl-2-pyridin-3-yl-imidazo[1,2-a]pyridin-3-yl)-acetamide,

N-[2-(5-[1,3]dioxolan-2-yl-furan-2-yl)-5,7-dimethyl-imidazo[1,2-a]pyridin-3-yl]-N-(1,1,3,3-tetramethyl-butyl)-acetamide,

N-(2-benzofuran-2-yl-5,7-dimethyl-imidazo[1,2-a]pyridin-3-yl)-N-(2,6-dimethylphenyl)-acetamide,

N-tert-butyl-N-(2-furan-2-yl-7-methyl-imidazo[1,2-a]pyridin-3-yl)-acetamide,

N-tert-butyl-N-(7-methyl-2-pyridin-2-yl-imidazo[1,2-a]pyridin-3-yl)-acetamide,

N-cyclohexyl-N-(5,7-dimethyl-2-pyridin-3-yl-imidazo[1,2-a]pyridin-3-yl)-acetamide,

N-[2-(2,3-dichloro-phenyl)-5,7-dimethyl-imidazo[1,2-a]pyridin-3-yl]-N-(1,1,3,3-tetramethyl-butyl)-acetamide,

N-[2-(2,3-dimethoxy-phenyl)-5,7-dimethyl-imidazo[1,2-a]pyridin-3-yl]-N-(1,1,3,3-tetramethyl-butyl)-acetamide,

N-{2-[3-(4-chloro-phenoxy)-phenyl]-imidazo[1,2-a]pyridin-3-yl}-N-(2,6-dimethyl-phenyl)-acetamide,

N-[2-(5-[1,3]dioxolan-2-yl-furan-2-yl)-7-methyl-imidazo[1,2-a]pyridin-3-yl]-N-(1,1,3,3-tetramethyl-butyl)-acetamide,

5-[3-(acetyl-cyclohexyl-amino)-5,7-dimethyl-imidazo[1,2-a]pyridin-2-yl]-furan-2-carboxylic acid,

N-tert-butyl-N-[7-methyl-2-(5-nitro-furan-2-yl)-imidazo[1,2-a]pyridin-3-yl]-acetamide,

N-[2-(2-methoxy-phenyl)-7-methyl-imidazo[1,2-a]pyridin-3-yl]-N-(1,1,3,3-tetramethyl-butyl)-acetamide,

N-[2-(5-methyl-furan-2-yl)-imidazo[1,2-a]pyridin-3-yl]-N-(1,1,3,3-tetramethylbutyl)-acetamide,

5-[3-(acetyl-tert-butyl-amino)-7-methyl-imidazo[1,2-a]pyridin-2-yl]-furan-2-carboxylic acid,

N-[2-(4,5-dimethyl-furan-2-yl)-imidazo[1,2-a]pyridin-3-yl]-N-(1,1,3,3-tetramethyl-butyl)-acetamide,

N-cyclohexyl-N-(2-furan-2-yl-5,7-dimethyl-imidazo[1,2-a]pyridin-3-yl)-acetamide,

N-tert-butyl-N-(7-methyl-2-naphthalen-1-yl-imidazo[1,2-a]pyridin-3-yl)-acetamide,

5-{3-[acetyl-(1,1,3,3-tetramethyl-butyl)-amino]-imidazo[1,2-a]pyrazin-2-yl}-thiophene-2-carboxylic acid,

N-butyl-N-(2-o-tolyl-imidazo[1,2-a]pyrimidin-3-yl)acetamide,

N-[2-(3,4-dimethoxy-phenyl)-7-methyl-imidazo[1,2-a]pyridin-3-yl]-N-(2,6-dimethyl-phenyl)-acetamide,

N-tert-butyl-N-(7-methyl-2-phenanthren-9-yl-imidazo[1,2-a]pyridin-3-yl)-acetamide,

N-(2,6-dimethyl-phenyl)-N-[2-(2-fluoro-phenyl)-7-methyl-imidazo[1,2-a]pyridin-3-yl]-acetamide,

N-[2-(2-methoxy-phenyl)-8-methyl-imidazo[1,2-a]pyridin-3-yl]-N-(1,1,3,3-tetramethyl-butyl)-acetamide,

N-(2,6-dimethyl-phenyl)-N-[2-(3-hydroxy-phenyl)-5,7-dimethyl-imidazo[1,2-a]-pyridin-3-yl]-acetamide,

N-(2-tert-butyl-7-methyl-imidazo[1,2-a]pyridin-3-yl)-N-(2,6-dimethyl-phenyl)-acetamide,

acetic acid 4-{3-[acetyl-(2,6-dimethyl-phenyl)-amino]-6-bromo-8-methyl-imidazo[1,2-a]pyridin-2-yl}-2-methoxy-phenyl ester,

N-tert-butyl-N-[2-(5-[1,3]dioxolan-2-yl-furan-2-yl)-7-methyl-imidazo[1,2-a]-pyridin-3-yl]-acetamide,

[6-(acetyl-{7-methyl-2-[5-(2-nitro-phenyl)-furan-2-yl]-imidazo[1,2-a]pyridin-3-yl}-amino)-hexyl]-methylidyne-ammonium,

N-(2-benzofuran-2-yl-7-methyl-imidazo[1,2-a]pyridin-3-yl)-N-(1,1,3,3-tetramethyl-butyl)-acetamide,

N-(2-benzofuran-2-yl-5,7-dimethyl-imidazo[1,2-a]pyridin-3-yl)-N-(1,1,3,3-tetramethyl-butyl)-acetamide,

5-[3-(acetyl-tert-butyl-amino)-imidazo[1,2-a]pyridin-2-yl]-thiophene-2-carboxylic acid,

N-(2-cyclohexyl-5,7-dimethyl-imidazo[1,2-a]pyridin-3-yl)-N-(1,1,3,3-tetramethyl-butyl)-acetamide,

N-tert-butyl-N-[2-(5-methyl-furan-2-yl)-imidazo[1,2-a]pyridin-3-yl]-acetamide,

N-tert-butyl-N-[2-(5-methylsulfanyl-thiophen-2-yl)-imidazo[1,2-a]pyrimidin-3-yl]-acetamide,

N-[2-(4,5-dimethyl-furan-2-yl)-5-methyl-imidazo[1,2-a]pyridin-3-yl]-N-(1,1,3,3-tetramethyl-butyl)-acetamide,

N-butyl-N-[2-(2,4-dichloro-phenyl)-imidazo[1,2-a]pyrimidin-3-yl]-acetamide,

N-[2-(3-bromo-thiophen-2-yl)-6-methyl-imidazo[1,2-a]pyridin-3-yl]-N-cyclohexyl-acetamide,

5-{3-[acetyl-(2,6-dimethyl-phenyl)-amino]-6-methyl-imidazo[1,2-a]pyridin-2-yl}-thiophene-2-carboxylic acid,

N-butyl-N-[2-(2,3-dimethoxy-phenyl)-7-methyl-imidazo[1,2-a]pyridin-3-yl]-acetamide,

N-tert-butyl-N-[2-(2,3-dichloro-phenyl)-imidazo[1,2-a]pyridin-3-yl]-acetamide,

N-(2-furan-2-yl-5-propyl-imidazo[1,2-a]pyridin-3-yl)-N-(1,1,3,3-tetramethylbutyl)-acetamide,

5-[3-(acetyl-cyclohexyl-amino)-imidazo[1,2-a]pyridin-2-yl]-thiophene-2-carboxylic acid,

5-{3-[acetyl-(1,1,3,3-tetramethyl-butyl)-amino]-8-methyl-imidazo[1,2-a]pyridin-2-yl}-furan-2-carboxylic acid,

3-(acetyl-butyl-amino)-2-pyridin-2-yl-imidazo[1,2-a]pyridine-8-carboxylic acid,

{6-[acetyl-(5,7-dimethyl-2-pyridin-3-yl-imidazo[1,2-a]pyridin-3-yl)-amino]-hexyl}-methylidyne-ammonium,

N-tert-butyl-N-[2-(5-methylsulfanyl-thiophen-2-yl)-imidazo[1,2-a]pyrazin-3-yl]-acetamide,

5-[3-(acetyl-cyclohexyl-amino)-5-methyl-imidazo[1,2-a]pyridin-2-yl]-thiophene-2-carboxylic acid,

N-[2-(5-methylsulfanyl-thiophen-2-yl)-imidazo[1,2-a]pyrazin-3-yl]-N-(1,1,3,3-tetramethyl-butyl)-acetamide,

N-[2-(2,3-dichloro-phenyl)-5-methyl-imidazo[1,2-a]pyridin-3-yl]-N-(2,6-dimethyl-phenyl)-acetamide,

N-butyl-N-[2-(2-methoxy-phenyl)-imidazo[1,2-a]pyridin-3-yl]-acetamide,

(6-{acetyl-[2-(2-methoxy-phenyl)-6-nitro-imidazo[1,2-a]pyridin-3-yl]-amino}-hexyl)-methylidyne-ammonium,

N-(2-benzofuran-2-yl-7-methyl-imidazo[1,2-a]pyridin-3-yl)-N-(2,6-dimethylphenyl)-acetamide,

(6-{acetyl-[2-(2-methoxy-phenyl)-5,7-dimethyl-imidazo[1,2-a]pyridin-3-yl]-amino}-hexyl)-methylidyne-ammonium,

{6-[acetyl-(7-methyl-2-pyridin-3-yl-imidazo[1,2-a]pyridin-3-yl)-amino]-hexyl}-methylidyne-ammonium,

N-(6-methyl-2-p-tolyl-imidazo[1,2-a]pyridin-3-yl)-N-(1,1,3,3-tetramethyl-butyl)-acetamide,

acetic acid 5-{3-[acetyl-(2,6-dimethyl-phenyl)-amino]-5,7-dimethyl-imidazo[1,2-a]pyridin-2-y}-furan-2-yl methyl ester,

{acetyl-[2-(3-hydroxy-phenyl)-6-methyl-imidazo[1,2-a]pyridin-3-yl]-amino}-acetic acid methyl ester,

N-tert-butyl-N-[2-(2-trifluorO-methyl-phenyl)-imidazo[1,2-a]pyridin-3-yl]-acetamide,

N-butyl-N-[2-(2-chloro-4-fluoro-phenyl)-imidazo[1,2-a]pyridin-3-yl]-acetamide,

N-[2-(2,4-dichloro-phenyl)-imidazo[1,2-a]pyridin-3-yl]-N-(2,6-dimethyl-phenyl)-acetamide,

5-{3-[acetyl-(1,1,3,3-tetramethyl-butyl)-amino]-7-methyl-imidazo[1,2-a]-pyrimidin-2-yl}-furan-2-carboxylic acid,

acetic acid 5-{3-[acetyl-(1,1,3,3-tetramethyl-butyl)-amino]-imidazo[1,2-a]-pyrimidin-2-yl}-furan-2-yl methyl ester,

N-(2,7-dimethyl-imidazo[1,2-a]pyridin-3-yl)-N-(1,1,3,3-tetramethyl-butyl)-acetamide,

acetic acid 4-[3-(acetyl-cyclohexyl-amino)-5-amino-7-chloro-imidazo[1,2-a]-pyrimidin-2-yl]-2-methoxy-phenyl ester,

acetic acid 4-[3-(acetyl-cyclohexyl-amino)-5,7-dimethyl-imidazo[1,2-a]-pyrimidin-2-yl]-2-methoxy-phenyl ester,

N-[6-bromo-2-(2-chloro-6-fluoro-phenyl)-8-methyl-imidazo[1,2-a]pyridin-3-yl]-N-cyclohexyl-acetamide,

N-[2-(2-chloro-6-fluoro-phenyl)-5-methyl-imidazo[1,2-a]pyridin-3-yl]-N-cyclohexyl-acetamide,

N-butyl-N-[2-(2,3-dichloro-phenyl)-imidazo[1,2-a]pyrimidin-3-yl]-acetamide,

N-[2-(5-chloro-thiophen-2-yl)-imidazo[1,2-a]pyrimidin-3-yl]-N-(1,1,3,3-tetramethyl-butyl)-acetamide,

[acetyl-(2-cyclohexyl-7-methyl-imidazo[1,2-a]pyridin-3-yl)-amino]-acetic acid methyl ester,

N-tert-butyl-N-[2-(2-chloro-6-fluoro-phenyl)-imidazo[1,2-a]pyridin-3-yl]-acetamide,

N-cyclohexyl-N-(5,7-dimethyl-2-pyridin-2-yl-imidazo[1,2-a]pyridin-3-yl)-acetamide,

acetic acid 5-[3-(acetyl-cyclohexyl-amino)-5-methyl-imidazo[1,2-a]pyridin-2-yl]-furan-2-yl methyl ester,

N-(2,6-dimethyl-phenyl)-N-[6-methyl-2-(2-trifluorO-methyl-phenyl)-imidazo[1,2-a]pyridin-3-yl]-acetamide,

N-cyclohexyl-N-(2-furan-2-yl-7-methyl-imidazo[1,2-a]pyridin-3-yl)-acetamide,

N-cyclohexyl-N-(7-methyl-2-pyridin-2-yl-imidazo[1,2-a]pyridin-3-yl)-acetamide,

N-cyclohexyl-N-[2-(5-[1,3]dioxolan-2-yl-furan-2-yl)-5-methyl-imidazo[1,2-a]-pyridin-3-yl]-acetamide,

N-tert-butyl-N-(5-propyl-2-pyridin-3-yl-imidazo[1,2-a]pyridin-3-yl)-acetamide,

N-tert-butyl-N-[2-(5-methyl-thiophen-2-yl)-imidazo[1,2-a]pyrimidin-3-yl]-acetamide,

3-[acetyl-(1,1,3,3-tetramethyl-butyl)-amino]-2-furan-2-yl-imidazo[1,2-a]pyridine-8-carboxylic acid,

N-tert-butyl-N-[2-(4,5-dimethyl-furan-2-yl)-6-methyl-imidazo[1,2-a]pyridin-3-yl]-acetamide,

N-{2-[3-(4-chloro-phenoxy)-phenyl]-imidazo[1,2-a]pyridin-3-yl}-N-cyclohexyl-acetamide,

acetic acid 4-[3-(acetyl-cyclohexyl-amino)-imidazo[1,2-a]pyrimidin-2-yl]-2-methoxy-phenyl ester,

N-[2-(5-bromo-furan-2-yl)-8-methyl-imidazo[1,2-a]pyridin-3-yl]-N-(1,1,3,3-tetramethyl-butyl)-acetamide,

N-(2,6-dimethyl-phenyl)-N-[2-(3-hydroxy-phenyl)-5,7-dimethyl-imidazo[1,2-a]-pyrimidin-3-yl]-acetamide,

N-cyclohexyl-N-[2-(2,3-dichloro-phenyl)-imidazo[1,2-a]pyridin-3-yl]-acetamide,

N-cyclohexyl-N-[2-(2,4-dichloro-phenyl)-5-methyl-imidazo[1,2-a]pyridin-3-yl]-acetamide,

N-cyclohexyl-N-[2-(2,4-dichloro-phenyl)-imidazo[1,2-a]pyrimidin-3-yl]-acetamide,

[acetyl-(2-o-tolyl-imidazo[1,2-a]pyrazin-3-yl)-amino]-acetic acid methyl ester,

N-tert-butyl-N-(6,8-dichloro-2-thiophen-2-yl-imidazo[1,2-a]pyridin-3-yl)-acetamide,

N-tert-butyl-N-(5-propyl-2-thiophen-2-yl-imidazo[1,2-a]pyridin-3-yl)-acetamide,

{6-[acetyl-(7-methyl-2-p-tolyl-imidazo[1,2-a]pyridin-3-yl)-amino]-hexyl}-methylidyne-ammonium,

N-butyl-N-(6-methyl-2-p-tolyl-imidazo[1,2-a]pyridin-3-yl)-acetamide,

(6-{acetyl-[2-(2-methoxy-phenyl)-7-methyl-imidazo[1,2-a]pyridin-3-yl]-amino}-hexyl)-methylidyne-ammonium,

5-{3-[acetyl-(1,1,3,3-tetramethyl-butyl)-amino]-6-methyl-imidazo[1,2-a]pyridin-2-yl}-furan-2-carboxylic acid,

N-butyl-N-[2-(3,4,5-trimethoxy-phenyl)-imidazo[1,2-a]pyrazin-3-yl]-acetamide,

N-butyl-N-[2-(3-hydroxy-phenyl)-imidazo[1,2-a]pyrimidin-3-yl]-acetamide,

[acetyl-(2-o-tolyl-imidazo[1,2-a]pyrimidin-3-yl)-amino]-acetic acid methyl ester,

N-(2-benzofuran-2-yl-8-methyl-imidazo[1,2-a]pyridin-3-yl)-N-(1,1,3,3-tetramethyl-butyl)-acetamide,

N-butyl-N-(7-methyl-2-p-tolyl-imidazo[1,2-a]pyrimidin-3-yl)-acetamide,

N-tert-butyl-N-(6,8-dibromo-2-methyl-imidazo[1,2-a]pyridin-3-yl)-acetamide,

{6-[acetyl-(5,7-dimethyl-2-pyridin-2-yl-imidazo[1,2-a]pyridin-3-yl)-amino]-hexyl}-methylidyne-ammonium,

N-tert-butyl-N-[2-(2-ethoxy-naphthalen-1-yl)-7-methyl-imidazo[1,2-a]pyridin-3-yl]-acetamide,

N-tert-butyl-N-[2-(2-chloro-4-fluoro-phenyl)-imidazo[1,2-a]pyridin-3-yl]-acetamide,

cyclohexyl-[7-methyl-2-(2-trifluorO-methyl-phenyl)-imidazo[1,2-a]pyridin-3-yl]-amine hydrochloride,

tert-butyl-(2-furan-2-yl-5,7-dimethyl-imidazo[1,2-a]pyridin-3-yl)-amine hydrochloride,

tert-butyl-(7-methyl-2-phenyl-imidazo[1,2-a]pyridin-3-yl)-amine hydrochloride,

cyclohexyl-(5,7-dimethyl-2-pyridin-4-yl-imidazo[1,2-a]pyridin-3-yl)-amine hydrochloride,

(2-furan-2-yl-5,7-dimethyl-imidazo[1,2-a]pyridin-3-yl)-(1,1,3,3-tetramethylbutyl)-amine hydrochloride,

tert-butyl-(2,5,7-trimethyl-imidazo[1,2-a]pyridin-3-yl)-amine hydrochloride,

[2-(2-fluorophenyl)-7-methyl-imidazo[1,2-a]pyridin-3-yl]-(1,1,3,3-tetramethylbutyl)-amine hydrochloride,

cyclohexyl-(7-methyl-2-phenyl-imidazo[1,2-a]pyridin-3-yl)-amine hydrochloride,

(2-furan-2-yl-7-methyl-imidazo[1,2-a]pyrimidin-3-yl)-(1,1,3,3-tetramethylbutyl)-amine hydrochloride,

tert-butyl-[2-(4-nitro-phenyl)-imidazo[1,2-a]pyrazin-3-yl]-amine hydrochloride,

N-{2-[3-(4-chlorophenoxy)-phenyl]-imidazo[1,2-a]pyridin-3-yl}-N-cyclohexyl-acetamide hydrochloride,

N-cyclohexyl-N-(7-methyl-2-o-tolyl-imidazo[1,2-a]pyridin-3-yl)-acetamide hydrochloride,

N-(2,6-dimethyl-phenyl)-N-[2-(2,4-dimethyl-phenyl)-5-methyl-imidazo[1,2-a]-pyridin-3-yl]-acetamide hydrochloride,

1-acetyl-3-(acetyl-cyclohexyl-amino)-7-methyl-2-o-tolyl-imidazo[1,2-a]pyridin-1-ium) chloride hydrochloride,

cyclohexyl-(2-furan-3-yl-5,7-dimethyl-imidazo[1,2-a]pyridin-3-yl)-amine hydrochloride,

cyclopentyl-(2-furan-3-yl-5,7-dimethyl-imidazo[1,2-a]pyridin-3-yl)-amine hydrochloride,

[2-(4-bromo-2-fluoro-phenyl)-5,7-dimethyl-imidazo[1,2-a]pyridin-3-yl]-cyclopentyl-amine hydrochloride,

cyclopentyl-{5,7-dimethyl-2-[5-(2-nitro-phenyl)-furan-2-yl]-imidazo[1,2-a]-pyridin-3-yl}-amine hydrochloride,

{2-[5-(4-chlorophenyl)-furan-2-yl]-5,7-dimethyl-imidazo[1,2-a]pyridin-3-yl}-cyclopentyl-amine hydrochloride,

cyclopentyl-(2-furan-3-yl-5,7-dimethyl-imidazo[1,2-a]pyridin-3-yl)-amine hydrochloride,

(2-furan-3-yl-5,7-dimethyl-imidazo[1,2-a]pyridin-3-yl)-(1,1,3,3-tetramethylbutyl)-amine hydrochloride,

benzyl-(7-methyl-2-thiophen-3-yl-imidazo[1,2-a]pyridin-3-yl)-amine hydrochloride,

cyclohexyl-(2-furan-3-yl-5,7-dimethyl-imidazo[1,2-a]pyridin-3-yl)-amine hydrochloride,

(2-furan-3-yl-7-methyl-imidazo[1,2-a]pyridin-3-yl)-(1,1,3,3-tetramethyl-butyl)-amine hydrochloride,

(5,7-dimethyl-2-thiophen-3-yl-imidazo[1,2-a]pyridin-3-yl)-(1,1,3,3-tetramethylbutyl)-amine hydrochloride,

[7-ethyl-2-(5-nitro-furan-2-yl)-imidazo[1,2-a]pyridin-3-yl]-(2-methoxybenzyl)-amine,

(2-chlorobenzyl)-[7-ethyl-2-(5-nitro-furan-2-yl)-imidazo[1,2-a]pyridin-3-yl]-amine,

[7-ethyl-2-(5-methyl-furan-2-yl)-imidazo[1,2-a]pyridin-3-yl]-(2-methoxybenzyl)-amine,

(2-chlorobenzyl)-(7-ethyl-2-furan-2-yl-imidazo[1,2-a]pyridin-3-yl)-amine,

(3-chloro-4-fluorophenyl)-[7-ethyl-2-(5-methyl-furan-2-yl)-imidazo[1,2-a]-pyridin-3-yl]-amine,

(2-benzofuran-2-yl-7-ethyl-imidazo[1,2-a]pyridin-3-yl)-(3-chloro-4-fluorophenyl)-amine,

(2-benzofuran-2-yl-7-ethyl-imidazo[1,2-a]pyridin-3-yl)-(3-chlorophenyl)-amine,

(3-chloro-4-fluorophenyl)-{2-[5-(3-chloro-phenyl)-furan-2-yl]-7-ethyl-imidazo[1,2-a]pyridin-3-yl}-amine,

(3-chloro-4-fluorophenyl)-{2-[5-(2-chlorophenyl)-furan-2-yl]-7-ethyl-imidazo[1,2-a]pyridin-3-yl}-amine, and

(3-chloro-4-fluorophenyl)-[2-(4,5-dimethyl-furan-2-yl)-7-ethyl-imidazo[1,2-a]-pyridin-3-yl]-amine.

The compounds used according to the invention having the general formula I

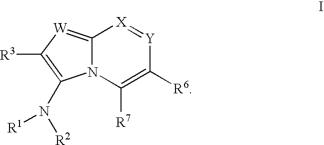

in which R² represents hydrogen, W represents N and R¹, R³, R⁶, R⁷, X and Y have the meanings given above, i.e. compounds having the general formula Ia, can be prepared according to the following reaction equation:

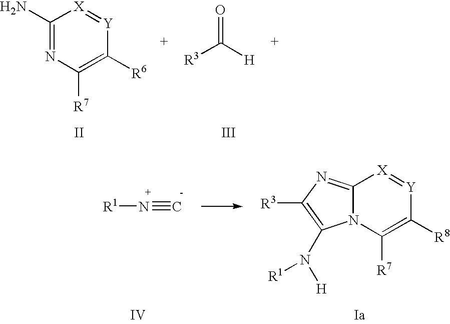

In that reaction, an amidine having the general formula II, i.e. an aminopyridine (X=CR⁴ and Y=CR⁵) or an aminopyrimidine (X=N and Y=CR⁵) or an aminopyrazine (X=CR⁴ and Y=N), wherein the radicals R⁴ to R⁷ are as defined for the compound having the general formula I, is reacted under suitable reaction conditions with an aldehyde having the general formula III, wherein R³ is as defined for the compound having the general formula I, and with an isonitrile of the general formula IV, wherein R¹ is as defined for the compound having the general formula I.

The reaction is preferably carried out in the presence of a small amount of an acid, especially 20% aqueous perchloric acid, in a three-component one-pot reaction, which may also be carried out in parallel synthesis semi-automatically or fully automatically. The reaction is preferably carried out in an organic solvent, especially dichloromethane or acetonitrile, at a temperature of preferably from 0° C. to 80° C., especially from 15° C. to 30° C.

The starting compounds having the general structures II, III and IV are commercially available (e.g. from Acros, Geel; Avocado, Port of Heysham; Aldrich, Deisenhofen; Fluka, Seelze; Lancaster, Mülheim; Maybridge, Tintagel; Merck, Darmstadt; Sigma, Deisenhofen; TCI, Japan) and/or are readily obtainable according to processes well-known to those ordinarily skilled in the art.

For the preparation of the compounds used according to the invention of the general formula I in which R² does not represent hydrogen but represents C(═O)R⁹, the compounds having the general formula in which R² represents H (i.e. compounds of the general formula Ia) can be reacted, according to the desired end product, with an acid halide R⁹CO-Hal wherein Hal represents fluorine, chlorine, bromine or iodine and R⁹ is as defined above for the compound having the general formula I, either without a solvent or in a polar or non-polar aprotic solvent, for example dimethyl sulfoxide (DMSO), dimethylformamide (DMF), halogenated hydrocarbons, such as dichloromethane and acetonitrile, aliphatic ethers, such as tetrahydrofuran (THF) or 1,4-dioxan, or in hydrocarbons or in mixtures of those solvents, within a period of, for example, from about 0.25 to 12 hours at temperatures of from about 0° C. to 160° C., in accordance with the following reaction scheme:

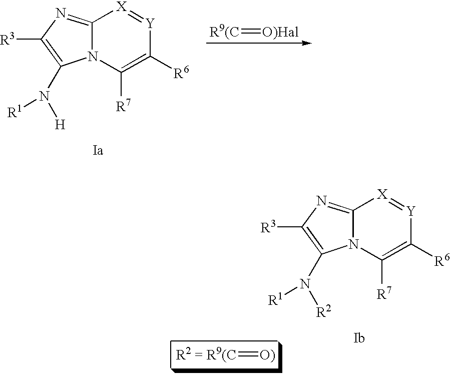

Alternatively, the compounds of the general formula Ia can be deprotonated at the exocyclic amino nitrogen by means of a strong base, for example an organometallic compound, such as n-butyllithium, in an aprotic solvent, such as DMF or DMSO, preferably in an ether, such as tetrahydrofuran or 1,4-dioxan, at temperatures of preferably from about −70° C. to +20° C. The subsequent addition of an acid halide yields the compounds of the general formula Ib, in which R² represents R⁹(C═O):

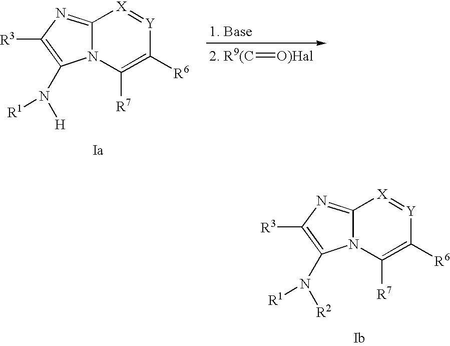

Reacting compounds having the general formula Ib with an acid halide again yields the compounds having the general formula I in which W represents NR⁸, i.e. compounds having the general formula Ic:

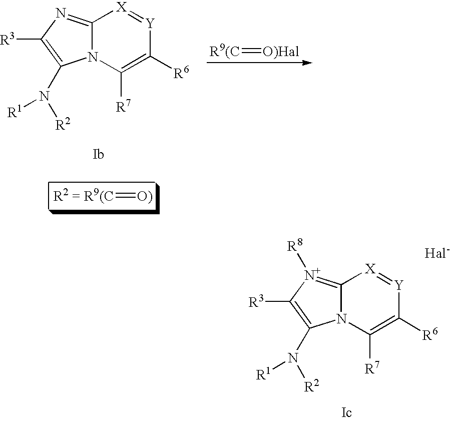

The compounds used according to the invention having the general formula I can be isolated either in the form of the free base or in the form of a salt. The free base of the compound having the general formula I used according to the invention is usually obtained after reaction according to the above-described process and subsequent conventional working-up. The free base so obtained or formed in situ without isolation can then be converted into the corresponding salt, for example by reaction with an inorganic or organic acid, preferably with hydrochloric acid, hydrobromic acid, sulfuric acid, phosphoric acid, methanesulfonic acid, p-toluenesulfonic acid, carbonic acid, formic acid, acetic acid, oxalic acid, succinic acid, tartaric acid, mandelic acid, fumaric acid, lactic acid, citric acid, glutamic acid or aspartic acid. The salts formed are, inter alia, hydrochlorides, hydrobromides, phosphates, carbonates, hydrogen carbonates, formates, acetates, oxalates, succinates, tartrates, fumarates, citrates and glutamates. Formation of the hydrochloride, which is particularly preferred, can also be effected by adding trimethylsilyl chloride (TMSCI) to the base dissolved in a suitable organic solvent, such as butan-2-one (methyl ethyl ketone).

If the compounds having the general formula I are obtained in the form of racemates or in the form of mixtures of their different enantiomers and/or diastereoisomers, such mixtures can be separated by processes which are well known in the art. Suitable methods are, inter alia, chromatographic separation processes, especially liquid chromatography processes under normal or elevated pressure, preferably MPLC and HPLC processes, and also fractional crystallization processes. By means of such processes it is possible especially to separate from one another, for example by means of HPLC on chiral phase or by means of crystallization, individual enantiomers of diastereoisomeric salts formed with chiral acids, for example (+)-tartaric acid, (−)-tartaric acid or (+)-10-camphorsulfonic acid.

The medicaments which can be prepared by the use according to the invention of the compounds having the general formula I and which are to be used for inhibiting NOS, for the treatment of migraine or for the treatment of septic shock, multiple sclerosis, Parkinson's disease, Alzheimer's disease, Huntington's disease, inflammations, inflammatory pain, cerebral ischemia, diabetes, meningitis, arteriosclerosis and/or for healing wounds, are usually pharmaceutical compositions which contain one or more pharmaceutical excipients in addition to at least one compound having the general formula I in the form of its base or of one of its pharmaceutically acceptable salts.

The pharmaceutical compositions can be in liquid, semi-solid or solid pharmaceutical dosage forms and can be administered in the form of, for example, injectable solutions, drops, juices, syrups, sprays, suspensions, granules, tablets, pellets, patches, capsules, plasters, suppositories, ointments, creams, lotions, gels, emulsions or aerosols, and, in addition to at least one compound having the general formula I, they contain, depending on the particular galenical form, pharmaceutical excipients, such as carriers, fillers, solvents, diluents, surface-active substances, colourings, preservatives, disintegrators, glidants, lubricants, flavourings and/or binders. Such excipients may be, for example: water, ethanol, 2-propanol, glycerol, ethylene glycol, propylene glycol, polyethylene glycol, polypropylene glycol, glucose, fructose, lactose, saccharose, dextrose, molasses, starch, modified starch, gelatin, sorbitol, inositol, mannitol, microcrystalline cellulose, methyl cellulose, carboxymethyl cellulose, cellulose acetate, shellac, cetyl alcohol, polyvinylpyrrolidone, paraffins, waxes, pharmaceutically acceptable natural and synthetic gums, acacia gum, alginates, dextran, saturated and unsaturated fatty acids, stearic acid, magnesium stearate, zinc stearate, glyceryl stearate, sodium lauryl sulfate, edible oils, sesame oil, coconut oil, groundnut oil, soybean oil, lecithin, sodium lactate, polyoxyethylene and polyoxypropylene fatty acid esters, sorbitan fatty acid esters, sorbic acid, benzoic acid, citric acid, ascorbic acid, tannic acid, sodium chloride, potassium chloride, magnesium chloride, calcium chloride, magnesium oxide, zinc oxide, silicon dioxide, titanium oxide, titanium dioxide, magnesium sulfate, zinc sulfate, calcium sulfate, potassium carbonate, calcium phosphate, dicalcium phosphate, potassium bromide, potassium iodide, talcum, kaolin, pectin, crospovidone, agar and bentonite.

The choice of excipients and the amounts thereof to be used depend on whether the medicament is to be administered orally, subcutaneously, parenterally, intravenously, intraperitoneally, intradermally, intramuscularly, intranasally, buccally, rectally or locally, for example to infections of the skin, the mucosa and of the eyes. For oral administration there are suitable, inter alia, preparations in the form of tablets, dragees, capsules, granules, drops, juices and syrups, and for parenteral and topical administration and for administration by inhalation there are suitable solutions, suspensions, readily reconstitutable dry preparations and also sprays. Compounds having the general formula I in a depot formulation in dissolved form or in a plaster, optionally with the addition of agents promoting penetration of the skin, are suitable preparations for percutaneous administration. Forms of preparation for oral or percutaneous administration may release the compounds having the general formula I in a delayed manner.

The medicaments and pharmaceutical compositions containing a compound having the general formula I are prepared by means, devices, methods and processes which are well known in the art of pharmaceutical formulation, as are described, for example, in “Remington's Pharmaceutical Sciences”, ed. A. R. Gennaro, 17th ed., Mack Publishing Company, Easton, Pa. (1985), especially in Part 8, Chapter 76 to 93.

Accordingly, for a solid formulation, for example, such as a tablet, the active ingredient of the medicament, i.e. a compound having the general formula I or a pharmaceutically acceptable salt thereof, can be mixed with a pharmaceutical carrier, for example conventional tablet constituents such as maize starch, lactose, saccharose, sorbitol, talcum, magnesium stearate, dicalcium phosphate or gum, and pharmaceutical diluents, such as water, in order to form a solid preformulation composition which contains a compound according to the invention or a pharmaceutically acceptable salt thereof in homogeneous distribution. Homogeneous distribution is here understood to mean that the active ingredient is distributed evenly throughout the entire preformulation composition, so that the latter can readily be divided into unit dose forms, such as tablets, pills or capsules, which each have the same effectiveness. The solid preformulation composition is then divided into unit dose forms. It is also possible for the tablets or pills of the medicament according to the invention or of the compositions according to the invention to be coated or otherwise compounded, in order to prepare a delayed-release dosage form. Suitable coating agents are, inter alia, polymeric acids and mixtures of polymeric acids with materials such as shellac, cetyl alcohol and/or cellulose acetate.

The amount of active ingredient to be administered to a patient varies and is dependent on the weight, the age and the history of the patient, and also on the mode of administration, the indication and the severity of the disease. Normally, from 0.1 to 5000 mg/kg, especially from 1 to 500 mg/kg, preferably from 2 to 250 mg/kg body weight of at least one compound having the general formula I are administered.

The assays used to determine the NOS inhibition effected by the compounds having the general formula I are described hereinbelow:

NOS Assay

General

This assay enables the percentage inhibition of NO synthase by an active ingredient to be determined by measuring the NOS activity under the action of the active ingredient. NO synthase is mixed together with radioactively labelled arginine and the active ingredient under suitable conditions. After terminating the NO-forming reaction at a given time, the amount of unconverted arginine is determined directly or indirectly. A comparison of that amount with the amount of arginine that remains in a mixture of NOS and arginine without the addition of active ingredient and under otherwise identical conditions gives the % inhibition of NO synthase by the tested active ingredient. This assay can be carried out as follows:

(a) incubation of NO synthase with labelled arginine as substrate in a reaction vessel,

(b) separation of the labelled arginine from the labelled citrulline optionally formed as a product of the enzymatic reaction, at a time at which the citrulline concentration is increasing,

(c) measurement of the amount of arginine remaining in each case.

Separation is carried out over a filter plate membrane.

This NOS assay is suitable especially for “high throughput screening” (HTS) on microtitre plates (MTP).

HTS-NOS Assay: General Procedure

In this HTS-NOS assay, radioactive arginine is used as substrate. The assay volume can be chosen in the range from 25 μl to 250 μl, according to the type of microtitre plate (MTP). Depending on the source of the enzyme used, co-factors and co-enzymes are added. Incubation of the batches on this microtitre plate (assay MTP) according to step (a) is carried out at room temperature and is for 5 to 60 minutes according to the enzyme activity used (units). At the end of the incubation (step (a)), the plate is placed into a cell harvester which is equipped with a MTP having a cation-exchanger membrane as the filter base (filter MTP). All batches of the assay MTP are transferred to the filter MTP and filtered off with suction over a cation-exchanger filter plate, a paper filter charged with phosphate groups. The filter MTP is then washed with buffer or water. By means of this procedure, the arginine substrate that remains is bound to the cation exchanger, while the enzymatically formed radioactive citrulline is washed out quantitatively. After drying the filter MTP and adding scintillation liquid, the bound arginine can be counted using a scintillation counter. An uninhibited NOS reaction is reflected in low radioactivity. An inhibited enzyme reaction means that the radioactive arginine has not been converted. This means that a high level of radioactivity is found on the filter.

Materials Used

arginine, L-[2,3,4-³H]-monohydrochloride; order no. NET-1123, NEN

anhydrous CaCl₂; order no. 2388.1000; Merck KGaA

1,4-dithiothreitol (DTT), order no. 708984; ROCHE

Na₂EDTA dihydrate; order no. 03680; FLUKA

HEPES, order no. H-3375; SIGMA

NADPH, tetrasodium salt; order no. 1585363; ROCHE

TRIS; order no. 93349; FLUKA

| | |
|---|---|
| Enzyme preparation buffer: | 50 mM tris-HCl with 1 mM EDTA: The |
| | pH value of the buffer was adjusted to 7.4 |
| | at 4° C. |
| Incubation buffer (medium): | 50 mM HEPES with 1 mM EDTA; 1.25 |
| | mM CaCl₂ and 1 mM dithiothreitol. |
| | The pH value of the buffer was adjusted to |
| | 7.4 at 25° C. |
| Washing medium: | H₂O |

Enzyme Preparation

Rat cerebella were used as the starting tissue. The animals were anaesthetized and sacrificed, the brain tissue, the cerebellum, was removed by dissection, 1 ml of enzyme preparation buffer was added per rat cerebellum (4° C.), and disintegration was carried out using a Polytron homogeniser for 1 minute at 6000 rpm, followed by centrifugation for 15 minutes at 4° C. and 20,000 g; the supernatant was then removed by decantation and frozen in portions at −80° C. (the precipitate was discarded).

Incubation Batch:

96-well MTP having a well capacity of <250 μl were used.

Pipetting sequence: see Table 1.

TABLE 1

| | Molarity | | |
|---|---|---|---|
| Substance | i.b. | μl | *Protein i.b. |
| Incubat. | — | 100 | — |
| buffer | | | |
| Test | variable; | variable; preferably | — |
| substance | preferably | 20 μl | |
| | 10⁻⁵ M | | |
| NADPH | 0.5 mM | 20 | — |
| Enzyme | — | variable; maximum | variable; |
| (see Ex. 3) | | volume of the enzyme | maximum usable protein |
| | | solution = 50 μl | amount = 100 μg |
| [³H] | variable; | variable; preferably | — |
| substrate | preferably | 10 μl | |
| | 50 nM | | |

| | |
|---|---|
| Final volume: | max. 250 μl |

*Protein determination according to O. H. Lowry et al.; J. Biol. Chem 193 265 (1951)
i.b. = in the batch

When the pipetting operation was complete, a cover was placed on the MTP (assay MTP), and incubation was at 25° C. (room temperature (RT)) for from 5 to 60 minutes, according to the amount and activity of the enzyme used.

The contents of the assay MTP were then transferred with the aid of a 96-well cell harvester to a 96-well cation exchanger MTP (filter MTP) and filtered off with suction. This was followed by washing once with 200 ml of H₂O (from a bath).

The plate was then dried for 1 hour at 60° C. in a drying cabinet. The underside of the filter MTP was then accurately sealed from beneath with a “back seal”. 35 μl of scintillation fluid per well were then added by means of a pipette. The upper side of the plate was also sealed with a “top seal”. After waiting for 1 hour, the plate was counted using a β counter.

In the case of HTS operation, the incubation medium, the NADPH and the enzyme solution were combined before the start of the pipetting step, in order to avoid the necessity of carrying out three separate pipetting operations, which is costly in terms of time.

The results obtained for exemplary compounds in the NOS assay are shown in Table 3.

Citrulline Assay

This assay was carried out as described by D. S. Bredt and S. H. Snyder (Proc. Natl. Acad. Sci. USA (1990), 87, 682-685). The results obtained for example compounds in the citrulline assay are shown in Table 4.

The following Examples serve to illustrate the invention further, without limiting it thereto.

### EXAMPLES

The compounds having the general formula I were prepared according to the following general synthesis procedures (GWP):

General Working Procedure 1 (GWP 1)

A round-bottomed glass test tube (diameter 16 mm, length 125 mm) having a thread was provided with a stirrer and closed by means of a screw lid having a septum. The test tube was placed on a reactor block adjusted to a temperature of 15° C. The following reagents were added in succession by means of a pipette:

1) 1 ml of a 0.1 M amidine II solution+10 μl of 20% aqueous HClO₄, in dichloromethane

2) 0.5 ml of a 0.3 M solution of the aldehyde III in dichloromethane

3) 0.575 ml of a 0.2 M isonitrile IV solution in dichloromethane.

The reaction mixture was stirred for 12 hours at 15° C. The reaction solution was then filtered off. The test tube was rinsed twice using 1 ml of dichloromethane and 200 μl of water each time.

3 ml of a 10% NaCl solution and 1.5 ml of dichloromethane were added to the reaction mixture, and the whole was mixed thoroughly. The organic phase was separated off, and the aqueous phase was extracted again using 1.5 ml of dichloromethane. The combined organic phases were dried over 2.4 g of MgSO₄ (granulated). The solvent was removed in a vacuum centrifuge.

The chemicals and solvents used were obtained commercially. Each substance was analyzed by ESI-MS and/or NMR.

Examples 1 to 142 and 313 to 322 prepared according to GWP 1 were tested in an automated manner in the HTS-NOS assay. The results are shown in Table 2.

TABLE 2

| Example | | HTS-NOS assay: % | Weight | Weight |
|---|---|---|---|---|
| No. | Compound | inhibition (10 μM) | calc. | found |
| 1 | Tert-butyl-(7-methyl-2-pyridin-3-yl- | 63 | 280.37 | 281.3 |
| | imidazo[1,2-a]pyridin-3-yl)-amine | | | |
| 2 | Cyclohexyl-(5,7-dimethyl-2-pyridin-4-yl- | 62 | 320.43 | 321.3 |
| | imidazo[1,2-a]pyridin-3-yl)-amine | | | |
| 3 | (5,7-Dimethyl-2-pyridin-4-yl-imidazo[1,2-a]- | 60 | 350.5 | 351.4 |
| | pyridin-3-yl)-(1,1,3,3-tetramethyl-butyl)- | | | |
| | amine | | | |
| 4 | {6-[5,7-Dimethyl-2-(1H-pyrrol-2-yl)- | 54 | 336.46 | 336.4 |
| | imidazo[1,2-a]pyridin-3-ylamino]-hexyl}- | | | |
| | methylidyne-ammonium | | | |
| 5 | Tert-butyl-[2-(2,3-dimethoxy-phenyl)-5,7- | 61 | 353.46 | 354.2 |
| | dimethyl-imidazo[1,2-a]pyridin-3-yl]-amine | | | |
| 6 | [2-(3,4-Dimethoxy-phenyl)-7-methyl- | 53 | 395.54 | 396.3 |
| | imidazo[1,2-a]pyridin-3-yl]-(1,1,3,3- | | | |
| | tetramethyl-butyl)-amine | | | |
| 7 | Cyclohexyl-(7-methyl-2-pyridin-4-yl- | 65 | 306.41 | 307.4 |
| | imidazo[1,2-a]pyridin-3-yl)-amine | | | |
| 8 | (2-Furan-2-yl-5,7-dimethyl-imidazo[1,2-a]- | 52 | 339.48 | 340.4 |
| | pyridin-3-yl)-(1,1,3,3-tetramethyl-butyl)- | | | |
| | amine | | | |
| 9 | (1,1,3,3-Tetramethyl-butyl)-(2,5,7-trimethyl- | 59 | 287.45 | 288.4 |
| | imidazol[1,2-a]pyridin-3-yl)-amine | | | |
| 10 | Cyclohexyl-(7-methyl-2-o-tolyl-imidazol[1,2- | 54 | 319.45 | 320.4 |
| | a]pyridin-3-yl)-amine | | | |
| 11 | Cyclohexyl-(7-methyl-2-thiophen-2-yl- | 60 | 311.45 | 312.4 |
| | imidazo[1,2-a]pyridin-3-yl)-amine | | | |
| 12 | (5,7-Dimethyl-2-pyridin-3-yl-imidazo[1,2-a]- | 61 | 350.5 | 351.3 |
| | pyridin-3-yl)-(1,1,3,3-tetramethyl-butyl)- | | | |
| | amine | | | |
| 13 | Cyclohexyl-[7-methyl-2-(2-trifluorO-methyl- | 57 | 373.42 | 374.5 |
| | phenyl)-imidazo[1,2-a]pyridin-3-yl]-amine | | | |
| 14 | Tert-butyl-(2,5,7-trimethyl-imidazo[1,2-a]- | 67 | 231.34 | 232.2 |
| | pyridin-3-yl)-amine | | | |
| 15 | (7-Methyl-2-pyridin-3-yl-imidazo[1,2-a]- | 59 | 336.48 | 337.4 |
| | pyridin-3-yl)-(1,1,3,3-tetramethyl-butyl)- | | | |
| | amine | | | |
| 16 | Cyclohexyl-(7-methyl-2-naphthalen-1-yl- | 56 | 355.48 | 356.6 |
| | imidazo[1,2-a]pyridin-3-yl)-amine | | | |
| 17 | [2-(2-Fluoro-phenyl)-7-methyl-imidazo[1,2- | 51 | 353.48 | 354.3 |
| | a]pyridin-3-yl]-(1,1,3,3-tetramethyl-butyl)- | | | |
| | amine | | | |
| 18 | (2,7-Dimethyl-imidazo[1,2-a]pyridin-3- | 50 | 233.27 | 234.3 |
| | ylamino)-acetic acid methyl ester | | | |
| 19 | Methylidyne-[6-(7-methyl-2-pyridin-3-yl- | 53 | 334.44 | 334.4 |
| | imidazo[1,2-a]pyridin-3-ylamino)-hexyl]- | | | |
| | ammonium | | | |
| 20 | 3-(3-Tert-butylamino-5,7-dimethyl- | 64 | 309.41 | 310.3 |
| | imidazo[1,2-a]pyridin-2-yl)-phenol | | | |
| 21 | Cyclohexyl-(2-(2-fluoro-phenyl)-7-methyl- | 53 | 323.41 | 324.5 |
| | imidazo[1,2-a]pyridin-3-yl]-amine | | | |
| 22 | Tert-butyl-(2-cyclohexyl-5,7-dimethyl- | 68 | 299.46 | 300.3 |
| | imidazo[1,2-a]pyridin-3-yl)-amine | | | |
| 23 | Cyclohexyl-(7-methyl-2-pyridin-3-yl- | 74 | 306.41 | 307.4 |
| | imidazo[1,2-a]pyridin-3-yl)-amine | | | |
| 24 | 3-(3-Tert-butylamino-7-methyl-imidazo[1,2- | 59 | 295.38 | 296.3 |
| | a]pyridin-2-yl)-phenol | | | |
| 25 | Tert-butyl-(2-furan-2-yl-5,7-dimethyl- | 60 | 283.37 | 284.2 |
| | imidazo[1,2-a]pyridin-3-yl)-amine | | | |
| 26 | Cyclohexyl-(2-furan-2-yl-5,7-dimethyl- | 62 | 309.41 | 310.4 |
| | imidazo[1,2-a]pyridin-3-yl)-amine | | | |
| 27 | Cyclohexyl-(5,7-dimethyl-2-pyridin-3-yl- | 62 | 320.43 | 321.3 |
| | imidazo[1,2-a]pyridin-3-yl)-amine | | | |
| 28 | (2-Furan-2-yl-7-methyl-imidazo[1,2-a]- | 50 | 325.45 | 326.3 |
| | pyridin-3-yl)-(1,1,3,3-tetramethyl-butyl)- | | | |
| | amine | | | |
| 29 | (7-Methyl-2-pyridin-4-yl-imidazo[1,2-a]- | 69 | 336.48 | 337.4 |
| | pyridin-3-yl)-(1,1,3,3-tetramethyl-butyl)- | | | |
| | amine | | | |
| 30 | Butyl-(2-cyclohexyl-7-methyl-imidazo[1,2- | 56 | 285.43 | 286.4 |
| | a]pyridin-3-yl)-amine | | | |
| 31 | 3-[5,7-Dimethyl-3-(1,1,3,3-tetramethyl- | 60 | 365.52 | 366.3 |
| | butylamino)-imidazo[1,2-a]pyridin-2-yl]- | | | |
| | phenol | | | |
| 32 | (2,6-Dimethyl-phenyl)-(5,7-dimethyl-2-o- | 59 | 355.48 | 356.3 |
| | tolyl-imidazo[1,2-a]pyridin-3-yl)-amine | | | |
| 33 | Tert-butyl-(7-methyl-2-naphthalen-1-yl- | 67 | 329.44 | 330.4 |
| | imidazo[1,2-a]pyridin-3-yl)-amine | | | |
| 34 | (2,6-Dimethyl-phenyl)-[2-(2-fluoro-phenyl)- | 61 | 359.44 | 360.3 |
| | (5,7-dimethyl-imidazo[1,2-a]pyridin-3-yl)- | | | |
| | amine | | | |
| 35 | Cyclohexyl-(2,5,7-trimethyl-imidazo[1,2-a]- | 55 | 257.38 | 258.4 |
| | pyridin-3-yl)-amine | | | |
| 36 | [5,7-Dimethyl-2-(1H-pyrrol-2-yl)-imidazo- | 69 | 338.49 | 339.5 |
| | [1,2-a]pyridin-3-yl]-(1,1,3,3-tetramethyl- | | | |
| | butyl)-amine | | | |
| 37 | Butyl-(2-cyclohexyl-5,7-dimethyl- | 58 | 299.46 | 300.3 |
| | imidazo[1,2-a]pyridin-3-yl)-amine | | | |
| 38 | (5,7-Dimethyl-2-o-tolyl-imidazo[1,2-a]- | 60 | 363.54 | 364.3 |
| | pyridin-3-yl)-(1,1,3,3-tetramethyl-butyl)- | | | |
| | amine | | | |
| 39 | [2-(2,3-Dimethoxy-phenyl)-7-methyl- | 58 | 387.48 | 388.4 |
| | imidazo[1,2-a]pyridin-3-yl]-(2,6-dimethyl- | | | |
| | phenyl)-amine | | | |
| 40 | (2,7-Dimethyl-imidazo[1,2-a]pyridin-3-yl)- | 61 | 273.42 | 274.3 |
| | (1,1,3,3-tetramethyl-butyl)-amine | | | |
| 41 | [2-(5-[1,3]Dioxolan-2-yl-furan-2-yl)-7- | 57 | 397.51 | 398.4 |
| | methyl-imidazo[1,2-a]pyridin-3-yl]-(1,1,3,3- | | | |
| | tetramethyl-butyl)-amine | | | |
| 42 | [2-(3-Bromo-thiophen-2-yl)-5,7-dimethyl- | 55 | 426.38 | 426.3/428.2 |
| | imidazo[1,2-a]pyridin-3-yl]-(2,6-dimethyl- | | | |
| | phenyl)-amine | | | |
| 43 | (2,6-Dimethyl-phenyl)-[2-(2-fluoro-phenyl)- | 71 | 345.42 | 346.3 |
| | 7-methyl-imidazo[1,2-a]pyridin-3-yl]-amine | | | |
| 44 | (2-Cyclohexyl-5,7-dimethyl-imidazo[1,2-a]- | 71 | 355.56 | 356.3 |
| | pyridin-3-yl)-(1,1,3,3-tetramethyl-butyl)- | | | |
| | amine | | | |
| 45 | [6-(2-Furan-2-yl-5,7-dimethyl-imidazo[1,2- | 54 | 337.44 | 337.4 |
| | a]pyridin-3-ylamino)-hexyl]-methylidyne- | | | |
| | ammonium | | | |
| 46 | (7-Methyl-2-o-tolyl-imidazo[1,2-a]pyridin-3- | 59 | 349.52 | 350.4 |
| | yl)-(1,1,3,3-tetramethyl-butyl)-amine | | | |
| 47 | [2-(2,3-Dichloro-phenyl)-7-methyl- | 69 | 396.32 | 396.3/398.3 |
| | imidazo[1,2-a]pyridin-3-yl]-(2,6-dimethyl- | | | |
| | phenyl)-amine | | | |
| 48 | [2-(2,3-Dimethoxy-phenyl)-5,7-dimethyl- | 68 | 401.5 | 402.3 |
| | imidazo[1,2-a]pyridin-3-yl]-(2,6-dimethyl- | | | |
| | phenyl)-amine | | | |
| 49 | Butyl-[2-(2,3-dimethoxy-phenyl)-5,7- | 67 | 353.46 | 354.3 |
| | dimethyl-imidazo[1,2-a]pyridin-3-yl]-amine | | | |
| 50 | Methylidyne-[6-(7-methyl-2-p-tolyl- | 78 | 347.48 | 347.5 |
| | imidazo[1,2-a]pyridin-3-ylamino)-hexyl]- | | | |
| | ammonium | | | |
| 51 | Tert-butyl-[5,7-dimethyl-2-(5-nitro-furan-2- | 59 | 328.37 | 329.4 |
| | yl)-imidazo[1,2-a]pyridin-3-yl]-amine | | | |
| 52 | Acetic acid 5-(3-cyclohexylamino-5,7- | 64 | 381.47 | 382.4 |
| | dimethyl-imidazo[1,2-a]pyridin-2-yl)-furan- | | | |
| | 2-yl methyl ester | | | |
| 53 | [2-(2-Methoxy-phenyl)-5,7-dimethyl- | 61 | 379.54 | 380.3 |
| | imidazo[1,2-a]pyridin-3-yl]-(1,1,3,3- | | | |
| | tetramethyl-butyl)-amine | | | |
| 54 | [2-(3,4-Dimethoxy-phenyl)-7-methyl- | 64 | 387.48 | 388.3 |
| | imidazo[1,2-a]pyridin-3-yl]-(2,6-dimethyl- | | | |
| | phenyl)-amine | | | |
| 55 | 3-(3-Butylamino-7-methyl-imidazo[1,2-a]- | 63 | 295.38 | 296.2 |
| | pyridin-2-yl)-phenol | | | |
| 56 | (2-Benzofuran-2-yl-7-methyl-imidazo[1,2- | 60 | 375.51 | 376.4 |
| | a]pyridin-3-yl)-(1,1,3,3-tetramethyl-butyl)- | | | |
| | amine | | | |
| 57 | (2-Benzofuran-2-yl-5,7-dimethyl- | 57 | 381.47 | 382.4 |
| | imidazo[1,2-a]pyridin-3-yl)-(2,6-dimethyl- | | | |
| | phenyl)-amine | | | |
| 58 | Acetic acid 5-(3-cyclohexylamino-7-methyl- | 64 | 367.44 | 368.4 |
| | imidazo[1,2-a]pyridin-2-yl)-furan-2-yl methyl | | | |
| | ester | | | |
| 59 | [6-(5,7-Dimethyl-2-pyridin-3-yl-imidazo[1,2- | 57 | 348.47 | 348.4 |
| | a]pyridin-3-ylamino)-hexyl]-methylidyne- | | | |
| | ammonium | | | |
| 60 | Butyl-[2-(2-methoxy-phenyl)-7-methyl- | 52 | 309.41 | 310.3 |
| | imidazo[1,2-a]pyridin-3-yl]-amine | | | |
| 61 | {6-[2-(2-Methoxy-phenyl)-5,7-dimethyl- | 73 | 377.51 | 377.3 |
| | imidazo[1,2-a]pyridin-3-ylamino]-hexyl}- | | | |
| | methylidyne-ammonium | | | |
| 62 | {5-[5,7-Dimethyl-3-(1,1,3,3-tetramethyl- | 55 | 369.5 | 370.4 |
| | butylamino)-imidazo[1,2-a]pyridin-2-yl]- | | | |
| | furan-2-yl}-methanol | | | |
| 63 | (7-Methyl-2-naphthalen-1-yl-imidazo[1,2-a]- | 52 | 385.55 | 386.3 |
| | pyridin-3-yl)-(1,1,3,3-tetramethyl-butyl)- | | | |
| | amine | | | |
| 64 | [5-(3-Tert-butylamino-7-methyl-imidazo[1,2- | 52 | 299.37 | 300.3 |
| | a]pyridin-2-yl)-furan-2-yl]-methanol | | | |
| 65 | Tert-butyl-[2-(5-[1,3]dioxolan-2-yl-furan-2- | 62 | 341.41 | 342.4 |
| | yl)-7-methyl-imidazo[1,2-a]pyridin-3-yl]- | | | |
| | amine | | | |
| 66 | (2-Benzofuran-2-yl-5,7-dimethyl- | 53 | 389.54 | 390.4 |
| | imidazo[1,2-a]pyridin-3-yl)-(1,1,3,3- | | | |
| | tetramethyl-butyl)-amine | | | |
| 67 | 5-(3-Tert-butylamino-5,7-dimethyl- | 56 | 327.38 | 328.3 |
| | imidazo[1,2-a]pyridin-2-yl)-furan-2- | | | |
| | carboxylic acid | | | |
| 68 | Tert-butyl-(2-furan-2-yl-7-methyl- | 57 | 269.34 | 270.4 |
| | imidazo[1,2-a]pyridin-3-yl)-amine | | | |
| 69 | Cyclohexyl-(2,7-dimethyl-imidazo[1,2-a]- | 55 | 243.35 | 244.4 |
| | pyridin-3-yl)-amine | | | |
| 70 | [2-(2,3-Dichlorophenyl)-8-methyl- | 52 | 404.38 | 404.3/406.2 |
| | imidazo[1,2-a]pyridin-3-yl]-(1,1,3,3- | | | |
| | tetramethyl-butyl)-amine | | | |
| 71 | (7-Methyl-2-p-tolyl-imidazo[1,2-a]pyridin-3- | 58 | 349.52 | 350.3 |
| | yl)-(1,1,3,3-tetramethyl-butyl)-amine | | | |
| 72 | (2,6-Dimethyl-phenyl)-[2-(2-methoxy- | 53 | 357.45 | 358.3 |
| | phenyl)-7-methyl-imidazo[1,2-a]pyridin-3- | | | |
| | yl]-amine | | | |
| 73 | 3-(3-Butylamino-5,7-dimethyl-imidazo[1,2- | 61 | 309.41 | 310.2 |
| | a]pyridin-2-yl)-phenol | | | |
| 74 | Butyl-[2-(2,3-dimethoxy-phenyl)-7-methyl- | 62 | 339.43 | 340.4 |
| | imidazo[1,2-a]pyridin-3-yl]-amine | | | |
| 75 | {6-[5,7-Dimethyl-2-(2-trifluorO-methyl- | 56 | 415.48 | 415.3 |
| | phenyl)-imidazo[1,2-a]pyridin-3-ylamino]- | | | |
| | hexyl}-methylidyne-ammonium | | | |
| 76 | Tert-butyl-(7-methyl-2-pyridin-2-yl- | 67 | 280.37 | 281.3 |
| | imidazo[1,2-a]pyridin-3-yl)-amine | | | |
| 77 | Cyclohexyl-(2-cyclohexyl-5,7-dimethyl- | 63 | 325.49 | 326.4 |
| | imidazo[1,2-a]pyridin-3-yl)-amine | | | |
| 78 | [2-(2,3-Dichlorophenyl)-5,7-dimethyl- | 61 | 410.34 | 410.3/412.2 |
| | imidazo[1,2-a]pyridin-3-yl]-(2,6-dimethyl- | | | |
| | phenyl)-amine | | | |
| 79 | (2,6-Dimethyl-phenyl)-[2-(2-methoxy- | 66 | 371.48 | 372.3 |
| | phenyl)-5,7-dimethyl-imidazo[1,2-a]pyridin- | | | |
| | 3-yl]-amine | | | |
| 80 | {2-[5-(2-Chlorophenyl)-furan-2-yl]-7-methyl- | 52 | 435.99 | 436.4/437.2/ |
| | imidazo[1,2-a]pyridin-3-yl}-(1,1,3,3- | | | 438.4 |
| | tetramethyl-butyl)-amine | | | |
| 81 | 5-[7-Methyl-3-(1,1,3,3-tetramethyl- | 60 | 369.46 | 370.4 |
| | butylamino)-imidazo[1,2-a]pyridin-2-yl]- | | | |
| | furan-2-carboxylic acid | | | |
| 82 | Cyclohexyl-[2-(2-methoxy-phenyl)-8- | 62 | 335.45 | 336.4 |
| | methyl-imidazo[1,2-a]pyridin-3-yl]-amine | | | |
| 83 | 3-[7-Methyl-3-(1,1,3,3-tetramethyl- | 60 | 351.49 | 352.4 |
| | butylamino)-imidazo[1,2-a]pyridin-2-yl]- | | | |
| | phenol | | | |
| 84 | [2-(2,3-Dichlorophenyl)-5,7-dimethyl- | 54 | 418.41 | 418.2/420.2 |
| | imidazo[1,2-a]pyridin-3-yl]-(1,1,3,3- | | | |
| | tetramethyl-butyl)-amine | | | |
| 85 | [2-(2,4-Dichlorophenyl)-7-methyl- | 61 | 404.38 | 404.4/406.2 |
| | imidazo[1,2-a]pyridin-3-yl]-(1,1,3,3- | | | |
| | tetramethyl-butyl)-amine | | | |
| 86 | [2-(5-Bromofuran-2-yl)-7-methyl- | 54 | 404.35 | 404.3/406.3 |
| | imidazo[1,2-a]pyridin-3-yl]-(1,1,3,3- | | | |
| | tetramethyl-butyl)-amine | | | |
| 87 | 5-(3-Cyclohexylamino-5,7-dimethyl- | 54 | 353.42 | 354.4 |
| | imidazo[1,2-a]pyridin-2-yl)-furan-2- | | | |
| | carboxylic acid | | | |
| 88 | [6-(2-Cyclohexyl-5,7-dimethyl-imidazo[1,2- | 57 | 353.53 | 353.4 |
| | a]pyridin-3-ylamino)-hexyl]-methylidyne- | | | |
| | ammonium | | | |
| 89 | [2-(2,4-Dichlorophenyl)-5,7-dimethyl- | 68 | 418.41 | 418.3/420.2 |
| | imidazo[1,2-a]pyridin-3-yl]-(1,1,3,3- | | | |
| | tetramethyl-butyl)-amine | | | |
| 90 | (2-benzofuran-2-yl-7-methyl-imidazo[1,2-a]- | 53 | 367.45 | 368.4 |
| | pyridin-3-yl)-(2,6-dimethyl-phenyl)-amine | | | |
| 91 | 5-(3-Cyclohexylamino-7-methyl- | 64 | 339.39 | 340.4 |
| | imidazo[1,2-a]pyridin-2-yl)-furan-2- | | | |
| | carboxylic acid | | | |
| 92 | {6-[2-(2-Bromophenyl)-5,7-dimethyl- | 60 | 426.38 | 425.3/427.2 |
| | imidazo[1,2-a]pyridin-3-ylamino]-hexyl}- | | | |
| | methylidyne-ammonium | | | |
| 93 | Tert-butyl-(2-cyclohexyl-7-methyl- | 52 | 285.43 | 286.4 |
| | imidazo[1,2-a]pyridin-3-yl)-amine | | | |
| 94 | Tert-butyl-(2,7-dimethyl-imidazo[1,2-a]- | 65 | 217.31 | 218.2 |
| | pyridin-3-yl)-amine | | | |
| 95 | (5,7-Dimethyl-2-p-tolyl-imidazo[1,2-a]- | 70 | 363.54 | 364.4 |
| | pyridin-3-yl)-(1,1,3,3-tetramethyl-butyl)- | | | |
| | amine | | | |
| 96 | [2-(2,3-Dichlorophenyl)-8-methyl- | 55 | 396.32 | 396.3/398.3 |
| | imidazo[1,2-a]pyridin-3-yl]-(2,6-dimethyl- | | | |
| | phenyl)-amine | | | |
| 97 | Methylidyne-[6-(7-methyl-2-o-tolyl- | 62 | 347.48 | 347.5 |
| | imidazo[1,2-a]pyridin-3-ylamino)-hexyl]- | | | |
| | ammonium | | | |
| 98 | {2-[5-(3-Chlorophenyl)-furan-2-yl]-7-methyl- | 62 | 435.99 | 436.4/438.3 |
| | imidazo[1,2-a]pyridin-3-yl}-(1,1,3,3- | | | |
| | tetramethyl-butyl)-amine | | | |
| 99 | Cyclohexyl-[7-methyl-2-(5-nitro-furan-2-yl)- | 66 | 340.38 | 341.4 |
| | imidazo[1,2-a]pyridin-3-yl]-amine | | | |
| 100 | [2-(2-Bromophenyl)-7-methyl-imidazo[1,2- | 50 | 384.32 | 384.4/386.4/ |
| | a]pyridin-3-yl]-cyclohexyl-amine | | | 387.3 |
| 101 | [2-(2-Methoxy-phenyl)-7-methyl- | 58 | 365.52 | 366.4 |
| | imidazo[1,2-a]pyridin-3-yl]-(1,1,3,3- | | | |
| | tetramethyl-butyl)-amine | | | |
| 102 | {5-[7-Methyl-3-(1,1,33-tetramethyl- | 59 | 355.48 | 356.5 |
| | butylamino)-imidazo[1,2-a]pyridin-2-yl]- | | | |
| | furan-2-yl}-methanol | | | |
| 103 | (6-{2-[5-(2-Chlorophenyl)-furan-2-yl]-5- | 50 | 433.96 | 433.4/435.4 |
| | methyl-imidazo[1,2-a]pyridin-3-ylamino)- | | | |
| | hexyl)-methylidyne-ammonium | | | |
| 104 | Cyclohexyl-[5,7-dimethyl-2-(5-nitro-furan-2- | 63 | 354.4 | 355.4 |
| | yl)-imidazo[1,2-a]pyridin-3-yl]-amine | | | |
| 105 | Cyclohexyl-[2-(4,5-dimethyl-furan-2-yl)-7- | 65 | 323.43 | 324.4 |
| | methyl-imidazo[1,2-a]pyridin-3-yl]-amine | | | |
| 106 | [6-(5,7-Dimethyl-2-o-tolyl-imidazo[1,2-a]- | 60 | 361.51 | 361.4 |
| | pyridin-3-ylamino)-hexyl]-methylidyne- | | | |
| | ammonium | | | |
| 107 | Methylidyne-[6-(7-methyl-2-pyridin-2-yl- | 50 | 334.44 | 334.4 |
| | imidazo[1,2-a]pyridin-3-ylamino)-hexyl]- | | | |
| | ammonium | | | |
| 108 | (2-(2,3-Dimethoxy-phenyl)-5,7-dimethyl- | 73 | 409.57 | 410.4 |
| | imidazo[1,2-a]pyridin-3-yl]-(1,1,3,3- | | | |
| | tetramethyl-butyl)-amine | | | |
| 109 | {6-[2-(3,4-Dimethoxy-phenyl)-7-methyl- | 51 | 393.51 | 393.4 |
| | imidazo[1,2-a]pyridin-3-ylamino]-hexyl}- | | | |
| | methylidyne-ammonium | | | |
| 110 | 5-(3-Tert-butylamino-7-methyl-imidazo[1,2- | 61 | 329.42 | 330.3 |
| | a]pyridin-2-yl)-thiophene-2-carboxylic acid | | | |
| 111 | Cyclohexyl-(8-methyl-2-pyridin-4-yl- | 52 | 306.41 | 307.4 |
| | imidazo[1,2-a]pyridin-3-yl)-amine | | | |
| 112 | [2-(2,3-Dichloro-phenyl)-6-methyl- | 52 | 404.38 | 404.3/406.3 |
| | imidazo[1,2-a]pyridin-3-yl]-(1,1,3,3- | | | |
| | tetramethyl-butyl)-amine | | | |
| 113 | 5-(3-Butylamino-imidazo[1,2-a]pyrazin-2- | 54 | 316.38 | 317.3 |
| | yl)-thiophene-2-carboxylic acid | | | |
| 114 | Cyclohexyl-(5,7-dimethyt-2-pyridin-2-yl- | 53 | 320.43 | 321.4 |
| | imidazo[1,2-a]pyridin-3-yl)-amine | | | |
| 115 | (2-Benzofuran-2-yl-8-methyl-imidazo[1,2- | 59 | 375.51 | 376.4 |
| | a]pyridin-3-yl)-(1,1,3,3-tetramethyl-butyl)- | | | |
| | amine | | | |
| 116 | {6-[2-(2-Fluoro-phenyl)-5,7-dimethyl- | 60 | 365.47 | 365.3 |
| | imidazo[1,2-a]pyridin-3-ylamino]-hexyl}- | | | |
| | methylidyne-ammonium | | | |
| 117 | [2-(2,3-Dimethoxy-phenyl)-8-methyl- | 52 | 395.54 | 396.3 |
| | imidazo[1,2-a]pyridin-3-yl]-(1,1,3,3- | | | |
| | tetramethyl-butyl)-amine | | | |
| 118 | Methylidyne-[6-(7-methyl-2-phenanthren-9- | 56 | 433.57 | 433.5 |
| | yl-imidazo[1,2-a]pyridin-3-ylamino)-hexyl]- | | | |
| | ammonium | | | |
| 119 | 5-(3-Tert-butylamino-imidazo[1,2-a]pyrazin- | 56 | 316.38 | 317.4 |
| | 2-yl)-thiophene-2-carboxylic acid | | | |
| 120 | Tert-butyl-(8-methyl-2-pyridin-2-yl- | 56 | 280.37 | 281.2 |
| | imidazo[1,2-a]pyridin-3-yl)-amine | | | |
| 121 | Cyclohexyl-(2-furan-2-yl-8-methyl- | 53 | 295.38 | 296.4 |
| | imidazo[1,2-a]pyridin-3-yl)-amine | | | |
| 122 | Methylidyne-[6-(7-methyl-2-naphthalen-1- | 53 | 383.51 | 383.4 |
| | yl-imidazo[1,2-a]pyridin-3-ylamino)-hexyl]- | | | |
| | ammonium | | | |
| 123 | Tert-butyl-(2-cyclohexyl-8-methyl- | 53 | 285.43 | 286.4 |
| | imidazo[1,2-a]pyridin-3-yl)-amine | | | |
| 124 | (6-Methyl-2-pyridin-4-yl-imidazo[1,2-a]- | 68 | 336.48 | 337.4 |
| | pyridin-3-yl)-(1,1,3,3-tetramethyl-butyl)- | | | |
| | amine | | | |
| 125 | Tert-butyl-(6-methyl-2-pyridin-3-yl- | 51 | 280.37 | 281.3 |
| | imidazo[1,2-a]pyridin-3-yl)-amine | | | |
| 126 | (7-Methyl-2-pyridin-3-yl-imidazo[1,2-a]- | 50 | 337.47 | 338.4 |
| | pyrimidin-3-yl)-(1,1,3,3-tetramethyl-butyl)- | | | |
| | amine | | | |
| 127 | 5-(3-Tert-butylamino-6-methyl-imidazo[1,2- | 52 | 329.42 | 330.2 |
| | a]pyridin-2-yl)-thiophene-2-carboxylic acid | | | |
| 128 | [6-(5,7-Dimethyl-2-naphthalen-1-yl- | 63 | 397.54 | 397.3 |
| | imidazo[1,2-a]pyridin-3-ylamino)-hexyl]- | | | |
| | methylidyne-ammonium | | | |
| 129 | 3-[3-(2,6-Dimethyl-phenylamino)-5,7- | 61 | 357.45 | 358.3 |
| | dimethyl-imidazo[1,2-a]pyridin-2-yl]-phenol | | | |
| 130 | (2,6-Dimethyl-phenyl)-(8-methyl-2-o-tolyl- | 58 | 341.45 | 342.3 |
| | imidazo[1,2-a]pyridin-3-yl)-amine | | | |
| 131 | {6-[2-(3-Hydroxy-phenyl)-8-methyl- | 55 | 349.45 | 349.4 |
| | imidazo[1,2-a]pyridin-3-ylamino]-hexyl}- | | | |
| | methylidyne-ammonium | | | |
| 132 | {5-[3-(2,6-Dimethyl-phenylamino)-7-methyl- | 52 | 348.4 | 349.4 |
| | imidazo[1,2-a]pyrimidin-2-yl]-furan-2-yl}- | | | |
| | methanol | | | |
| 133 | (8-Methyl-2-p-tolyl-imidazo[1,2-a]pyridin-3- | 52 | 349.52 | 350.3 |
| | yl)-(1,1,3,3-tetramethyl-butyl)-amine | | | |
| 134 | [2-(2,4-Dichlorophenyl)-6-methyl- | 56 | 396.32 | 396.2/398.2 |
| | imidazo[1,2-a]pyridin-3-yl]-(2,6-dimethyl- | | | |
| | phenyl)-amine | | | |
| 135 | Butyl-[2-(2,4-dichloro-phenyl)-6-methyl- | 50 | 348.27 | 348.3/350.2 |
| | imidazo[1,2-a]pyridin-3-yl]-amine | | | |
| 136 | Butyl-[2-(4-dimethylamino-naphthalen-1-yl)- | 56 | 359.47 | 360.5 |
| | imidazo[1,2-a]pyrazin-3-yl]-amine | | | |
| 137 | {6-[2-(2-Bromo-phenyl)-7-methyl- | 52 | 412.35 | 411.3/414.2 |
| | imidazo[1,2-a]pyridin-3-ylamino]-hexyl}- | | | |
| | methylidyne-ammonium | | | |
| 138 | Butyl-[2-(2-methoxy-phenyl)-6-methyl- | 52 | 309.41 | 310.3 |
| | imidazo[1,2-a]pyridin-3-yl]-amine | | | |
| 139 | (2-Cyclohexyl-8-methyl-imidazo[1,2-a]- | 51 | 341.54 | 342.5 |
| | pyridin-3-yl)-(1,1,3,3-tetramethyl-butyl)- | | | |
| | amine | | | |
| 140 | Cyclohexyl-(7-methyl-2-pyridin-2-yl- | 55 | 306.41 | 307.3 |
| | imidazo[1,2-a]pyridin-3-yl)-amine | | | |
| 141 | Cyclohexyl-(2-furan-2-yl-7-methyl- | 64 | 295.38 | 296.4 |
| | imidazo[1,2-a]pyridin-3-yl)-amine | | | |
| 142 | (2-Cyclohexyl-5,7-dimethyl-imidazo[1,2-a]- | 59 | 315.41 | 316.4 |
| | pyridin-3-ylamino)-acetic acid methyl ester | | | |
| 313 | [7-Ethyl-2-(5-nitro-furan-2-yl)-imidazo[1,2- | 32 | 392.42 | 393.9 |
| | a]pyridin-3-yl]-(2-methoxy-benzyl)-amine | | | |
| 314 | (2-Chlorobenzyl)-[7-ethyl-2-(5-nitro-furan-2- | 47 | 396.83 | 387.3 |
| | yl)-imidazo[1,2-a]pyridin-3-yl]-amine | | | |
| 315 | [7-Ethyl-2-(5-methyl-furan-2-yl)- | 32 | 361.44 | 362.3 |
| | imidazo[1,2-a]pyridin-3-yl]-(2-methoxy- | | | |
| | benzyl)-amine | | | |
| 316 | (2-Chloro-benzyl)-(7-ethyl-2-furan-2-yl- | 52 | 351.84 | 352.4 |
| | imidazo[1,2-a]pyridin-3-yl)-amine | | | |
| 317 | (3-Chloro-4-fluoro-phenyl)-[7-ethyl-2-(5- | 59 | 369.84 | 370.5 |
| | methyl-furan-2-yl)-imidazo[1,2-a]pyridin-3- | | | |
| | yl]-amine | | | |
| 318 | (2-Benzofuran-2-yl-7-ethyl-imidazo[1,2-a]- | 52 | 405.86 | 406 |
| | pyridin-3-yl)-(3-chloro-4-fluoro-phenyl)- | | | |
| | amine | | | |
| 319 | (2-Benzofuran-2-yl-7-ethyl-imidazo[1,2-a]- | 53 | 387.87 | 388 |
| | pyridin-3-yl)-(3-chloro-phenyl)-amine | | | |
| 320 | (3-Chloro-4-fluorophenyl)-{2-[5-(3-chloro- | 52 | 466.34 | 466/468 |
| | phenyl)-furan-2-yl]-7-ethyl-imidazo[1,2-a]- | | | |
| | pyridin-3-yl}-amine | | | |
| 321 | (3-Chloro-4-fluorophenyl)-{2-[5-(2- | 50 | 466.34 | 466/468 |
| | chlorophenyl)-furan-2-yl]-7-ethyl- | | | |
| | imidazo[1,2-a]pyridin-3-yl}-amine | | | |
| 322 | (3-Chloro-4-fluorophenyl)-[2-(4,5-dimethyl- | 49 | 383.85 | 384 |
| | furan-2-yl)-7-ethyl-imidazo[1,2-a]pyridin-3- | | | |
| | yl]-amine | | | |

As comparison example, 7-nitroindazole was tested in the NOS assay with an inhibition (10 μM) of 50%.

General Working Procedure 2 (GWP 2):

About 0.05 mmol of the educt having the general formula I obtained according to GWP 1, in solid form, was placed in a round-bottomed test tube equipped with a stirrer. 2 ml of dichloromethane were added at 18° C., with stirring. 4 equivalents of acetyl chloride (0.2 M solution in dichloromethane) were added to the solution, and stirring was carried out for 4 hours.

The stirrer was then removed and the organic solutions were concentrated to dryness in a vacuum centrifuge at 40-50° C. An ESI-MS was recorded for characterisation purposes.

Examples 143-291 prepared according to GWP 2 were tested in the HTS-NOS assay (HTS) in an automated manner; the results are shown in Table 3.

TABLE 3

| Example | | HTS-NOS assay: % | Weight | Weight |
|---|---|---|---|---|
| No. | Compound | inhibition (10 μM) | calc. | found |
| 143 | N-(2-Furan-2-yl-5,7-dimethyl-imidazo[1,2-a]- | 68 | 381.51 | (M-acetyl) |
| | pyridin-3-yl)-N-(1,1,3,3-tetramethyl-butyl)- | | | 340.5 |
| | acetamide | | | |
| 144 | N-Tert-butyl-N-(7-methyl-2-pyridin-3-yl- | 59 | 322.41 | (M-acetyl) |
| | imidazo[1,2-a]pyridin-3-yl)-acetamide | | | 281.4 |
| 145 | N-Tert-butyl-N-(2-furan-2-yl-5,7-dimethyl- | 66 | 325.41 | (M-acetyl) |
| | imidazo[1,2-a]pyridin-3-yl)-acetamide | | | 284.3 |
| 146 | N-(5,7-Dimethyl-2-pyridin-4-yl-imidazo[1,2- | 59 | 392.54 | (M-acetyl) |
| | a]pyridin-3-yl)-N-(1,1,3,3-tetramethyl-butyl)- | | | 351.4 |
| | acetamide | | | |
| 147 | N-(5,7-Dimethyl-2-pyridin-3-yl-imidazo[1,2- | 68 | 392.54 | (M-acetyl) |
| | a]pyridin-3-yl)-N-(1,1,3,3-tetramethyl-butyl)- | | | 351.5 |
| | acetamide | | | |
| 148 | N-(2,6-Dimethyl-phenyl)-N-(5,7-dimethyl-2- | 57 | 397.52 | (M-acetyl) |
| | o-tolyl-imidazo[1,2-a]pyridin-3-yl)-acetamide | | | 356.4 |
| 149 | N-(2-Furan-2-yl-7-methyl-imidazo[1,2-a]- | 55 | 367.49 | (M-acetyl) |
| | pyridin-3-yl)-N-(1,1,3,3-tetramethyl-butyl)- | | | 326.5 |
| | acetamide | | | |
| 150 | N-(1,1,3,3-Tetramethyl-butyl)-N-(2,5,7- | 60 | 329.48 | (M-acetyl) |
| | trimethyl-imidazo[1,2-a]pyridin-3-yl)- | | | 288.5 |
| | acetamide | | | |
| 151 | N-Cyclohexyl-N-(7-methyl-2-thiophen-2-yl- | 65 | 353.48 | (M-acetyl) |
| | imidazo[1,2-a]pyridin-3-yl)-acetamide | | | 312.5 |
| 152 | N-Tert-butyl-N-(2,5,7-trimethyl-imidazo[1,2- | 66 | 273.37 | (M-acetyl) |
| | a]pyridin-3-yl)-acetamide | | | 232.3 |
| 153 | 5-[3-(Acetyl-tert-butyl-amino)-imidazo[1,2-a]- | 78 | 358.41 | (M-acetyl) |
| | pyrazin-2-yl]-thiophene-2-carboxylic acid | | | 317.5 |
| 154 | 5-{3-[Acetyl-(1,1,3,3-tetramethyl-butyl)- | 60 | 425.52 | (M-acetyl) |
| | amino]-5,7-dimethyl-imidazo[1,2-a]pyridin-2- | | | 384.6 |
| | yl}-furan-2-carboxylic acid | | | |
| 155 | N-[2-(5-Hydroxymethyl-furan-2-yl)-5,7- | 50 | 411.54 | 370.4; (M- |
| | dimethyl-imidazo[1,2-a]pyridin-3-yl]-N- | | | acetyl) |
| | (1,1,3,3-tetramethyl-butyl)-acetamide | | | 412.4 |
| 156 | N-[2-(3-Bromo-thiophen-2-yl)-5,7-dimethyl- | 74 | 468.42 | (M-acetyl) |
| | imidazo[1,2-a]pyridin-3-yl]-N-(2,6-dimethyl- | | | 426.3/428.3 |
| | phenyl)-acetamide | | | |
| 157 | N-Tert-butyl-N-(2-cyclohexyl-5,7-dimethyl- | 63 | 341.49 | (M-acetyl) |
| | imidazo[1,2-a]pyridin-3-yl)-acetamide | | | 300.4 |
| 158 | Acetic acid 5-[3-(acetyl-cyclohexyl-amino)- | 54 | 423.51 | (M-acetyl) |
| | 5,7-dimethyl-imidazo[1,2-a]pyridin-2-yl]- | | | 382.4 |
| | furan-2-yl methyl ester | | | |
| 159 | {6-[Acetyl-(2-furan-2-yl-5,7-dimethyl- | 61 | 379.48 | (M-acetyl) |
| | imidazo[1,2-a]pyridin-3-yl)-amino]-hexyl}- | | | 337.4 |
| | methylidyne-ammonium | | | |
| 160 | N-[2-(2,3-Dichloro-phenyl)-7-methyl- | 55 | 438.35 | (M-acetyl) |
| | imidazo[1,2-a]pyridin-3-yl]-N-(2,6-dimethyl- | | | 396.4/398.3 |
| | phenyl)-acetamide | | | |
| 161 | N-[2-(3-Bromo-thiophen-2-yl)-imidazo[1,2-a]- | 51 | 418.36 | (M-acetyl) |
| | pyridin-3-yl]-N-cyclohexyl-acetamide | | | 376.5/378.4 |
| 162 | N-(5,7-Dimethyl-2-p-tolyl-imidazo[1,2-a]- | 67 | 405.58 | (M-acetyl) |
| | pyridin-3-yl)-N-(1,1,3,3-tetramethyl-butyl)- | | | 364.4 |
| | acetamide | | | |
| 163 | N-Cyclohexyl-N-(7-methyl-2-pyridin-4-yl- | 63 | 348.44 | (M-acetyl) |
| | imidazo[1,2-a]pyridin-3-yl)-acetamide | | | 307.4 |
| 164 | N-Cyclohexyl-N-[7-methyl-2-(2-trifluorO- | 61 | 415.45 | (M-acetyl) |
| | methyl-phenyl)-imidazo[1,2-a]pyridin-3-yl]- | | | 374.5 |
| | acetamide | | | |
| 165 | N-(6,8-Dibromo-2-furan-2-yl-imidazo[1,2-a]- | 67 | 511.26 | (M-acetyl) |
| | pyridin-3-yl)-N-(1,1,3,3-tetramethyl-butyl)- | | | 468.3/470.2/ |
| | acetamide | | | 472.2 |
| 166 | N-(7-Methyl-2-pyridin-4-yl-imidazo[1,2-a]- | 61 | 378.51 | (M-acetyl) |
| | pyridin-3-yl)-N-(1,1,3,3-tetramethyl-butyl)- | | | 337.4 |
| | acetamide | | | |
| 167 | Acetic acid 5-[3-(acetyl-cyclohexyl-amino)-7- | 68 | 409.48 | (M-acetyl) |
| | methyl-imidazo[1,2-a]pyridin-2-yl]-furan-2-yl | | | 368.5 |
| | methyl ester | | | |
| 168 | N-(7-Methyl-2-pyridin-3-yl-imidazo[1,2-a]- | 56 | 378.51 | (M-acetyl) |
| | pyridin-3-yl)-N-(1,1,3,3-tetramethyl-butyl)- | | | 337.4 |
| | acetamide | | | |
| 169 | N-[2-(2,3-Dichloro-phenyl)-5,7-dimethyl- | 57 | 452.38 | (M-acetyl) |
| | imidazo[1,2-a]pyridin-3-yl]-N-(2,6-dimethyl- | | | 410.3/412.2/ |
| | phenyl)-acetamide | | | 413.2 |
| 170 | N-Cyclohexyl-N-[5,7-dimethyl-2-(5-methyl- | 65 | 365.47 | (M-acetyl) |
| | furan-2-yl)-imidazo[1,2-a]pyridin-3-yl]- | | | 324.4 |
| | acetamide | | | |
| 171 | N-Butyl-N-[2-(2,3-dimethoxy-phenyl)-5,7- | 63 | 395.5 | (M-acetyl) |
| | dimethyl-imidazo[1,2-a]pyridin-3-yl]- | | | 354.4 |
| | acetamide | | | |
| 172 | N-[2-(2-Methoxy-phenyl)-5,7-dimethyl- | 62 | 421.58 | (M-acetyl) |
| | imidazo[1,2-a]pyridin-3-yl]-N-(1,1,3,3- | | | 380.4 |
| | tetramethyl-butyl)-acetamide | | | |
| 173 | N-Cyclohexyl-N-[5,7-dimethyl-2-(5-nitro | 67 | 396.44 | (M-acetyl) |
| | furan-2-yl)-imidazo[1,2-a]pyridin-3-yl]- | | | 355.4 |
| | acetamide | | | |
| 174 | Acetyl-(2,7-dimethyl-imidazo[1,2-a]pyridin-3- | 68 | 275.3 | (M-acetyl) |
| | yl)-amino]-acetic acid methyl ester | | | 234.4 |
| 175 | N-Cyclohexyl-N-(2,5,7-trimethyl-imidazo[1,2- | 52 | 299.41 | (M-acetyl) |
| | a]pyridin-3-yl)-acetamide | | | 258.4 |
| 176 | 5-{3-[Acetyl-(1,1,3,3-tetramethyl-butyl)- | 50 | 413.53 | (M-acetyl) |
| | amino]-imidazo[1,2-a]pyridin-2-yl}- | | | 372.5 |
| | thiophene-2-carboxylic acid | | | |
| 177 | N-(2-(2,4-Dichloro-phenyl)5,7-dimethyl- | 67 | 460.44 | (M-acetyl) |
| | imidazo[1,2-a]pyridin-3-yl]-N-(1,1,3,3- | | | 418.3/420.3/ |
| | tetramethyl-butyl)-acetamide | | | 421.2 |
| 178 | N-Cyclohexyl-N-[7-methyl-2-(5-nitro-furan-2- | 64 | 382.41 | (M-acetyl) |
| | yl)-imidazo[1,2-a]pyridin-3-yl]-acetamide | | | 341.5 |
| 179 | N-(2-Tert-butyl-5,7-dimethyl-imidazo[1,2-a]- | 60 | 363.5 | (M-acetyl) |
| | pyridin-3-yl)-N-(2,6-dimethyl-phenyl)- | | | 322.4 |
| | acetamide | | | |
| 180 | N-(2,6-dimethyl-phenyl)-N-[2-(2-methoxy- | 54 | 413.52 | (M-acetyl) |
| | phenyl)-5,7-dimethyl-imidazo[1,2-a]pyridin- | | | 372.4 |
| | 3-yl]-acetamide | | | |
| 181 | N-[2-(3-Hydroxy-phenyl)-5,7-dimethyl- | 60 | 407.55 | 408.3: (M- |
| | imidazo[1,2-a]pyridin-3-yl]-N-(1,1,3,3- | | | acetyl) |
| | tetramethyl-butyl)-acetamide | | | 366.4 |
| 182 | N-(2,6-dimethyl-phenyl)-N-[2-(2-fluoro- | 53 | 401.48 | (M-acetyl) |
| | phenyl)-5,7-dimethyl-imidazo[1,2-a]pyridin- | | | 360.4 |
| | 3-yl]-acetamide | | | |
| 183 | 5-[3-(Acetyl-tert-butyl-amino)-5-methyl- | 50 | 371.45 | (M-acetyl) |
| | imidazo[1,2-a]pyridin-2-yl]-thiophene-2- | | | 330.4 |
| | carboxylic acid | | | |
| 184 | N-(2,6-Dimethyl-phenyl)-N-[2-(2-methoxy- | 51 | 399.49 | (M-acetyl) |
| | phenyl)-7-methyl-imidazo[1,2-a]pyridin-3-yl]- | | | 358.5 |
| | acetamide | | | |
| 185 | N-(7-Methyl-2-p-tolyl-imidazo[1,2-a]pyridin- | 55 | 391.55 | (M-acetyl) |
| | 3-yl)-N-(1,1,3,3-tetramethyl-butyl)-acetamide | | | 350.4 |
| 186 | 5-{3-[Acetyl-(1,1,3,3-tetramethyl-butyl)- | 65 | 411.5 | (M-acetyl) |
| | amino]-7-methyl-imidazo[1,2-a]pyridin-2-yl}- | | | 370.5 |
| | furan-2-carboxylic acid | | | |
| 187 | N-Cyclohexyl-N-(7-methyl-2-pyridin-3-yl- | 67 | 348.44 | (M-acetyl) |
| | imidazo[1,2-a]pyridin-3-yl)-acetamide | | | 307.4 |
| 188 | N-[2-(5-[1,3]Dioxolan-2-yl-furan-2-yl)-5,7- | 50 | 453.58 | (M-acetyl) |
| | dimethyl-imidazo[1,2-a]pyridin-3-yl]-N- | | | 412.3 |
| | (1,1,3,3-tetramethyl-butyl-acetamide) | | | |
| 189 | N-(2-Benzofuran-2-yl-5,7-dimethyl- | 63 | 423.51 | (M-acetyl) |
| | imidazo(1,2-a]pyridin-3-yl)-N-(2,6-dimethyl- | | | 382.5 |
| | phenyl)-acetamide | | | |
| 190 | N-Tert-butyl-N-(2-furan-2-yl-7-methyl- | 69 | 311.38 | (M-acetyl) |
| | imidazo[1,2-a]pyridin-3-yl)-acetamide | | | 270.3 |
| 191 | N-Tert-butyl-N-(7-methyl-2-pyridin-2-yl- | 57 | 322.41 | (M-acetyl) |
| | imidazo[1,2-a]pyridin-3-yl)-acetamide | | | 281.3 |
| 192 | N-Cyclohexyl-N-(5,7-dimethyl-2-pyridin-3-yl- | 72 | 362.47 | (M-acetyl) |
| | imidazo[1,2-a]pyridin-3-yl)-acetamide | | | 321.4 |
| 193 | N-[2-(2,3-Dichloro-phenyl)-5,7-dimethyl- | 55 | 460.44 | (M-acetyl) |
| | imidazo[1,2-a]pyridin-3-yl]-N-(1,1,3,3- | | | 418.3/420.3/ |
| | tetramethyl-butyl)-acetamide | | | 421.1 |
| 194 | N-[2-(2,3-Dimethoxy-phenyl)-5,7-dimethyl- | 59 | 451.61 | (M-acetyl) |
| | imidazo[1,2-a]pyridin-3-yl]-N-(1,1,3,3- | | | 410.4 |
| | tetramethyl-butyl)-acetamide | | | |
| 195 | N-{2-[3-(4-Chloro-phenoxy)-phenyl]- | 54 | 481.98 | (M-acetyl) |
| | imidazo[1,2-a]pyridin-3-yl}-N-(2,6-dimethyl- | | | 440.4/441.4/ |
| | phenyl)-acetamide | | | 442.4 |
| 196 | N-[2-(5-[1,3]Dioxolan-2-yl-furan-2-yl)-7- | 54 | 439.55 | (M-acetyl) |
| | methyl-imidazo[1,2-a]pyridin-3-yl]-N- | | | 398.4 |
| | (1,1,3,3-tetramethyl-butyl)-acetamide | | | |
| 197 | 5-[3-(Acetyl-cyclohexyl-amino)-5,7-dimethyl- | 70 | 395.45 | (M-acetyl) |
| | imidazo[1,2-a]pyridin-2-yl]-furan-2-carboxylic | | | 354.4 |
| | acid | | | |
| 198 | N-Tert-butyl-N-[7-methyl-2-(5-nitro-furan-2- | 54 | 356.38 | (M-acetyl) |
| | yl)-imidazo[1,2-a]pyridin-3-yl]-acetamide | | | 314.4 |
| 199 | N-[2-(2-Methoxy-phenyl)-7-methyl- | 57 | 407.55 | (M-acetyl) |
| | imidazo[1,2-a]pyridin-3-yl]-N-(1,1,3,3- | | | 366.4 |
| | tetramethyl-butyl)-acetamide | | | |
| 200 | N-[2-(5-Methyl-furan-2-yl)-imidazo[1,2-a]- | 58 | 367.49 | (M-acetyl) |
| | pyridin-3-yl]-N-(1,1,3,3-tetramethyl-butyl)- | | | 326.4/327.4 |
| | acetamide | | | |
| 201 | 5-[3-(Acetyl-tert-butyl-amino)-7-methyl- | 54 | 355.39 | (M-acetyl) |
| | imidazo[1,2-a]pyridin-2-yl]-furan-2-carboxylic | | | 314.4 |
| | acid | | | |
| 202 | N-[2-(4,5-Dimethyl-furan-2-yl)-imidazo[1,2- | 57 | 381.51 | (M-acetyl) |
| | a]pyridin-3-yl]-N-(1,1,3,3-tetramethyl-butyl)- | | | 340.6 |
| | acetamide | | | |
| 203 | N-Cyclohexyl-N-(2-furan-2-yl-5,7-dimethyl- | 60 | 351.44 | (M-acetyl) |
| | imidazo[1,2-a]pyridin-3-yl)-acetamide | | | 310.4 |
| 204 | N-Tert-butyl-N-(7-methyl-2-naphthalen-1-yl- | 54 | 371.48 | (M-acetyl) |
| | imidazo[1,2-a]pyridin-3-yl)-acetamide | | | 330.4 |
| 205 | 5-{3-[Acetyl-(1,1,3,3-tetramethyl-butyl)- | 52 | 414.52 | (M-acetyl) |
| | amino]-imidazo[1,2-a]pyrazin-2-yl]- | | | 373.4 |
| | thiophene-2-carboxylic acid | | | |
| 206 | N-Butyl-N-(2-o-tolyl-imidazo[1,2-a]pyrimidin- | 54 | 322.41 | 339.4/340.4 |
| | 3-yl)-acetamide | | | |
| 207 | N-[2-(3,4-Dimethoxy-phenyl)-7-methyl- | 51 | 429.51 | (M-acetyl) |
| | imidazo[1,2-a]pyridin-3-yl]-N-(2,6-dimethyl- | | | 388.4 |
| | phenyl)-acetamide | | | |
| 208 | N-Tert-butyl-N-(7-methyl-2-phenanthren-9 | 63 | 421.54 | (M-acetyl) |
| | yl-imidazo[1,2-a]pyridin-3-yl)-acetamide | | | 380.5 |
| 209 | N-(2,6-Dimethyl-phenyl)-N-[2-(2-fluoro- | 62 | 387.45 | (M-acetyl) |
| | phenyl)-7-methyl-imidazo[1,2-a]pyridin-3-yl]- | | | 346.4/347.3 |
| | acetamide | | | |
| 210 | N-[2-(2-Methoxy-phenyl)-8-methyl- | 53 | 407.55 | (M-acetyl) |
| | imidaz[1,2-a]pyridin-3-yl]-N-(1,1,3,3- | | | 366.4 |
| | tetramethyl-butyl)-acetamide | | | |
| 211 | N-(2,6-Dimethyl-phenyl)-N-[2-(3-hydroxy- | 73 | 399.49 | (M-acetyl) |
| | phenyl)-5,7-dimethyl-imidazo[1,2-a]pyridin- | | | 358.4 |
| | 3-yl]-acetamide | | | |
| 212 | N-(2-Tert-butyl-7-methyl-imidazo[1,2-a]- | 68 | 349.47 | (M-acetyl) |
| | pyridin-3-yl)-N-(2,6-dimethyl-phenyl)- | | | 308.4 |
| | acetamide | | | |
| 213 | Acetic acid 4-{3-[acetyl-(2,6-dimethyl- | 69 | 536.43 | (M-acetyl) |
| | phenyl)-amino]-6-bromo-8-methyl- | | | 494.3/497.3 |
| | imidazo[1,2-a]pyridin-2-yl}-2-methoxy-phenyl | | | |
| | ester | | | |
| 214 | N-Tert-butyl-N-[2-(5-[1,3]dioxolan-2-yl-furan- | 56 | 383.44 | (M-acetyl) |
| | 2-yl)-7-methyl-imidazo[1,2-a]pyridin-3-yl]- | | | 336.2 |
| | acetamide | | | |
| 215 | [6-(Acetyl-{7-methyl-2-[5-(2-nitro-phenyl)- | 50 | 486.55 | 487.5; (M- |
| | furan-2-yl]-imidazo[1,2-a]pyridin-3-yl}- | | | acetyl) |
| | amino)-hexyl]-methylidyne-ammonium | | | 444.5 |
| 216 | N-(2-Benzofuran-2-yl-7-methyl-imidazo[1,2- | 50 | 417.55 | (M-acetyl) |
| | a]pyridin-3-yl)-N-(1,1,3,3-tetramethyl-butyl)- | | | 376.4/377.3 |
| | acetamide | | | |
| 217 | N-(2-Benzofuran-2-yl-5,7-dimethyl- | 63 | 431.57 | (M-acetyl) |
| | imidazo[1,2-a]pyridin-3-yl)-N-(1,1,3,3- | | | 390.4/391.4 |
| | tetramethyl-butyl)-acetamide | | | |
| 218 | 5-[3-Acetyl-tert-butyl-amino)-imidazo[1,2-a]- | 60 | 357.43 | (M-acetyl) |
| | pyridin-2-yl]-thiophene-2-carboxylic acid | | | 316.5 |
| 219 | N-(2-cyclohexyl-5,7-dimethyl-imidazo[1,2- | 62 | 397.6 | (M-acetyl) |
| | a]pyridin-3-yl)-N-(1,1,3,3-tetramethyl-butyl)- | | | 356.5 |
| | acetamide | | | |
| 220 | N-Tert-butyl-N-[2-(5-methyl-furan-2-yl)- | 61 | 311.38 | (M-acetyl) |
| | imidazo[1,2-a]pyridin-3-yl]-acetamide | | | 270.4 |
| 221 | N-Tert-butyl-N-[2-(5-methylsulfanyl- | 54 | 360.5 | (M-acetyl) |
| | thiophen-2-yl)-imidazo[1,2-a]pyrimidin-3-yl]- | | | 319.4 |
| | acetamide | | | |
| 222 | N-[2-(4,5-Dimethyl-furan-2-yl)-5-methyl- | 53 | 395.54 | (M-acetyl) |
| | imidazo[1,2-a]pyridin-3-yl]-N-(1,1,3,3- | | | 354.5 |
| | tetramethyl-butyl)-acetamide | | | |
| 223 | N-Butyl-N-[2-(2,4-dichloro-phenyl)- | 62 | 377.27 | 377.4/379.4 |
| | imidazo[1,2-a]pyrimidin-3-yl]-acetamide | | | |
| 224 | N-[2-(3-Bromo-thiophen-2-yl)-6-methyl- | 53 | 432.38 | (M-acetyl) |
| | imidazo[1,2-a]pyridin-3-yl]-N-cyclohexyl- | | | 390.4/392.4 |
| | acetamide | | | |
| 225 | 5-{3-[Acetyl-(2,6-dimethyl-phenyl)-amino]-6- | 55 | 419.5 | (M-acetyl) |
| | methyl-imidazo[1,2-a]pyridin-2-yl}- | | | 378.4 |
| | thiophene-2-carboxylic acid | | | |
| 226 | N-Butyl-N-[2-(2,3-dimethoxy-phenyl)-7- | 62 | 381.47 | 382.5 |
| | methyl-imidazo[1,2-a]pyridin-3-yl]-acetamide | | | |
| 227 | N-Tert-butyl-N-[2-(2,3-dichloro-phenyl)- | 54 | 376.28 | (M-acetyl) |
| | imidazo[1,2-a]pyridin-3-yl]-acetamide | | | 334.3/336.3 |
| 228 | N-(2-Furan-2-yl-5-propyl-imidazo[1,2-a]- | 53 | 395.54 | (M-acetyl) |
| | pyridin-3-yl)-N-(1,1,3,3-tetramethyl-butyl)- | | | 354.4 |
| | acetamide | | | |
| 229 | 5-[3-(Acetyl-cyclohexyl-amino)-imidazo[1,2- | 59 | 383.46 | (M-acetyl) |
| | a]pyridin-2-yl]-thiophene-2-carboxylic acid | | | 342.5 |
| 230 | 5-{3-[Acetyl-(1,1,3,3-tetramethyl-butyl)- | 52 | 411.5 | (M-acetyl) |
| | amino]-8-methyl-imidazo[1,2-a]pyridin-2-yl}- | | | 370.6 |
| | furan-2-carboxylic acid | | | |
| 231 | 3-(Acetyl-butyl-amino)-2-pyridin-2-yl- | 52 | 352.39 | 353.5 |
| | imidazo[1,2-a]pyridine-8-carboxylic acid | | | |
| 232 | {6-[Acetyl-(5,7-dimethyl-2-pyridin-3-yl- | 59 | 390.51 | (M-acetyl) |
| | imidazo[1,2-a]pyridin-3-yl)-amino]-hexyl}- | | | 348.5 |
| | methylidyne-ammonium | | | |
| 233 | N-Tert-butyl-N-[2-(5-methylsulfanyl- | 53 | 360.5 | (M-acetyl) |
| | thiophen-2-yl)-imidazo[1,2-a]pyrazin-3-yl]- | | | 319.2 |
| | acetamide | | | |
| 234 | 5-[3-(Acetyl-cyclohexyl-amino)-5-methyl- | 58 | 397.49 | (M-acetyl) |
| | imidazo[1,2-a]pyridin-2-yl]-thiophene-2- | | | 356.4 |
| | carboxylic acid | | | |
| 235 | N-[2-(5-Methylsulfanyl-thiophen-2-yl)- | 58 | 416.6 | (M-acetyl) |
| | imidazo[1,2-a]pyrazin-3-yl]-N-(1,1,3,3- | | | 375.3 |
| | tetramethyl-butyl)-acetamide | | | |
| 236 | N-[2-(2,3-Dichloro-phenyl)-5-methyl- | 58 | 438.35 | (M-acetyl) |
| | imidazo[1,2-a]pyridin-3-yl]-N-(2,6-dimethyl- | | | 396.3/398.3 |
| | phenyl)-acetamide | | | |
| 237 | N-Butyl-N-[2-(2-methoxy-phenyl)- | 55 | 337.42 | 338.5 |
| | imidazo[1,2-a]pyridin-3-yl]-acetamide | | | |
| 238 | (6-{Acetyl-[2-(2-methoxy-phenyl)-6-nitro- | 52 | 436.49 | 436.5 |
| | imidazo[1,2-a]pyridin-3-yl]-amino}-hexyl)- | | | |
| | methylidyne-ammonium | | | |
| 239 | N-(2-Benzofuran-2-yl-7-methyl-imidazo[1,2- | 52 | 409.48 | (M-acetyl) |
| | a]pyridin-3-yl)-N-(2,6-dimethyl-phenyl- | | | 368.5 |
| | acetamide | | | |
| 240 | (6-{Acetyl-[2-(2-methoxy-phenyl)-5,7- | 61 | 419.54 | (M-acetyl) |
| | dimethyl-imidazo[1,2-a]pyridin-3-yl]-amino}- | | | 377.5 |
| | hexyl)-methylidyne-ammonium | | | |
| 241 | {6-[Acetyl-(7-methyl-2-pyridin-3-yl- | 62 | 376.48 | (M-acetyl) |
| | imidazo[1,2-a]pyridin-3-yl)-amino]-hexyl}- | | | 334.5 |
| | methylidyne-ammoniunl | | | |
| 242 | N-(6-Methyl-2-p-tolyl-imidazo[1,2-a]pyridin- | 58 | 391.55 | (M-acetyl) |
| | 3-yl)-N-(1,1,3,3-tetramethyl-butyl)-acetamide | | | 350.4 |
| 243 | Acetic acid 5-{3-[acetyl-(2,6-dimethyl- | 62 | 445.51 | (M-acetyl) |
| | phenyl)-amino]-5,7-dimethyl-imidazo[1,2-a]- | | | 404.4 |
| | pyridin-2-yl}-furan-2-yl methyl ester | | | |
| 244 | {Acetyl-[2-(3-hydroxy-phenyl)-6-methyl- | 50 | 353.37 | 354.4; (M- |
| | imidazo[1,2-a]pyridin-3-yl]-amino}-acetic | | | acetyl) |
| | acid methyl ester | | | 312.4 |
| 245 | N-Tert-butyl-N-[2-(2-trifluorO-methyl- | 56 | 375.39 | (M-acetyl) |
| | phenyl)-imidazo[1,2-a]pyridin-3-yl]- | | | 334.3 |
| | acetamide | | | |
| 246 | N-Butyl-N-[2-(2-chloro-4-nuoro-phenyl)- | 55 | 359.83 | 360.4 |
| | imidazo[1,2-a]pyridin-3-yl]-acetamide | | | |
| 247 | N-[2-(2,4-Dichloro-phenyl)-imidazo[1,2-a]- | 56 | 424.33 | (M-acetyl) |
| | pyridin-3-yl]-N-(2,6-dimethyl-phenyl)- | | | 382.4/384.3 |
| | acetamide | | | |
| 248 | 5-{3-[Acetyl-(1,1,3,3-tetramethyl-butyl)- | 52 | 412.48 | (M-acetyl) |
| | amino]-7-methyl-imidazo[1,2-a]pyrimidin-2- | | | 371.8 |
| | yl}-furan-2-carboxylic acid | | | |
| 249 | Acetic acid 5-{3-[acetyl-(1,1,3,3-tetramethyl- | 54 | 426.51 | (M-acetyl) |
| | butyl)-amino]-imidazo[1,2-a]pyrimidin-2-yl}- | | | 385.4 |
| | furan-2-yl methyl ester | | | |
| 250 | N-(2,7-Dimethyl-imidazo[1,2-a]pyridin-3-yl)- | 58 | 315.46 | (M-acetyl) |
| | N-(1,1,3,3-tetramethyl-butyl)-acetamide | | | 274.5 |
| 251 | Acetic acid 4-[3-(acetyl-cyclohexyl-amino)-5- | 51 | 471.94 | 472.4; |
| | amino-7-chloro-imidazo[1,2-a]pyrimidin-2- | | | (M-acetyl) |
| | yl]-2-methoxy-phenyl ester | | | 430.4/432.4 |
| 252 | Acetic acid 4-(3-(acetyl-cyclohexyl-amino)- | 53 | 450.53 | (M-acetyl) |
| | 5,7-dimethyl-imidazo[1,2-a]pyrimidin-2-yl]-2- | | | 409.5 |
| | methoxy-phenyl ester | | | |
| 253 | N-[6-Bromo-2-(2-chloro-6-fluoro-phenyl)-8- | 51 | 478.79 | (M-acetyl) |
| | methyl-imidazo[1,2-a]pyridin-3-yl]-N- | | | 436.4/438.3/ |
| | cyclohexyl-acetamide | | | 440.3 |
| 254 | N-[2-(2-chloro-6-fluoro-phenyl)-5-methyl- | 51 | 399.89 | (M-acetyl) |
| | imidazo[1,2-a]pyridin-3-yl]-N-cyclohexyl- | | | 358.3 |
| | acetamide | | | |
| 255 | N-Butyl-N-[2-(2,3-dichloro-phenyl)- | 65 | 377.27 | 377.4/379.4 |
| | imidazo[1,2-a]pyrimidin-3-yl]-acetamide | | | |
| 256 | N-[2-(5-chloro-thiophen-2-yl)-imidazo[1,2-a]- | 55 | 404.96 | (M-acetyl) |
| | pyrimidin-3-yl]-N-(1,1,3,3-tetramethyl-butyl)- | | | 363.3/365.3/ |
| | acetamide | | | 367.3 |
| 257 | [Acetyl-(2-cyclohexyl-7-methyl-imidazo[1,2- | 56 | 343.42 | (M-acetyl) |
| | a]pyridin-3-yl)-amino]-acetic acid methyl | | | 302.5 |
| | ester | | | |
| 258 | N-Tert-butyl-N-[2-(2-chloro-6-fluoro-phenyl)- | 60 | 359.83 | (M-acetyl) |
| | imidazo[1,2-a]pyridin-3-yl]-acetamide | | | 318.3/320.3 |
| 259 | N-Cyclohexyl-N-(5,7-dimethyl-2-pyridin-2-yl- | 73 | 362.47 | (M-acetyl) |
| | imidazo[1,2-a]pyridin-3-yl)-acetamide | | | 321.4 |
| 260 | Acetic acid 5-[3-(acetyl-cyclohexyl-amino)-5- | 51 | 409.48 | (M-acetyl) |
| | methyl-imidazo[1,2-a]pyridin-2-yl]-furan-2-yl | | | 368.6 |
| | methyl ester | | | |
| 261 | N-(2,6-Dimethyl-phenyl)-N-[6-methyl-2-(2- | 61 | 437.46 | (M-acetyl) |
| | trfluorO-methyl-phenyl)-imidazo[1,2-a]- | | | 396.4 |
| | pyridin-3-yl]-acetamide | | | |
| 262 | N-Cyclohexyl-N-(2-furan-2-yl-7-methyl- | 54 | 337.42 | (M-acetyl) |
| | imidazo[1,2-a]pyridin-3-yl)-acetamide | | | 296.5 |
| 263 | N-Cyclohexyl-N-(7-methyl-2-pyridin-2-yl- | 64 | 348.44 | 349.4; (M- |
| | imidazo[1,2-a]pyridin-3-yl)-acetamide | | | acetyl) |
| | | | | 307.4 |
| 264 | N-Cyclohexyl-N-[2-(5-[1,3]dioxolan-2-yl- | 50 | 409.48 | (M-acetyl) |
| | furan-2-yl)-5-methyl-imidazo[1,2-a]pyridin-3- | | | 368.4 |
| | yl]-acetamide | | | |
| 265 | N-Tert-butyl-N-(5-propyl-2-pyridin-3-yl- | 54 | 350.46 | (M-acetyl) |
| | imidazo[1,2-a]pyridin-3-yl)-acetamide | | | 309.3 |
| 266 | N-Tert-butyl-N-[2-(5-methyl-thiophen-2-yl)- | 53 | 328.43 | (M-acetyl) |
| | imidazo[1,2-a]pyrimidin-3-yl]-acetamide | | | 287.3 |
| 267 | 3-[Acetyl-(1,1,3,3-tetramethyl-butyl)-amino]- | 62 | 397.47 | (M-acetyl) |
| | 2-furan-2-yl-imidazo[1,2-a]pyridine-8- | | | 356.7 |
| | carboxylic acid | | | |
| 268 | N-Tert-butyl-N-[2-(4,5-dimethyl-furan-2-yl)-6- | 51 | 339.43 | (M-acetyl) |
| | methyl-imidazo[1,2-a]pyridin-3-yl]-acetamide | | | 298.4 |
| 269 | N-{2-[3-(4-Chloro-phenoxy)-phenyl]- | 51 | 459.97 | 460.4/462.4; |
| | imidazo[1,2-a]pyridin-3-yl}-N-cyclohexyl- | | | (M-acetyl) |
| | acetamide | | | 418.5/419.4 |
| 270 | Acetic acid 4-[3-(acetyl-cyclohexyl-amino)- | 52 | 422.48 | 423.4; (M- |
| | imidazo[1,2-a]pyrimidin-2-yl]-2-methoxy- | | | acetyl) 381.4 |
| | phenyl ester | | | |
| 271 | N-[2-(5-Bromo-furan-2-yl)-8-methyl- | 51 | 446.39 | (M-acetyl) |
| | imidazo[1,2-a]pyridin-3-yl]-N-(1,1,3,3- | | | 404.4/406.3 |
| | tetramethyl-butyl)-acetamide | | | |
| 272 | N-(2,6-Dimethyl-phenyl)-N-[2-(3-hydroxy- | 54 | 400.48 | 359.5 (M- |
| | phenyl)-5,7-dimethyl-imidazo[1,2-a]- | | | acetyl) 401.4 |
| | pyrimidin-3-yl]-acetamide | | | |
| 273 | N-Cyclohexyl-N-[2-(2,3-dichloro-phenyl)- | 55 | 402.32 | (M-acetyl) |
| | imidazo[1,2-a]pyridin-3-yl]-acetamide | | | 360.4/362.4 |
| 274 | N-Cyclohexyl-N-[2-(2,4-dichloro-phenyl)-5- | 64 | 416.35 | (M-acetyl) |
| | methyl-imidazo[1,2-a]pyridin-3-yl]-acetamide | | | 374.4/376.3 |
| 275 | N-Cyclohexyl-N-[2-(2,4-dichloro-phenyl)- | 59 | 403.31 | (M-acetyl) |
| | imidazo[1,2-a]pyrimidin-3-yl]-acetamide | | | 361.4/363.3 |
| 276 | [Acetyl-(2-o-tolyl-imidazo[1,2-a]pyrazin-3-yl)- | 50 | 338.36 | (M-acetyl) |
| | amino]-acetic acid methyl ester | | | 297.4 |
| 277 | N-Tert-butyl-N-(6,8-dichloro-2-thiophen-2-yl- | 53 | 382.31 | (M-acetyl) |
| | imidazo[1,2-a]pyridin-3-yl)-acetamide | | | 340.3/342.2 |
| 278 | N-Tert-butyl-N-(5-propyl-2-thiophen-2-yl- | 55 | 355.5 | (M-acetyl) |
| | imidazo[1,2-a]pyridin-3-yl)-acetamide | | | 340.3/342.2 |
| 279 | {6-[Acetyl-(7-methyl-2-p-tolyl-imidazo[1,2-a]- | 71 | 389.52 | 389.6 (M |
| | pyridin-3-yl)-amino]-hexyl}-methylidyne- | | | acetyl) 347.6 |
| | ammonium | | | |
| 280 | N-Butyl-N-(6-methyl-2-p-tolyl-imidazo[1,2-a]- | 53 | 335.45 | 336.5; (M- |
| | pyridin-3-yl)-acetamide | | | acetyl) 294.5 |
| 281 | (6-{Acetyl-[2-(2-methoxy-phenyl)-7-methyl- | 62 | 405.52 | 405.5 |
| | imidazo[1,2-a]pyridin-3-yl]-amino}-hexyl)- | | | |
| | methylidyne-ammonium | | | |
| 282 | 5-{3-[Acetyl-(1,1,3,3-tetramethyl-butyl)- | 51 | 411.5 | (M-acetyl) |
| | amino]-6-methyl-imidazo[1,2-a]pyridin-2-yl}- | | | 370.4 |
| | furan-2-carboxylic acid | | | |
| 283 | N-Butyl-N-[2-(3,4,5-trimethoxy-phenyl)- | 51 | 398.46 | 399.5; (M- |
| | imidazo[1,2-a]pyrazin-3-yl]-acetamide | | | acetyl) 357.5 |
| 284 | N-Butyl-N-[2-(3-hydroxy-phenyl)- | 51 | 324.38 | 325.4; (M- |
| | imidazo[1,2-a]pyrimidin-3-yl]-acetamide | | | acetyl) 283.3 |
| 285 | [Acetyl-(2-o-tolyl-imidazo[1,2-a]pyrimidin-3- | 51 | 338.36 | 339.3; (M- |
| | yl)-amino]-acetic acid methyl ester | | | acetyl) 297.4 |
| 286 | N-(2-Benzofuran-2-yl-8-methyl-imidazo[1,2- | 59 | 417.55 | (M-acetyl) |
| | a]pyridin-3-yl)-N-(1,1,3,3-tetramethyl-butyl)- | | | 376.4/377.4 |
| | acetamide | | | |
| 287 | N-Butyl-N-(7-methyl-2-p-tolyl-imidazo[1,2-a]- | 58 | 336.43 | 337.5 |
| | pyrimidin-3-yl)-acetamide | | | |
| 288 | N-Tert-butyl-N-(6,8-dibromo-2-methyl- | 62 | 403.12 | (M-acetyl) |
| | imidazo[1,2-a]pyridin-3-yl)-acetamide | | | 362.2 |
| 289 | {6-[Acetyl-(5,7-dimethyl-2-pyridin-2-yl- | 63 | 390.51 | (M-acetyl) |
| | imidazo[1,2-a]pyridin-3-yl)-amino]-hexyl}- | | | 348.5 |
| | methylidyne-ammonium | | | |
| 290 | N-Tert-butyl-N-[2-(2-ethoxy-naphthalen-1- | 67 | 415.53 | (M-acetyl) |
| | yl)-7-methyl-imidazo[1,2-a]pyridin-3-yl]- | | | 374.4 |
| | acetamide | | | |
| 291 | N-Tert-butyl-N-[2-(2-chloro-4-fluoro-phenyl)- | 53 | 359.83 | (M-acetyl) |
| | imidazo[1,2-a]pyridin-3-yl]-acetamide | | | 318.3/319.2 |

General Working Procedure 3 (GWP 3)

(Equivalents Mean Substance Equivalents Relative to the Isonitrile Used):

1.15 equivalents of the heterocyclic amine having the general formula II were first suspended or dissolved in dichloromethane (2 ml per mmol of isonitrile IV used) in a reaction vessel. 1.5 equivalents of aldehyde III, one equivalent of isonitrile IV and finally aqueous perchloric acid solution (20 m %; 0.098 ml per mmol of isonitrile used) were added thereto in succession, and the batch was stirred for 20 hours at room temperature.

For working up, saturated sodium chloride solution (about 5 ml per mmol of isonitrile used) and dichloromethane (about 4 ml per mmol of isonitrile used) were added, the phases were separated, and the organic phase was then extracted twice using dichloromethane (about 2 ml per mmol of isonitrile used). The combined organic phases were washed in succession with buffer solution (pH 10; Merck art. no. 1.09438.1000; about 2 ml per mmol of isonitrile used) and sat. sodium chloride solution (about 2 ml per mmol of isonitrile used), dried over sodium sulfate, filtered, concentrated in vacuo using a rotary evaporator and freed of solvent residues under an oil-pump vacuum.

The resulting crude product was either conveyed directly to a hydrochloride precipitation (dissolution of the crude base in about 10 ml of 2-butanone per gram of base; addition of half a molar equivalent of water, followed by 1.1 molar equivalents of chlorotrimethylsilane and stirring overnight), or heated to reflux with hexane (about 10 ml per mmol of isonitrile used), with stirring. If the product did not dissolve completely, it was separated off in the hot state. After cooling of the hexane solution, any solid obtained was filtered off and dried under an oil-pump vacuum. Any precipitations were treated analogously separately. The resulting filtrate was concentrated using a rotary evaporator and the residue was again dried under an oil-pump vacuum. Up to four fractions were obtained in that manner:

0: no treatment with hexane

1: residue insoluble in hexane

2: solid precipitated from hexane solution upon cooling

3: post-precipitation

4: residue from hexane solution concentrated to dryness

From the fractions obtained in each particular case, the product fraction(s) (generally the solid precipitated from the hexane solution) was/were identified by thin-layer chromatographic and/or NMR spectroscopic investigations.

Finally, a hydrochloride was precipitated from a portion of a product fraction (see above).

Examples 292-298 prepared according to GWP 3 were tested in the citrulline assay; the results are shown in Table 4. The following were also prepared by way of example according to GWP 3: cyclohexyl-(7-methyl-2-phenyl-imidazo[1,2-a]pyridin-3-yl)-amine hydrochloride, (2-furan-2-yl-7-methyl-imidazo[1,2-a]pyrimidin-3-yl)-(1,1,3,3-tetramethyl-butyl)-amine hydrochloride and tert-butyl-[2-(4-nitro-phenyl)-imidazo[1,2-a]pyrazin-3-yl]-amine hydrochloride.

TABLE 4

| | | | | | Citrulline |
|---|---|---|---|---|---|
| | | Batch | | | assay |
| Example | | mmol | Yield | Product | IC50 |
| No. | Name | isonitrile | g product fraction | fraction | (μM) |
| 292 | Cyclohexyl-[7-methyl-2-(2-trifluorO-methyl- | 21.3 | 5.93 | 0 | 2.4 |
| | phenyl)-imidazo[1,2-a]pyridin-3-yl]-amine; | | | | |
| | hydrochloride | | | | |
| 293 | Tert-butyl-(2-furan-2-yl-5,7-dimethyl- | 18.8 | 4.64 | 0 | 2.8 |
| | imidazo[1,2-a]pyridin-3-yl)-amine; | | | | |
| | hydrochloride | | | | |
| 294 | Tert-butyl-(7-methyl-2-phenyl-imidazo[1,2- | 54.1 | 9.06 | 2 | 2.4 |
| | a]pyridin-3-yl)-amine; hydrochloride | | | | |
| 295 | Cyclohexyl-(5,7-dimethyl-2-pyridin-4-yl- | 50.4 | 12.2 | 2 | 9.2 |
| | imidazo[1,2-a]pyridin-3-yl)-amine; | | | | |
| | hydrochloride | | | | |
| 296 | (2-Furan-2-yl-5,7-dimethyl-imidazo[1,2-a]- | 47.9 | 13.9 | 4 | 2.5 |
| | pyridin-3-yl)-(1,1,3,3-tetramethyl-butyl)- | | | | |
| | amine; hydrochloride | | | | |
| 297 | Tert-butyl-(2,5,7-trimethyl-imidazo[1,2-a]- | 48.1 | 10.5 | 2 + 4 | 9.2 |
| | pyridin-3-yl)-amine; hydrochloride | | | | |
| 298 | [2-(2-Fluorophenyl)-7-methyl-imidazo[1,2- | 43.1 | 15.9 | 4 | 4.3 |
| | a]pyridin-3-yl]-(1,1,3,3-tetramethyl-butyl)- | | | | |
| | amine; hydrochloride | | | | |
| 299 | Cyclohexyl-(7-methyl-2-phenyl-imidazo[1,2- | 5.00 | 1.64 | 2 | |
| | a]pyridin-3-yl)-amine; hydrochloride | | | | |
| 300 | (2-Furan-2-yl-7-methyl-imidazo[1,2-a]- | 43.1 | 10.2 | 2 + 3 | |
| | pyrimidin-3-yl)-(1,1,3,3-tetramethyl-butyl)- | | | | |
| | amine; hydrochloride | | | | |
| 301 | Tert-butyl-(2-(4-nitro-phenyl)-imidazo[1,2- | 48.1 | 15.1 | 2 | |
| | a]pyrazin-3-yl]-amine; hydrochloride | | | | |

As comparison example, the NOS inhibitor 7-nitroindazole known from the prior art, having an IC₅₀ value of 5.23 μM, was tested in the citrulline assay.

The compounds of Examples 302 to 312 were also prepared according to GWP 3 and d in the above-described NOS assay for % inhibition. The results are shown in Table 5.

TABLE 5

| | | | Batch | Yield | |
|---|---|---|---|---|---|
| Example | | NOS assay | mmol | g product | Product |
| No. | Name | % inhibition | isonitrile | fraction | fraction |
| 302 | Cyclohexyl-(2-furan-3-yl-5,7-dimethyl-imidazo- | 54 | 9.2 | 1.9 | 2 |
| | [1,2-a]pyridin-3-yl)-amine hydrochloride | | | | |
| 303 | Cyclopentyl-(2-furan-3-yl-5,7-dimethyl- | 57 | 10.5 | 2.1 | 2 |
| | imidazo[1,2-a]pyridin-3-yl)-amine | | | | |
| | hydrochloride | | | | |
| 304 | [2-(4-Bromo-2-fluoro-phenyl)-5,7-dimethyl- | 56 | 6.3 | 1.9 | 4 |
| | imidazo[1,2-a]pyridin-3-yl]-cyclopentyl-amine | | | | |
| | hydrochloride | | | | |
| 305 | Cyclopentyl-{5,7-dimethyl-2-[5-(2-nitro-phenyl)- | 55 | 6.3 | 3 | 2 |
| | furan-2-yl]-imidazo[1,2-a]pyridin-3-yl}-amine | | | | |
| | hydrochloride | | | | |
| 306 | {2-[5-(4-Chlorophenyl)-furan-2-yl]-5,7-dimethyl- | 73 | 6.7 | 1.7 | 2 |
| | imidazo[1,2-a]pyridin-3-yl}-cyclopentyl-amine | | | | |
| | hydrochloride | | | | |
| 307 | Cyclopentyl-(2-furan-3-yl-5,7-dimethyl- | 57 | 10.5 | 2.6 | 4 |
| | imidazo(1,2-a]pyridin-3-yl)-amine | | | | |
| | hydrochloride | | | | |
| 308 | (2-Furan-3-yl-5,7-dimethyl-imidazo[1,2-a]- | 50 | 25.1 | 5.6 | 2 |
| | pyridin-3-yl)-(1,1,3,3-tetramethyl-butyl)-amine | | | | |
| | hydrochloride | | | | |
| 309 | Benzyl-(7-methyl-2-thiophen-3-yl-imidazo- | 45 | 21.3 | 4.1 | 2 |
| | [1,2-a]pyridin-3-yl)-amine hydrochloride | | | | |
| 310 | Cyclohexyl-(2-furan-3-yl-5,7-dimethyl-imidazo- | 52 | 22.9 | 5.2 | 2 |
| | [1,2-a]pyridin-3-yl)-amine hydrochloride | | | | |
| 311 | (2-Furan-3-yl-7-methyl-imidazo[1,2-a]pyridin-3- | 45 | 25.1 | 2.9 | 2 |
| | yl)-(1,1,3,3-tetramethyl-butyl)-amine | | | | |
| | hydrochloride | | | | |
| 312 | (5,7-Dimethyl-2-thiophen-3-yl-imidazo[1,2-a]- | 74 | 18 | 4.6 | 4 |
| | pyridin-3-yl)-(1,1,3,3-tetramethyl-butyl)-amine | | | | |
| | hydrochloride | | | | |

General Working Procedure 4 (GWP 4):

The starting material (product fraction) obtained according to GWP 3 was placed in a reaction vessel in tetrahydrofuran (about 3 ml per mmol of starting material); 1.10 substance equivalents of n-butyllithium solution in hexane (1.6 mol/l) were added dropwise, with stirring, at from −15 to −5° C., and stirring was continued for one hour. 1.05 substance equivalents of the acetyl chloride were then added dropwise, and stirring was carried out overnight with heating at room temperature.

For working up, cooling to 0 to 5° C. was carried out, and semi-saturated ammonium chloride solution (about 1.5 ml per mmol of starting material) was added. Extraction was carried out three times with ether (about 1.5 ml per mmol of starting material), and the combined extracts were dried over sodium sulfate, filtered and concentrated.

After thin-layer chromatographic and/or NMR spectroscopic investigation, a portion of the product so obtained was conveyed to a hydrochloride precipitation according to GWP 3.

The compounds prepared by way of example according to GWP 4 are N-{2-[3-(4-chlorophenoxy)-phenyl]-imidazo[1,2-a]pyridin-3-yl}-N-cyclohexyl-acetamide hydrochloride, N-cyclohexyl-N-(7-methyl-2-o-tolyl-imidazo[1,2-a]pyridin-3-yl)-acetamide hydrochloride and N-(2,6-dimethyl-phenyl)-N-[2-(2,4-dimethyl-phenyl)-5-methyl-imidazo[1,2-a]pyridin-3-yl]-acetamide hydrochloride.

General Working Procedure 5 (GWP 5):

The starting material obtained according to GWP 4 was placed in a reaction vessel; ten substance equivalents of the respective acid halide were added, with stirring, and stirring was carried out for one hour at 40° C.

The reaction mixture was taken up in a small amount of dichloromethane, and the product was precipitated by addition of ether and, optionally, hexane, and was then recrystallized.

Owing to the water content of the solvents used, the desired product was generally obtained by this procedure in the form of the hydrohalide or, alternatively, was conveyed to a hydrochloride precipitation according to GWP 3.

1-Acetyl-3-(acetyl-cyclohexyl-amino)-7-methyl-2-o-tolyl-imidazo[1,2-a]pyridin-1-ium) chloride hydrochloride was prepared by way of example according to GWP 5.

Pharmaceutical Formulation for the use According to the Invention

1 g of the hydrochloride of (5,7-dimethyl-2-p-tolyl-imidazo[1,2-a]pyridin-3-yl)-(1,1,3,3-tetramethyl-butyl)-amine was dissolved at room temperature in 1 litre of water for injection purposes and then adjusted to isotonic conditions by addition of sodium chloride.

An isotonic solution of 1 g of (5,7-dimethyl-2-thiophen-3-yl-imidazo[1,2-a]pyridin-3-yl)-(1,1,3,3-tetramethyl-butyl)-amine hydrochloride in 1 litre of water was prepared in the same manner.

What is claimed is:

1. A pharmaceutical composition for treating inflammatory pain or migraine, comprising an effective amount of a compound of formula I, or a pharmaceutically acceptable salt thereof, and a pharmaceutically acceptable excipient, 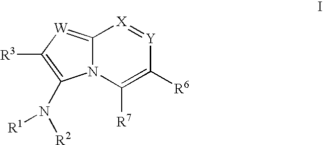
wherein in formula I,
X represents CR⁴ or N,
Y represents CR⁵ or N and
X and Y do not simultaneously represent N,
W represents N or NR⁸,
R¹ represents C₁₋₁₂-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; heterocyclyl, which is saturated or unsaturated, and unsubstituted or mono- or poly-substituted; aryl, which is unsubstituted or mono- or poly-substituted;
heteroaryl, which is unsubstituted or mono- or poly-substituted; C₁₋₈-alkyl-aryl or C₁₋₈-alkyl-heteroaryl, wherein the alkyl is straight-chained or branched, saturated or unsaturated, and unsubstituted or mono- or poly-substituted, the aryl is unsubstituted or mono- or poly-substituted, and the heteroaryl is unsubstituted or mono- or poly-substituted,
R² represents hydrogen or C(═O)R⁹,
R³ represents C₁₋₈-alkyl, which is straight-chained or branched, saturated or unsaturated, and unsubstituted or mono- or poly-substituted; C₃₋₈-cycloalkyl, wherein the cycloalkyl is saturated or unsaturated, and unsubstituted or mono- or poly-substituted; heterocyclyl, which is saturated or unsaturated, and unsubstituted or mono- or poly-substituted; aryl, which is unsubstituted or mono- or poly-substituted; heteroaryl, which is unsubstituted or mono- or poly-substituted; C₁₋₈-alkyl-C₃₋₈-cycloalkyl, C₁₋₈-alkyl-heterocyclyl, C₁₋₈-alkyl-aryl or C₁₋₈-alkyl-heteroaryl, wherein the alkyl is straight-chained or branched, saturated or unsaturated, and unsubstituted or mono- or poly-substituted, the cycloalkyl is saturated or unsaturated and unsubstituted or mono- or poly-substituted, the heterocyclyl is saturated or unsaturated and unsubstituted or mono- or poly-substituted, the aryl is unsubstituted or mono- or poly-substituted, and the heteroaryl is unsubstituted or mono- or poly-substituted,
R⁴, R⁵, R⁶ and R⁷ each independently of the others represents hydrogen, or C₁₋₈-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; F, Cl, Br, I, CN, NO₂, NH₂, C(═O)R⁹, CO₂H, CO₂R¹⁰, OH or OR¹¹, or
R⁴ and R⁵ or R⁵ and R⁶ or R⁶ and R⁷ represent a four-membered saturated or unsaturated hydrocarbon bridge having zero, 1, 2 or 3 hetero atoms selected from the group consisting of N, O and S, and the others of R⁴, R⁵, R⁶ and R⁷ represent hydrogen,
R⁸ represents C(═O)R⁹,
R⁹ represents C₁₋₈-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; heterocyclyl, which is saturated or unsaturated, and unsubstituted or mono- or poly-substituted; aryl, which is unsubstituted or mono- or poly-substituted; heteroaryl, which is unsubstituted or mono- or poly-substituted; C₁₋₈-alkyl-aryl or C₁₋₈-alkyl-heteroaryl, wherein the alkyl is straight-chained or branched, saturated or unsaturated, and unsubstituted or mono- or poly-substituted, the aryl is unsubstituted or mono- or poly-substituted, and the heteroaryl is unsubstituted or mono- or poly-substituted, and
R¹⁰ and R¹¹ each independently of the other represents C₁₋₈-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; aryl, which is unsubstituted or mono- or poly-substituted; C₁₋₈-alkyl-aryl, wherein the alkyl is straight-chained or branched, saturated or unsaturated, and unsubstituted or mono- or poly-substituted, and the aryl is unsubstituted or mono- or poly-substituted.

2. A phamarceutical according to claim 1, wherein in formula I
R¹ represents methyl, ethyl, n-propyl, 2-propyl, n-butyl, tert-butyl, n-hexyl, n-octyl, 1,1,3,3-tetramethylbutyl; CH₂-aryl, which is unsubstituted or mono- or poly-substituted; CH₂CO₂—C₁₋₆-alkyl, wherein the alkyl is straight-chained or branched; CH₂PO(O—C₁₋₆-alkyl)₂, wherein the alkyl is straight-chained or branched; CH₂SiR²R¹³R¹⁴, CH₂CH₂-morpholin-4-yl; (CH₂)ₙ—NC, wherein n=2, 3, 4, 5 or 6; C₃₋₈-cycloalkyl, wherein the cycloalkyl is unsubstituted or mono- or poly-substituted; or phenyl, wherein the phenyl is unsubstituted or mono- or poly-substituted,
R² represents H or C(═O)—C₁₋₄-alkyl,
R³ represents methyl, ethyl, n-propyl, 2-propyl, n-butyl, tert-butyl, cyclopropyl, cyclopentyl or cyclohexyl, which are unsubstituted or mono- or poly-substituted; phenyl, which is unsubstituted or mono- or poly-substituted; 1-naphthyl or 2-naphthyl, wherein the naphthyl is unsubstituted or mono- or poly-substituted; 9-phenanthrenyl, pyrrol-2-yl, pyrrol-3-yl, pyridin-2-yl, pyridin-3-yl or pyridin-4-yl, wherein the pyrrolyl or pyridinyl are unsubstituted or mono- or poly-substituted; furan-2-yl or furan-3-yl, wherein the furanyl is unsubstituted or mono- or poly-substituted; thien-2-yl or thien-3-yl, wherein the thienyl is unsubstituted or mono- or poly-substituted; imidazol-2-yl, imidazol-4-yl, imidazol-5-yl, wherein the imidazolyl is unsubstituted or mono- or poly-substituted; thiazol-2-yl, thiazol-4-yl, thiazol-5-yl, wherein the thiazolyl is unsubstituted or mono- or poly-substituted; oxazol-2-yl, oxazol-4-yl, oxazol-5-yl, wherein the oxazolyl is unsubstituted or mono- or poly-substituted; isooxazol-3-yl, isooxazol-4-yl, isooxazol-5-yl, wherein the isooxazolyl is unsubstituted or mono- or poly-substituted; indol-2-yl, benzofuran-2-yl or benzofuran-3-yl,
R⁴, R⁵, R⁶ and R⁷ each independently of the others represents H, methyl, ethyl, n-propyl, 2-propyl, n-butyl, tert-butyl, CF₃, F, Cl, Br, I, CO₂H, CO₂-methyl, CO₂-ethyl, C(═O)CH₃ or NO₂, or R⁶ and R⁷ form the hydrocarbon bridge —CH═CH—CH═CH—,
R⁸ represents C(═O)CH₃ and
R¹², R¹³ and R¹⁴ each independently of the others represents C₁₋₆-alkyl, which is straight-chained or branched and is unsubstituted or mono- or poly-substituted; C₃₋₈-cycloalkyl or CH₂—C₃₋₈-cycloalkyl, wherein cycloalkyl is unsubstituted or mono- or poly-substituted; or phenyl, wherein phenyl is unsubstituted or mono- or poly-substituted.

3. A pharmaceutical composition according to claim 2, wherein in formula I
R¹ represents methyl, ethyl, n-propyl, 2-propyl, n-butyl, tert-butyl, n-hexyl, n-octyl, 1,1,3,3-tetramethylbutyl; CH₂-aryl, wherein the aryl is unsubstituted or mono- or poly-substituted; CH₂CO₂—C₁₋₆-alkyl, wherein the alkyl is straight-chained or branched; CH₂PO(O—C₁₋₆-alkyl)₂, wherein the alkyl is straight-chained or branched, CH₂SiR²R¹³R⁴, CH₂CH₂-morpholin-4-yl; (CH₂)ₙ—NC, wherein n=2, 3, 4, 5 or 6; C₃₋₈-cycloalkyl, wherein the cycloalkyl is unsubstituted or mono- or poly-substituted; or phenyl, which is unsubstituted or mono- or poly-substituted,
R² represents H or C(═O)—C₁₋₄-alkyl,
R³ represents methyl, ethyl, n-propyl, 2-propyl, n-butyl, tert-butyl, cyclopropyl, cyclopentyl or cyclohexyl, which, independently of one another, are unsubstituted or mono- or poly-substituted; phenyl, which is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy and dimethylamino; 1-naphthyl or 2-naphthyl, wherein the naphthyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy and dimethylamino; 9-phenanthrenyl, pyrrol-2-yl; pyridin-2-yl, pyridin-3-yl or pyridin-4-yl, wherein the pyridinyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; furan-2-yl or furan-3-yl, wherein the furanyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; thien-2-yl or thien-3-yl, wherein the thienyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; indol-2-yl, benzofuran-2-yl or benzofuran-3-yl,
R⁴, R⁵, R⁶ and R⁷ each independently of the others represents H, methyl, ethyl, n-propyl, 2-propyl, n-butyl, tert-butyl, CF₃, F, Cl, Br, I, CO₂H, CO₂-methyl, CO₂-ethyl, C(═O)CH₃ or NO₂, or
R⁶ and R⁷ form the hydrocarbon bridge —CH═CH—CH═CH—,
R⁸ represents C(═O)CH₃ and
R¹², R¹³ and R¹⁴ each independently of the others represents C₁₋₆-alkyl, which is straight-chained or branched, and unsubstituted or mono- or poly-substituted; C₃₋₈-cycloalkyl or CH₂—C₃₋₈-cycloalkyl, wherein the cycloalkyl is unsubstituted or mono- or poly-substituted; or phenyl, wherein the phenyl is unsubstituted or mono- or poly-substituted.

4. A pharmaceutical composition according to claim 3, wherein in formula I
R¹ represents methyl, n-butyl, 1,1,3,3-tetramethylbutyl, benzyl, 2-chlorobenzyl, 2-methoxybenzyl, CH₂CO₂CH₃, (CH₂)₆—NC, cyclopentyl, cyclohexyl, phenyl, 2,6-dimethylphenyl, 3-chlorophenyl or 3-chloro-4-fluorophenyl,
R² represents H or C(═O)CH₃,
R³ represents methyl, tert-butyl, cyclohexyl, phenyl, 2-methylphenyl, 3-methylphenyl, 4-methylphenyl, 2-trifluoromethylphenyl, 3-trifluoromethylphenyl, 4-trifluoromethylphenyl, 2-hydroxyphenyl, 2-methoxyphenyl, 3-hydroxyphenyl, 3-methoxyphenyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 2-nitrophenyl, 3-nitrophenyl, 4-nitrophenyl, 3-(4-chlorophenoxy)-phenyl, 2,4-dimethylphenyl, 2,3-dimethoxyphenyl, 3,4-dimethoxyphenyl, 3-methoxy-4-acetoxyphenyl, 2,3-dichlorophenyl, 2,4-dichlorophenyl, 2-chloro-4-fluorophenyl, 2-chloro-6-fluorophenyl, 4-bromo-2-fluorophenyl, 3,4,5-trimethoxyphenyl, 1-naphthyl, 2-ethoxy-naphth-1-yl, 4-dimethylamino-napth-1-yl, 9-phenanthrenyl, pyrrol-2-yl, N-methylpyrrol-2-yl, pyridin-2-yl, pyridin-3-yl, pyridin-4-yl, furan-2-yl, furan-3-yl, 5-methyl-furan-2-yl, 4,5-dimethyl-furan-2-yl, 5-hydroxymethyl-furan-2-yl, 5-acetoxymethyl-furan-2-yl, 5-carboxy-furan-2-yl, 5-[1,3]-dioxolan-furan-2-yl, 3-bromo-furan-2-yl, 5-bromo-furan-2-yl, 5-nitro-furan-2-yl, 5-(2-nitrophenyl)-furan-2-yl, 5-(2-chlorophenyl)-furan-2-yl, 5-(3-chlorophenyl)-furan-2-yl, 5-(3-chlorophenyl)-furan-3-yl, 5-(4-chlorophenyl)-furan-2-yl, benzo[b]furan-2-yl, thien-2-yl, thien-3-yl, 5-methyl-thien-2-yl, 5-carboxy-thien-2-yl, 3-bromo-thien-2-yl, 5-chloro-thien-2-yl or 5-methylsulfanyl-thien-2-yl,
R⁴ represents H, CH₃, Cl, Br or CO₂H,
R⁵ represents H, CH₃, C₂H₅ or Cl,
R⁶ represents H, CH₃, Cl, Br or NO₂,
R⁷ represents H, CH₃ or n-C₃H₇ and
R⁸ represents C(═O)CH₃.

5. A pharmaceutical composition according to claim 4, wherein in formula I R⁴ and R⁶ represent H, R⁵ represents H, CH₃ or C₂H₅ and R⁷ represents H or CH₃.

6. A pharmaceutical composition according to claim 5, wherein the compound is selected from the group consisting of:
tert-butyl-(7-methyl-2-pyridin-3-yl-imidazo[1,2-a]pyridin-3-yl)-amine,
cyclohexyl-(5,7-dimethyl-2-pyridin-4-yl-imidazo[1,2-a]pyridin-3-yl)-amine,
(5,7-dimethyl-2-pyridin-4-yl-imidazo[1,2-a]pyridin-3-yl)-(1,1,3,3-tetramethyl-butyl)-amine,
{6-[5,7-dimethyl-2-(1H-pyrrol-2-yl)-imidazo[1,2-a]pyridin-3-ylamino]-hexyl}-methylidyne-ammonium,
tert-butyl-[2-(2,3-dimethoxy-phenyl)-5,7-dimethyl-imidazo[1,2-a]pyridin-3-yl]-amine,
[2-(3,4-dimethoxy-phenyl)-7-methyl-imidazo[1,2-a]pyridin-3-yl]-(1, 1,3,3-tetramethyl-butyl)-amine,
cyclohexyl-(7-methyl-2-pyridin-4-yl-imidazo[1,2-a]pyridin-3-yl)-amine,
(2-furan-2-yl-5,7-dimethyl-imidazo[1,2-a]pyridin-3-yl)-(1,1,3,3-tetramethylbutyl)-amine,
(1,1,3,3-tetramethyl-butyl)-(2,5,7-trimethyl-imidazo[1,2-a]pyridin-3-yl)-amine,
cyclohexyl-(7-methyl-2-o-tolyl-imidazo[1,2-a]pyridin-3-yl)-amine,
cyclohexyl-(7-methyl-2-thiophen-2-yl-imidazo[1,2-a]pyridin-3-yl)-amine,
(5,7-dimethyl-2-pyridin-3-yl-imidazo[1,2-a]pyridin-3-yl)-(1,1,3,3-tetramethyl-butyl)-amine,
cyclohexyl-[7-methyl-2-(2-trifluoromethyl-phenyl)-imidazo[1,2-a]pyridin-3-yl]-amine,
tert-butyl-(2,5,7-trimethyl-imidazo[1,2-a]pyridin-3-yl)-amine,
(7-methyl-2-pyridin-3-yl-imidazo[1,2-a]pyridin-3-yl)-(1,1,3,3-tetramethylbutyl)-amine,
cyclohexyl-(7-methyl-2-naphthalen-1-yl-imidazo[1,2-a]pyridin-3-yl)-amine,
[2-(2-fluoro-phenyl)-7-methyl-imidazo[1,2-a]pyridin-3-yl]-(1,1,3,3-tetramethyl-butyl)-amine,
(2,7-dimethyl-imidazo[1,2-a]pyridin-3-ylamino)-acetic acid methyl ester,
methylidyne-[6-(7-methyl-2-pyridin-3-yl-imidazo[1,2-a]pyridin-3-ylamino)-hexyl]-ammonium,
3-(3-tert-butylamino-5,7-dimethyl-imidazo[1,2-a]pyridin-2-yl)-phenol,
cyclohexyl-[2-(2-fluoro-phenyl)-7-methyl-imidazo[1,2-a]pyridin-3-yl]-amine,
tert-butyl-(2-cyclohexyl-5,7-dimethyl-imidazo[1,2-a]pyridin-3-yl)-amine,
cyclohexyl-(7-methyl-2-pyridin-3-yl-imidazo[1,2-a]pyridin-3-yl)-amine,
3-(3-tert-butylamino-7-methyl-imidazo[1,2-a]pyridin-2-yl)-phenol,
tert-butyl-(2-furan-2-yl-5,7-dimethyl-imidazo[1,2-a]pyridin-3-yl)-amine,
cyclohexyl-(2-furan-2-yl-5,7-dimethyl-imidazo[1,2-a]pyridin-3-yl)-amine,
cyclohexyl-(5,7-dimethyl-2-pyridin-3-yl-imidazo[1,2-a]pyridin-3-yl)-amine,
(2-furan-2-yl-7-methyl-imidazo[1,2-a]pyridin-3-yl)-(1,1,3,3-tetramethylbutyl)-amine,
(7-methyl-2-pyridin-4-yl-imidazo[1,2-a]pyridin-3-yl)-(1,1,3,3-tetramethylbutyl)-amine,
butyl-(2-cyclohexyl-7-methyl-imidazo[1,2-a]pyridin-3-yl)-amine,
3-[5,7-dimethyl-3-(1,1,3,3-tetramethyl-butylamino)-imidazo[1,2-a]pyridin-2-yl]-phenol,
(2,6-dimethyl-phenyl)-(5,7-dimethyl-2-o-tolyl-imidazo[1,2-a]pyridin-3-yl)-amine,
tert-butyl-(7-methyl-2-naphthalen-1-yl-imidazo[1,2-a]pyridin-3-yl)-amine,
(2,6-dimethyl-phenyl)-[2-(2-fluoro-phenyl)-5,7-dimethyl-imidazo[1,2-a]-pyridin-3-yl]-amine,
cyclohexyl-(2,5,7-trimethyl-imidazo[1,2-a]pyridin-3-yl)-amine,
[5,7-dimethyl-2-(1H-pyrrol-2-yl)-imidazo[1,2-a]pyridin-3-yl]-(1,1,3,3-tetramethyl-butyl)-amine,
butyl-(2-cyclohexyl-5,7-dimethyl-imidazo[1,2-a]pyridin-3-yl)-amine,
(5,7-dimethyl-2-o-tolyl-imidazo[1,2-a]pyridin-3-yl)-(1,1,3,3-tetramethylbutyl)-amine,
[2-(2,3-dimethoxy-phenyl)-7-methyl-imidazo[1,2-a]pyridin-3-yl]-(2,6-dimethyl-phenyl)-amine,
(2,7-dimethyl-imidazo[1,2-a]pyridin-3-yl)-(1,1,3,3-tetramethyl-butyl)-amine,
[2-(5-[1,3]dioxolan-2-yl-furan-2-yl)-7-methyl-imidazo[1,2-a]pyridin-3-yl]-(1,1,3,3-tetramethyl-butyl)-amine,
[2-(3-bromo-thiophen-2-yl)-5,7-dimethyl-imidazo[1,2-a]pyridin-3-yl]-(2,6-dimethyl-phenyl)-amine,
(2,6-dimethyl-phenyl)-[2-(2-fluoro-phenyl)-7-methyl-imidazo[1,2-a]pyridin-3-yl]-amine,
(2-cyclohexyl-5,7-dimethyl-imidazo[1,2-a]pyridin-3-yl)-(1,1,3,3-tetramethylbutyl)-amine,
[6-(2-furan-2-yl-5,7-dimethyl-imidazo[1,2-a]pyridin-3-ylamino)-hexyl]-methylidyne-ammonium,
(7-methyl-2-o-tolyl-imidazo[1,2-a]pyridin-3-yl)-(1,1,3,3-tetramethyl-butyl)-amine,
[2-(2,3-dichloro-phenyl)-7-methyl-imidazo[1,2-a]pyridin-3-yl]-(2,6-dimethylphenyl)-amine,
[2-(2,3-dimethoxy-phenyl)-5,7-dimethyl-imidazo[1,2-a]pyridin-3-yl]-(2,6-dimethyl-phenyl)-amine,
butyl-[2-(2,3-dimethoxy-phenyl)-5,7-dimethyl-imidazo[1,2-a]pyridin-3-yl]-amine,
methylidyne-[6-(7-methyl-2-p-tolyl-imidazo[1,2-a]pyridin-3-ylamino)-hexyl]-ammonium,
tert-butyl-[5,7-dimethyl-2-(5-nitro-furan-2-yl)-imidazo[1,2-a]pyridin-3-yl]-amine,
acetic acid 5-(3-cyclohexylamino-5,7-dimethyl-imidazo[1,2-a]pyridin-2-yl)-furan-2-yl methyl ester,
[2-(2-methoxy-phenyl)-5,7-dimethyl-imidazo[1,2-a]pyridin-3-yl]-(1,1,3,3-tetramethyl-butyl)-amine,
[2-(3,4-dimethoxy-phenyl)-7-methyl-imidazo[1,2-a]pyridin-3-yl]-(2,6-dimethyl-phenyl)-amine,
3-(3-butylamino-7-methyl-imidazo[1,2-a]pyridin-2-yl)-phenol,
(2-benzofuran-2-yl-7-methyl-imidazo[1,2-a]pyridin-3-yl)-(1,1,3,3-tetramethyl-butyl)-amine,
(2-benzofuran-2-yl-5,7-dimethyl-imidazo[1,2-a]pyridin-3-yl)-(2,6-dimethylphenyl)-amine,
acetic acid 5-(3-cyclohexylamino-7-methyl-imidazo[1,2-a]pyridin-2-yl)-furan-2-yl methyl ester,
[6-(5,7-dimethyl-2-pyridin-3-yl-imidazo[1,2-a]pyridin-3-ylamino)-hexyl]-methylidyne-ammonium,
butyl-[2-(2-methoxy-phenyl)-7-methyl-imidazo[1,2-a]pyridin-3-yl]-amine,
{6-[2-(2-methoxy-phenyl)-5,7-dimethyl-imidazo[1,2-a]pyridin-3-ylamino]-hexyl}-methylidyne-ammonium,
{5-[5,7-dimethyl-3-(1,1,3,3-tetramethyl-butylamino)-imidazo[1,2-a]pyridin-2-yl]-furan-2-yl}-methanol,
(7-methyl-2-naphthalen-1-yl-imidazo[1,2-a]pyridin-3-yl)-(1,1,3,3-tetramethyl-butyl)-amine,
[5-(3-tert-butylamino-7-methyl-imidazo[1,2-a]pyridin-2-yl)-furan-2-yl]-methanol,
tert-butyl-[2-(5-[1,3]dioxolan-2-yl-furan-2-yl)-7-methyl-imidazo[1,2-a]-pyridin-3-yl]-amine,
(2-benzofuran-2-yl-5,7-dimethyl-imidazo[1,2-a]pyridin-3-yl)-(1,1,3,3-tetramethyl-butyl)-amine,
5-(3-tert-butylamino-5,7-dimethyl-imidazo[1,2-a]pyridin-2-yl)-furan-2-carboxylic acid,
tert-butyl-(2-furan-2-yl-7-methyl-imidazo[1,2-a]pyridin-3-yl)-amine,
cyclohexyl-(2,7-dimethyl-imidazo[1,2-a]pyridin-3-yl)-amine,
[2-(2,3-dichlorophenyl)-8-methyl-imidazo[1,2-a]pyridin-3-yl]-(1,1,3,3-tetramethyl-butyl)-amine,
(7-methyl-2-p-tolyl-imidazo[1,2-a]pyridin-3-yl)-(1,1,3,3-tetramethyl-butyl)-amine,
(2,6-dimethyl-phenyl)-[2-(2-methoxy-phenyl)-7-methyl-imidazo[1,2-a]-pyridin-3-yl]-amine,
3-(3-butylamino-5,7-dimethyl-imidazo[1,2-a]pyridin-2-yl)-phenol,
butyl-[2-(2,3-dimethoxy-phenyl)-7-methyl-imidazo[1,2-a]pyridin-3-yl]-amine,
{6-[5,7-dimethyl-2-(2-trifluoromethyl-phenyl)-imidazo[1,2-a]pyridin-3-ylamino]-hexyl}-methylidyne-ammonium,
tert-butyl-(7-methyl-2-pyridin-2-yl-imidazo[1,2-a]pyridin-3-yl)-amine,
cyclohexyl-(2-cyclohexyl-5,7-dimethyl-imidazo[1,2-a]pyridin-3-yl)-amine,
[2-(2,3-dichlorophenyl)-5,7-dimethyl-imidazo[1,2-a]pyridin-3-yl]-(2,6-dimethyl-phenyl)-amine,
(2,6-dimethyl-phenyl)-[2-(2-methoxy-phenyl)-5,7-dimethyl-imidazo[1,2-a]-pyridin-3-yl]-amine,
{2-[5-(2-chlorophenyl)-furan-2-yl]-7-methyl-imidazo[1,2-a]pyridin-3-yl}-(1,1,3,3-tetramethyl-butyl)-amine,
5-[7-methyl-3-(1,1,3,3-tetramethyl-butylamino)-imidazo[1,2-a]pyridin-2-yl]-furan-2-carboxylic acid,
cyclohexyl-[2-(2-methoxy-phenyl)-8-methyl-imidazo[1,2-a]pyridin-3-yl]-amine,
3-[7-methyl-3-(1,1,3,3-tetramethyl-butylamino)-imidazo[1,2-a]pyridin-2-yl]-phenol,
[2-(2,3-dichlorophenyl)-5,7-dimethyl-imidazo[1,2-a]pyridin-3-yl]-(1,1,3,3-tetramethyl-butyl)-amine,
[2-(2,4-dichlorophenyl)-7-methyl-imidazo[1,2-a]pyridin-3-yl]-(1,1,3,3-tetramethyl-butyl)-amine,
[2-(5-bromofuran-2-yl)-7-methyl-imidazo[1,2-a]pyridin-3-yl]-(1,1,3,3-tetramethyl-butyl)-amine,
5-(3-cyclohexylamino-5,7-dimethyl-imidazo[1,2-a]pyridin-2-yl)-furan-2-carboxylic acid,
[6-(2-cyclohexyl-5,7-dimethyl-imidazo[1,2-a]pyridin-3-ylamino)-hexyl]-methylidyne-ammonium,
[2-(2,4-dichlorophenyl)-5,7-dimethyl-imidazo[1,2-a]pyridin-3-yl]-(1,1,3,3-tetramethyl-butyl)-amine,
(2-benzofuran-2-yl-7-methyl-imidazo[1,2-a]pyridin-3-yl)-(2,6-dimethylphenyl)-amine,
5-(3-cyclohexylamino-7-methyl-imidazo[1,2-a]pyridin-2-yl)-furan-2-carboxylic acid,
{6-[2-(2-bromophenyl)-5,7-dimethyl-imidazo[1,2-a]pyridin-3-ylamino]-hexyl}-methylidyne-ammonium,
tert-butyl-(2-cyclohexyl-7-methyl-imidazo[1,2-a]pyridin-3-yl)-amine,
tert-butyl-(2,7-dimethyl-imidazo[1,2-a]pyridin-3-yl)-amine,
(5,7-dimethyl-2-p-tolyl-imidazo[1,2-a]pyridin-3-yl)-(1,1,3,3-tetramethylbutyl)-amine,
[2-(2,3-dichlorophenyl)-8-methyl-imidazo[1,2-a]pyridin-3-yl]-(2,6-dimethylphenyl)-amine,
methylidyne-[6-(7-methyl-2-o-tolyl-imidazo[1,2-a]pyridin-3-ylamino)-hexyl]-ammonium,
{2-[5-(3-chlorophenyl)-furan-2-yl]-7-methyl-imidazo[1,2-a]pyridin-3-yl}-(1,1,3,3-tetramethyl-butyl)-amine,
cyclohexyl-[7-methyl-2-(5-nitro-furan-2-yl)-imidazo[1,2-a]pyridin-3-yl]-amine,
[2-(2-bromophenyl)-7-methyl-imidazo[1,2-a]pyridin-3-yl]-cyclohexyl-amine,
[2-(2-methoxy-phenyl)-7-methyl-imidazo[1,2-a]pyridin-3-yl]-(1,1,3,3-tetramethyl-butyl)-amine,
{5-[7-methyl-3-(1,1,3,3-tetramethyl-butylamino)-imidazo[1,2-a]pyridin-2-yl]-furan-2-yl}-methanol,
(6-{2-[5-(2-chlorophenyl)-furan-2-yl]-5-methyl-imidazo[1,2-a]pyridin-3-ylamino}-hexyl)-methylidyne-ammonium,
cyclohexyl-[5,7-dimethyl-2-(5-nitro-furan-2-yl)-imidazo[1,2-a]pyridin-3-yl]-amine,
cyclohexyl-[2-(4,5-dimethyl-furan-2-yl)-7-methyl-imidazo[1,2-a]pyridin-3-yl]-amine,
[6-(5,7-dimethyl-2-o-tolyl-imidazo[1,2-a]pyridin-3-ylamino)-hexyl]-methylidyne-ammonium,
methylidyne-[6-(7-methyl-2-pyridin-2-yl-imidazo[1,2-a]pyridin-3-ylamino)-hexyl]-ammonium,
[2-(2,3-dimethoxy-phenyl)-5,7-dimethyl-imidazo[1,2-a]pyridin-3-yl]-(1,1,3,3-tetramethyl-butyl)-amine,
{6-[2-(3,4-dimethoxy-phenyl)-7-methyl-imidazo[1,2-a]pyridin-3-ylamino]-hexyl}-methylidyne-ammonium,
5-(3-tert-butylamino-7-methyl-imidazo[1,2-a]pyridin-2-yl)-thiophene-2-carboxylic acid,
cyclohexyl-(8-methyl-2-pyridin-4-yl-imidazo[1,2-a]pyridin-3-yl)-amine,
[2-(2,3-dichloro-phenyl)-6-methyl-imidazo[1,2-a]pyridin-3-yl]-(1,1,3,3-tetramethyl-butyl)-amine,
5-(3-butylamino-imidazo[1,2-a]pyrazin-2-yl)-thiophene-2-carboxylic acid,
cyclohexyl-(5,7-dimethyl-2-pyridin-2-yl-imidazo[1,2-a]pyridin-3-yl)-amine,
(2-benzofuran-2-yl-8-methyl-imidazo[1,2-a]pyridin-3-yl)-(1,1,3,3-tetramethyl-butyl)-amine,
{6-[2-(2-fluoro-phenyl)-5,7-dimethyl-imidazo[1,2-a]pyridin-3-ylamino]-hexyl}-methylidyne-ammonium,
[2-(2,3-dimethoxy-phenyl)-8-methyl-imidazo[1,2-a]pyridin-3-yl]-(1,1,3,3-tetramethyl-butyl)-amine,
methylidyne-[6-(7-methyl-2-phenanthren-9-yl-imidazo[1,2-a]pyridin-3-ylamino)-hexyl]-ammonium,
5-(3-tert-butylamino-imidazo[1,2-a]pyrazin-2-yl)-thiophene-2-carboxylic acid,
tert-butyl-(8-methyl-2-pyridin-2-yl-imidazo[1,2-a]pyridin-3-yl)-amine,
cyclohexyl-(2-furan-2-yl-8-methyl-imidazo[1,2-a]pyridin-3-yl)-amine,
methylidyne-[6-(7-methyl-2-naphthalen-1-yl-imidazo[1,2-a]pyridin-3-ylamino)-hexyl]-ammonium,
tert-butyl-(2-cyclohexyl-8-methyl-imidazo[1,2-a]pyridin-3-yl)-amine,
(6-methyl-2-pyridin-4-yl-imidazo[1,2-a]pyridin-3-yl)-(1,1,3,3-tetramethylbutyl)-amine,
tert-butyl-(6-methyl-2-pyridin-3-yl-imidazo[1,2-a]pyridin-3-yl)-amine,
(7-methyl-2-pyridin-3-yl-imidazo[1,2-a]pyrimidin-3-yl)-(1,1,3,3-tetramethylbutyl)-amine,
5-(3-tert-butylamino-6-methyl-imidazo[1,2-a]pyridin-2-yl)-thiophene-2-carboxylic acid,
[6-(5,7-dimethyl-2-naphthalen-1-yl-imidazo[1,2-a]pyridin-3-ylamino)-hexyl]-methylidyne-ammonium,
3-[3-(2,6-dimethyl-phenylamino)-5,7-dimethyl-imidazo[1,2-a]pyridin-2-yl]-phenol,
(2,6-dimethyl-phenyl)-(8-methyl-2-o-tolyl-imidazo[1,2-a]pyridin-3-yl)-amine,
{6-[2-(3-hydroxy-phenyl)-8-methyl-imidazo[1,2-a]pyridin-3-ylamino]-hexyl}-methylidyne-ammonium,
{5-[3-(2,6-dimethyl-phenylamino)-7-methyl-imidazo[1,2-a]pyrimidin-2-yl]-furan-2-yl}-methanol,
(8-methyl-2-p-tolyl-imidazo[1,2-a]pyridin-3-yl)-(1,1,3,3-tetramethyl-butyl)-amine,
[2-(2,4-dichlorophenyl)-6-methyl-imidazo[1,2-a]pyridin-3-yl]-(2,6-dimethylphenyl)-amine,
butyl-[2-(2,4-dichloro-phenyl)-6-methyl-imidazo[1,2-a]pyridin-3-yl]-amine,
butyl-[2-(4-dimethylamino-naphthalen-1-yl)-imidazo[1,2-a]pyrazin-3-yl]-amine,
{6-[2-(2-bromo-phenyl)-7-methyl-imidazo[1,2-a]pyridin-3-ylamino]-hexyl}-methylidyne-ammonium,
butyl-[2-(2-methoxy-phenyl)-6-methyl-imidazo[1,2-a]pyridin-3-yl]-amine,
(2-cyclohexyl-8-methyl-imidazo[1,2-a]pyridin-3-yl)-(1,1,3,3-tetramethylbutyl)-amine,
cyclohexyl-(7-methyl-2-pyridin-2-yl-imidazo[1,2-a]pyridin-3-yl)-amine,
cyclohexyl-(2-furan-2-yl-7-methyl-imidazo[1,2-a]pyridin-3-yl)-amine,
(2-cyclohexyl-5,7-dimethyl-imidazo[1,2-a]pyridin-3-ylamino)-acetic acid methyl ester,
N-(2-furan-2-yl-5,7-dimethyl-imidazo[1,2-a]pyridin-3-yl)-N-(1,1,3,3-tetramethyl-butyl)-acetamide,
N-tert-butyl-N-(7-methyl-2-pyridin-3-yl-imidazo[1,2-a]pyridin-3-yl)-acetamide,
N-tert-butyl-N-(2-furan-2-yl-5,7-dimethyl-imidazo[1,2-a]pyridin-3-yl)-acetamide,
N-(5,7-dimethyl-2-pyridin-4-yl-imidazo[1,2-a]pyridin-3-yl)-N-(1,1,3,3-tetramethyl-butyl)-acetamide,
N-(5,7-dimethyl-2-pyridin-3-yl-imidazo[1,2-a]pyridin-3-yl)-N-(1,1,3,3-tetramethyl-butyl)-acetamide,
N-(2,6-dimethyl-phenyl)-N-(5,7-dimethyl-2-o-tolyl-imidazo[1,2-a]pyridin-3-yl)-acetamide,
N-(2-furan-2-yl-7-methyl-imidazo[1,2-a]pyridin-3-yl)-N-(1,1,3,3-tetramethylbutyl)-acetamide,
N-(1,1,3,3-tetramethyl-butyl)-N-(2,5,7-trimethyl-imidazo[1,2-a]pyridin-3-yl)-acetamide,
N-cyclohexyl-N-(7-methyl-2-thiophen-2-yl-imidazo[1,2-a]pyridin-3-yl)-acetamide,
N-tert-butyl-N-(2,5,7-trimethyl-imidazo[1,2-a]pyridin-3-yl)-acetamide,
5-[3-(acetyl-tert-butyl-amino)-imidazo[1,2-a]pyrazin-2-yl]-thiophene-2-carboxylic acid,
5-{3-[acetyl-(1,1,3,3-tetramethyl-butyl)-amino]-5,7-dimethyl-imidazo[1,2-a]-pyridin-2-yl}-furan-2-carboxylic acid,
N-[2-(5-hydroxymethyl-furan-2-yl)-5,7-dimethyl-imidazo[1,2-a]pyridin-3-yl]-N-(1,1,3,3-tetramethyl-butyl)-acetamide,
N-[2-(3-bromo-thiophen-2-yl)-5,7-dimethyl-imidazo[1,2-a]pyridin-3-yl]-N-(2,6-dimethyl-phenyl)-acetamide,
N-tert-butyl-N-(2-cyclohexyl-5,7-dimethyl-imidazo[1,2-a]pyridin-3-yl)-acetamide,
acetic acid 5-[3-(acetyl-cyclohexyl-amino)-5,7-dimethyl-imidazo[1,2-a]-pyridin-2-yl]-furan-2-yl methyl ester,
{6-[acetyl-(2-furan-2-yl-5,7-dimethyl-imidazo[1,2-a]pyridin-3-yl)-amino]-hexyl}-methylidyne-ammonium,
N-[2-(2,3-dichloro-phenyl)-7-methyl-imidazo[1,2-a]pyridin-3-yl]-N-(2,6-dimethyl-phenyl)-acetamide,
N-[2-(3-bromo-thiophen-2-yl)-imidazo[1,2-a]pyridin-3-yl]-N-cyclohexyl-acetamide,
N-(5,7-dimethyl-2-p-tolyl-imidazo[1,2-a]pyridin-3-yl)-N-(1,1,3,3-tetramethyl-butyl)-acetamide,
N-cyclohexyl-N-(7-methyl-2-pyridin-4-yl-imidazo[1,2-a]pyridin-3-yl)-acetamide,
N-cyclohexyl-N-[7-methyl-2-(2-trifluoromethyl-phenyl)-imidazo[1,2-a]-pyridin-3-yl]-acetamide,
N-(6,8-dibromo-2-furan-2-yl-imidazo[1,2-a]pyridin-3-yl)-N-(1,1,3,3-tetramethyl-butyl)-acetamide,
N-(7-methyl-2-pyridin-4-yl-imidazo[1,2-a]pyridin-3-yl).—N-(1,1,3,3-tetramethyl-butyl)-acetamide,
acetic acid 5-[3-(acetyl-cyclohexyl-amino)-7-methyl-imidazo[1,2-a]pyridin-2-yl]-furan-2-yl methyl ester,
N-(7-methyl-2-pyridin-3-yl-imidazo[1,2-a]pyridin-3-yl)-N-(1,1,3,3-tetramethyl-butyl)-acetamide,
N-[2-(2,3-dichloro-phenyl)-5,7-dimethyl-imidazo[1,2-a]pyridin-3-yl]-N-(2,6-dimethyl-phenyl)-acetamide,
N-cyclohexyl-N-[5,7-dimethyl-2-(5-methyl-furan-2-yl)-imidazo[1,2-a]-pyridin-3-yl]-acetamide,
N-butyl-N-[2-(2,3-dimethoxy-phenyl)-5,7-dimethyl-imidazo[1,2-a]pyridin-3-yl]-acetamide,
N-[2-(2-methoxy-phenyl)-5,7-dimethyl-imidazo[1,2-a]pyridin-3-yl]-N-(1,1,3,3-tetramethyl-butyl)-acetamide,
N-cyclohexyl-N-[5,7-dimethyl-2-(5-nitro-furan-2-yl)-imidazo[1,2-a]pyridin-3-yl]-acetamide,
[acetyl-(2,7-dimethyl-imidazo[1,2-a]pyridin-3-yl)-amino]-acetic acid methyl ester,
N-cyclohexyl-N-(2,5,7-trimethyl-imidazo[1,2-a]pyridin-3-yl)-acetamide,
5-{3-[acetyl-(1,1,3,3-tetramethyl-butyl)-amino]-imidazo[1,2-a]pyridin-2-yl}-thiophene-2-carboxylic acid,
N-[2-(2,4-dichloro-phenyl)-5,7-dimethyl-imidazo[1,2-a]pyridin-3-yl]-N-(1,1,3,3-tetramethyl-butyl)-acetamide,
N-cyclohexyl-N-[7-methyl-2-(5-nitro-furan-2-yl)-imidazo[1,2-a]pyridin-3-yl]-acetamide,
N-(2-tert-butyl-5,7-dimethyl-imidazo[1,2-a]pyridin-3-yl)-N-(2,6-dimethylphenyl)-acetamide,
N-(2,6-dimethyl-phenyl)-N-[2-(2-methoxy-phenyl)-5,7-dimethyl-imidazo[1,2-a]pyridin-3-yl]-acetamide,
N-[2-(3-hydroxy-phenyl)-5,7-dimethyl-imidazo[1,2-a]pyridin-3-yl]-N-(1,1,3,3-tetramethyl-butyl)-acetamide,
N-(2,6-dimethyl-phenyl)-N-[2-(2-fluoro-phenyl)-5,7-dimethyl-imidazo[1,2-a]pyridin-3-yl]-acetamide,
5-[3-(acetyl-tert-butyl-amino)-5-methyl-imidazo[1,2-a]pyridin-2-yl]-thiophene-2-carboxylic acid,
N-(2,6-dimethyl-phenyl)-N-[2-(2-methoxy-phenyl)-7-methyl-imidazo[1,2-a]-pyridin-3-yl]-acetamide,
N-(7-methyl-2-p-tolyl-imidazo[1,2-a]pyridin-3-yl)-N-(1,1,3,3-tetramethylbutyl)-acetamide,
5-{3-[acetyl-(1,1,3,3-tetramethyl-butyl)-amino]-7-methyl-imidazo[1,2-a]-pyridin-2-yl}-furan-2-carboxylic acid,
N-cyclohexyl-N-(7-methyl-2-pyridin-3-yl-imidazo[1,2-a]pyridin-3-yl)-acetamide,
N-[2-(5-[1,3]dioxolan-2-yl-furan-2-yl)-5,7-dimethyl-imidazo[1,2-a]pyridin-3-yl]-N-(1,1,3,3-tetramethyl-butyl)-acetamide,
N-(2-benzofuran-2-yl-5,7-dimethyl-imidazo[1,2-a]pyridin-3-yl)-N-(2,6-dimethyl-phenyl)-acetamide,
N-tert-butyl-N-(2-furan-2-yl-7-methyl-imidazo[1,2-a]pyridin-3-yl)-acetamide,
N-tert-butyl-N-(7-methyl-2-pyridin-2-yl-imidazo[1,2-a]pyridin-3-yl)-acetamide,
N-cyclohexyl-N-(5,7-dimethyl-2-pyridin-3-yl-imidazo[1,2-a]pyridin-3-yl)-acetamide,
N-[2-(2,3-dichloro-phenyl)-5,7-dimethyl-imidazo[1,2-a]pyridin-3-yl]-N-(1,1,3,3-tetramethyl-butyl)-acetamide,
N-[2-(2,3-dimethoxy-phenyl)-5,7-dimethyl-imidazo[1,2-a]pyridin-3-yl]-N-(1,1,3,3-tetramethyl-butyl)-acetamide,
N-{2-[3-(4-chloro-phenoxy)-phenyl]-imidazo[1,2-a]pyridin-3-yl}-N-(2,6-dimethyl-phenyl)-acetamide,
N-[2-(5-[1,3]dioxolan-2-yl-furan-2-yl)-7-methyl-imidazo[1,2-a]pyridin-3-yl]-N-(1,1,3,3-tetramethyl-butyl)-acetamide,
5-[3-(acetyl-cyclohexyl-amino)-5,7-dimethyl-imidazo[1,2-a]pyridin-2-yl]-furan-2-carboxylic acid,
N-tert-butyl-N-[7-methyl-2-(5-nitro-furan-2-yl)-imidazo[1,2-a]pyridin-3-yl]-acetamide,
N-[2-(2-methoxy-phenyl)-7-methyl-imidazo[1,2-a]pyridin-3-yl]-N-(1,1,3,3-tetramethyl-butyl)-acetamide,
N-[2-(5-methyl-furan-2-yl)-imidazo[1,2-a]pyridin-3-yl]-N-(1,1,3,3-tetramethyl-butyl)-acetamide,
5-[3-(acetyl-tert-butyl-amino)-7-methyl-imidazo[1,2-a]pyridin-2-yl]-furan-2-carboxylic acid,
N-[2-(4,5-dimethyl-furan-2-yl)-imidazo[1,2-a]pyridin-3-yl]-N-(1,1,3,3-tetramethyl-butyl)-acetamide,
N-cyclohexyl-N-(2-furan-2-yl-5,7-dimethyl-imidazo[1,2-a]pyridin-3-yl)-acetamide,
N-tert-butyl-N-(7-methyl-2-naphthalen-1-yl-imidazo[1,2-a]pyridin-3-yl)-acetamide,
5-{3-[acetyl-(1,1,3,3-tetramethyl-butyl)-amino]-imidazo[1,2-a]pyrazin-2-yl}-thiophene-2-carboxylic acid,
N-butyl-N-(2-o-tolyl-imidazo[1,2-a]pyrimidin-3-yl)acetamide,
N-[2-(3,4-dimethoxy-phenyl)-7-methyl-imidazo[1,2-a]pyridin-3-yl]-N-(2,6-dimethyl-phenyl)-acetamide,
N-tert-butyl-N-(7-methyl-2-phenanthren-9-yl-imidazo[1,2-a]pyridin-3-yl)-acetamide,
N-(2,6-dimethyl-phenyl)-N-[2-(2-fluoro-phenyl)-7-methyl-imidazo[1,2-a]-pyridin-3-yl]-acetamide,
N-[2-(2-methoxy-phenyl)-8-methyl-imidazo[1,2-a]pyridin-3-yl]-N-(1,1,3,3-tetramethyl-butyl)-acetamide,
N-(2,6-dimethyl-phenyl)-N-[2-(3-hydroxy-phenyl)-5,7-dimethyl-imidazo[1,2-a]pyridin-3-yl]-acetamide,
N-(2-tert-butyl-7-methyl-imidazo[1,2-a]pyridin-3-yl)-N-(2,6-dimethylphenyl)-acetamide,
acetic acid 4-{3-[acetyl-(2,6-dimethyl-phenyl)-amino]-6-bromo-8-methyl-imidazo[1,2-a]pyridin-2-yl}-2-methoxy-phenyl ester,
N-tert-butyl-N-[2-(5-[1,3]dioxolan-2-yl-furan-2-yl)-7-methyl-imidazo[1,2-a]-pyridin-3-yl]-acetamide,
[6-(acetyl-{7-methyl-2-[5-(2-nitro-phenyl)-furan-2-yl]-imidazo[1,2-a]pyridin-3-yl}-amino)-hexyl]-methylidyne-ammonium,
N-(2-benzofuran-2-yl-7-methyl-imidazo[1,2-a]pyridin-3-yl)-N-(1,1,3,3-tetramethyl-butyl)-acetamide,
N-(2-benzofuran-2-yl-5,7-dimethyl-imidazo[1,2-a]pyridin-3-yl)-N-(1,1,3,3-tetramethyl-butyl)-acetamide,
5-[3-(acetyl-tert-butyl-amino)-imidazo[1,2-a]pyridin-2-yl]-thiophene-2-carboxylic acid,
N-(2-cyclohexyl-5,7-dimethyl-imidazo[1,2-a]pyridin-3-yl)-N-(1,1,3,3-tetramethyl-butyl)-acetamide,
N-tert-butyl-N-[2-(5-methyl-furan-2-yl)-imidazo[1,2-a]pyridin-3-yl]-acetamide,
N-tert-butyl-N-[2-(5-methylsulfanyl-thiophen-2-yl)-imidazo[1,2-a]pyrimidin-3-yl]-acetamide,
N-[2-(4,5-dimethyl-furan-2-yl)-5-methyl-imidazo[1,2-a]pyridin-3-yl]-N-(1,1,3,3-tetramethyl-butyl)-acetamide,
N-butyl-N-[2-(2,4-dichloro-phenyl)-imidazo[1,2-a]pyrimidin-3-yl]-acetamide,
N-[2-(3-bromo-thiophen-2-yl)-6-methyl-imidazo[1,2-a]pyridin-3-yl]-N-cyclohexyl-acetamide,
5-{3-[acetyl-(2,6-dimethyl-phenyl)-amino]-6-methyl-imidazo[1,2-a]pyridin-2-yl}-thiophene-2-carboxylic acid,
N-butyl-N-[2-(2,3-dimethoxy-phenyl)-7-methyl-imidazo[1,2-a]pyridin-3-yl]-acetamide,
N-tert-butyl-N-[2-(2,3-dichloro-phenyl)-imidazo[1,2-a]pyridin-3-yl]-acetamide,
N-(2-furan-2-yl-5-propyl-imidazo[1,2-a]pyridin-3-yl)-N-(1,1,3,3-tetramethylbutyl)-acetamide,
5-[3-(acetyl-cyclohexyl-amino)-imidazo[1,2-a]pyridin-2-yl]-thiophene-2-carboxylic acid,
5-{3-[acetyl-(1,1,3,3-tetramethyl-butyl)-amino]-8-methyl-imidazo[1,2-a]-pyridin-2-yl}-furan-2-carboxylic acid,
3-(acetyl-butyl-amino)-2-pyridin-2-yl-imidazo[1,2-a]pyridine-8-carboxylic acid,
{6-[acetyl-(5,7-dimethyl-2-pyridin-3-yl-imidazo[1,2-a]pyridin-3-yl)-amino]-hexyl}-methylidyne-ammonium,
N-tert-butyl-N-[2-(5-methylsulfanyl-thiophen-2-yl)-imidazo[1,2-a]pyrazin-3-yl]-acetamide,
5-[3-(acetyl-cyclohexyl-amino)-5-methyl-imidazo[1,2-a]pyridin-2-yl]-thiophene-2-carboxylic acid,
N-[2-(5-methylsulfanyl-thiophen-2-yl)-imidazo[1,2-a]pyrazin-3-y]-N-(1,1,3,3-tetramethyl-butyl)-acetamide,
N-[2-(2,3-dichloro-phenyl)-5-methyl-imidazo[1,2-a]pyridin-3-yl]-N-(2,6-dimethyl-phenyl)-acetamide,
N-butyl-N-[2-(2-methoxy-phenyl)-imidazo[1,2-a]pyridin-3-yl]-acetamide,
(6-{acetyl-[2-(2-methoxy-phenyl)-6-nitro-imidazo[1,2-a]pyridin-3-yl]-amino}-hexyl)-methylidyne-ammonium,
N-(2-benzofuran-2-yl-7-methyl-imidazo[1,2-a]pyridin-3-yl)-N-(2,6-dimethylphenyl)-acetamide,
(6-{acetyl-[2-(2-methoxy-phenyl)-5,7-dimethyl-imidazo[1,2-a]pyridin-3-yl]-amino}-hexyl)-methylidyne-ammonium,
{6-[acetyl-(7-methyl-2-pyridin-3-yl-imidazo[1,2-a]pyridin-3-yl)-amino]-hexyl}-methylidyne-ammonium,
N-(6-methyl-2-p-tolyl-imidazo[1,2-a]pyridin-3-yl)-N-(1,1,3,3-tetramethylbutyl)-acetamide,
acetic acid 5-{3-[acetyl-(2,6-dimethyl-phenyl)-amino]-5,7-dimethyl-imidazo[1,2-a]pyridin-2-yl}-furan-2-yl methyl ester,
{acetyl-[2-(3-hydroxy-phenyl)-6-methyl-imidazo[1,2-a]pyridin-3-yl]-amino}-acetic acid methyl ester,
N-tert-butyl-N-[2-(2-trifluoromethyl-phenyl)-imidazo[1,2-a]pyridin-3-yl]-acetamide,
N-butyl-N-[2-(2-chloro-4-fluoro-phenyl)-imidazo[1,2-a]pyridin-3-yl]-acetamide,
N-[2-(2,4-dichloro-phenyl)-imidazo[1,2-a]pyridin-3-yl]-N-(2,6-dimethylphenyl)-acetamide,
5-{3-[acetyl-(1,1,3,3-tetramethyl-butyl)-amino]-7-methyl-imidazo[1,2-a]-pyrimidin-2-yl}-furan-2-carboxylic acid,
acetic acid 5-{3-[acetyl-(1,1,3,3-tetramethyl-butyl)-amino]-imidazo[1,2-a]-pyrimidin-2-yl}-furan-2-yl methyl ester,
N-(2,7-dimethyl-imidazo[1,2-a]pyridin-3-yl)-N-(1,1,3,3-tetramethyl-butyl)-acetamide,
acetic acid 4-[3-(acetyl-cyclohexyl-amino)-5-amino-7-chloro-imidazo[1,2-a]-pyrimidin-2-yl]-2-methoxy-phenyl ester,
acetic acid 4-[3-(acetyl-cyclohexyl-amino)-5,7-dimethyl-imidazo[1,2-a]-pyrimidin-2-yl]-2-methoxy-phenyl ester,
N-[6-bromo-2-(2-chloro-6-fluoro-phenyl)-8-methyl-imidazo[1,2-a]pyridin-3-yl]-N-cyclohexyl-acetamide,
N-[2-(2-chloro-6-fluoro-phenyl)-5-methyl-imidazo[1,2-a]pyridin-3-yl]-N-cyclohexyl-acetamide,
N-butyl-N-[2-(2,3-dichloro-phenyl)-imidazo[1,2-a]pyrimidin-3-yl]-acetamide,
N-[2-(5-chloro-thiophen-2-yl)-imidazo[1,2-a]pyrimidin-3-yl]-N-(1,1,3,3-tetramethyl-butyl)-acetamide,
[acetyl-(2-cyclohexyl-7-methyl-imidazo[1,2-a]pyridin-3-yl)-amino]-acetic acid methyl ester,
N-tert-butyl-N-[2-(2-chloro-6-fluoro-phenyl)-imidazo[1,2-a]pyridin-3-yl]-acetamide,
N-cyclohexyl-N-(5,7-dimethyl-2-pyridin-2-yl-imidazo[1,2-a]pyridin-3-yl)-acetamide,
acetic acid 5-[3-(acetyl-cyclohexyl-amino)-5-methyl-imidazo[1,2-a]pyridin-2-yl]-furan-2-yl methyl ester,
N-(2,6-dimethyl-phenyl)-N-[6-methyl-2-(2-trifluoromethyl-phenyl)-imidazo[1,2-a]pyridin-3-yl]-acetamide,
N-cyclohexyl-N-(2-furan-2-yl-7-methyl-imidazo[1,2-a]pyridin-3-yl)-acetamide,
N-cyclohexyl-N-(7-methyl-2-pyridin-2-yl-imidazo[1,2-a]pyridin-3-yl)-acetamide,
N-cyclohexyl-N-[2-(5-[1,3]dioxolan-2-yl-furan-2-yl)-5-methyl-imidazo[1,2-a]pyridin-3-yl]-acetamide,
N-tert-butyl-N-(5-propyl-2-pyridin-3-yl-imidazo[1,2-a]pyridin-3-yl)-acetamide,
N-tert-butyl-N-[2-(5-methyl-thiophen-2-yl)-imidazo[1,2-a]pyrimidin-3-yl]-acetamide,
3-[acetyl-(1,1,3,3-tetramethyl-butyl)-amino]-2-furan-2-yl-imidazo[1,2-a]-pyridine-8-carboxylic acid,
N-tert-butyl-N-[2-(4,5-dimethyl-furan-2-yl)-6-methyl-imidazo[1,2-a]pyridin-3-yl]-acetamide,
N-{2-[3-(4-chloro-phenoxy)-phenyl]-imidazo[1,2-a]pyridin-3-yl}-N-cyclohexyl-acetamide,
acetic acid 4-[3-(acetyl-cyclohexyl-amino)-imidazo[1,2-a]pyrimidin-2-yl]-2-methoxy-phenyl ester,
N-[2-(5-bromo-furan-2-yl)-8-methyl-imidazo[1,2-a]pyridin-3-yl]-N-(1,1,3,3-tetramethyl-butyl)-acetamide,
N-(2,6-dimethyl-phenyl)-N-[2-(3-hydroxy-phenyl)-5,7-dimethyl-imidazo[1,2-a]pyrimidin-3-yl]-acetamide,
N-cyclohexyl-N-[2-(2,3-dichloro-phenyl)-imidazo[1,2-a]pyridin-3-yl]-acetamide,
N-cyclohexyl-N-[2-(2,4-dichloro-phenyl)-5-methyl-imidazo[1,2-a]pyridin-3-yl]-acetamide,
N-cyclohexyl-N-[2-(2,4-dichloro-phenyl)-imidazo[1,2-a]pyrimidin-3-yl]-acetamide,
[acetyl-(2-o-tolyl-imidazo[1,2-a]pyrazin-3-yl)-amino]-acetic acid methyl ester,
N-tert-butyl-N-(6,8-dichloro-2-thiophen-2-yl-imidazo[1,2-a]pyridin-3-yl)-acetamide,
N-tert-butyl-N-(5-propyl-2-thiophen-2-yl-imidazo[1,2-a]pyridin-3-yl)-acetamide,
{6-[acetyl-(7-methyl-2-p-tolyl-imidazo[1,2-a]pyridin-3-yl)-amino]-hexyl}-methylidyne-ammonium,
N-butyl-N-(6-methyl-2-p-tolyl-imidazo[1,2-a]pyridin-3-yl)-acetamide,
(6-{acetyl-[2-(2-methoxy-phenyl)-7-methyl-imidazo[1,2-a]pyridin-3-yl]-amino}-hexyl)-methylidyne-ammonium,
5-{3-[acetyl-(1,1,3,3-tetramethyl-butyl)-amino]-6-methyl-imidazo[1,2-a]pyridin-2-yl}-furan-2-carboxylic acid,
N-butyl-N-[2-(3,4,5-trimethoxy-phenyl)-imidazo[1,2-a]pyrazin-3-yl]acetamide,
N-butyl-N-[2-(3-hydroxy-phenyl)-imidazo[1,2-a]pyrimidin-3-yl]-acetamide,
[acetyl-(2-o-tolyl-imidazo[1,2-a]pyrimidin-3-yl)-amino]-acetic acid methyl ester,
N-(2-benzofuran-2-yl-8-methyl-imidazo[1,2-a]pyridin-3-yl)-N-(1,1,3,3-tetramethyl-butyl)-acetamide,
N-butyl-N-(7-methyl-2-p-tolyl-imidazo[1,2-a]pyrimidin-3-yl)-acetamide,
N-tert-butyl-N-(6,8-dibromo-2-methyl-imidazo[1,2-a]pyridin-3-yl)-acetamide,
{6-[acetyl-(5,7-dimethyl-2-pyridin-2-yl-imidazo[1,2-a]pyridin-3-yl)-amino]-hexyl}-methylidyne-ammonium,
N-tert-butyl-N-[2-(2-ethoxy-naphthalen-1-yl)-7-methyl-imidazo[1,2-a]-pyridin-3-yl]-acetamide,
N-tert-butyl-N-[2-(2-chloro-4-fluoro-phenyl)-imidazo[1,2-a]pyridin-3-yl]-acetamide,
cyclohexyl-[7-methyl-2-(2-trifluoromethyl-phenyl)-imidazo[1,2-a]pyridin-3-yl]-amine hydrochloride,
tert-butyl-(2-furan-2-yl-5,7-dimethyl-imidazo[1,2-a]pyridin-3-yl)-amine hydrochloride,
tert-butyl-(7-methyl-2-phenyl-imidazo[1,2-a]pyridin-3-yl)-amine hydrochloride,
cyclohexyl-(5,7-dimethyl-2-pyridin-4-yl-imidazo[1,2-a]pyridin-3-yl)-amine hydrochloride,
(2-furan-2-yl-5,7-dimethyl-imidazo[1,2-a]pyridin-3-yl)-(1,1,3,3-tetramethylbutyl)-amine hydrochloride,
tert-butyl-(2,5,7-trimethyl-imidazo[1,2-a]pyridin-3-yl)-amine hydrochloride,
[2-(2-fluorophenyl)-7-methyl-imidazo[1,2-a]pyridin-3-yl]-(1,1,3,3-tetramethyl-butyl)-amine hydrochloride,
cyclohexyl-(7-methyl-2-phenyl-imidazo[1,2-a]pyridin-3-yl)-amine hydrochloride,
(2-furan-2-yl-7-methyl-imidazo[1,2-a]pyrimidin-3-yl)-(1,1,3,3-tetramethylbutyl)-amine hydrochloride,
tert-butyl-[2-(4-nitro-phenyl)-imidazo[1,2-a]pyrazin-3-yl]-amine hydrochloride,
N-{2-[3-(4-chlorophenoxy)-phenyl]-imidazo[1,2-a]pyridin-3-yl}-N-cyclohexyl-acetamide hydrochloride,
N-cyclohexyl-N-(7-methyl-2-o-tolyl-imidazo[1,2-a]pyridin-3-yl)-acetamide hydrochloride,
N-(2,6-dimethyl-phenyl)-N-[2-(2,4-dimethyl-phenyl)-5-methyl-imidazo[1,2-a]pyridin-3-yl]-acetamide hydrochloride,
1-acetyl-3-(acetyl-cyclohexyl-amino)-7-methyl-2-o-tolyl-imidazo[1,2-a]-pyridin-1-ium) chloride hydrochloride,
cyclohexyl-(2-furan-3-yl-5,7-dimethyl-imidazo[1,2-a]pyridin-3-yl)-amine hydrochloride,
cyclopentyl-(2-furan-3-yl-5,7-dimethyl-imidazo[1,2-a]pyridin-3-yl)-amine hydrochloride,
[2-(4-bromo-2-fluoro-phenyl)-5,7-dimethyl-imidazo[1,2-a]pyridin-3-yl]cyclopentyl-amine hydrochloride,
cyclopentyl-{5,7-dimethyl-2-[5-(2-nitro-phenyl)-furan-2-yl]-imidazo[1,2-a]-pyridin-3-yl}-amine hydrochloride,
{2-[5-(4-chlorophenyl)-furan-2-yl]-5,7-dimethyl-imidazo[1,2-a]pyridin-3-yl}-cyclopentyl-amine hydrochloride,
cyclopentyl-(2-furan-3-yl-5,7-dimethyl-imidazo[1,2-a]pyridin-3-yl)-amine hydrochloride,
(2-furan-3-yl-5,7-dimethyl-imidazo[1,2-a]pyridin-3-yl)-(1,1,3,3-tetramethylbutyl)-amine hydrochloride,
benzyl-(7-methyl-2-thiophen-3-yl-imidazo[1,2-a]pyridin-3-yl)-amine hydrochloride,
cyclohexyl-(2-furan-3-yl-5,7-dimethyl-imidazo[1,2-a]pyridin-3-yl)-amine hydrochloride,
(2-furan-3-yl-7-methyl-imidazo[1,2-a]pyridin-3-yl)-(1,1,3,3-tetramethylbutyl)-amine hydrochloride,
(5,7-dimethyl-2-thiophen-3-yl-imidazo[1,2-a]pyridin-3-yl)-(1,1,3,3-tetramethyl-butyl)-amine hydrochloride,
[7-ethyl-2-(5-nitro-furan-2-yl)-imidazo[1,2-a]pyridin-3-yl]-(2-methoxybenzyl)-amine,
(2-chlorobenzyl)-[7-ethyl-2-(5-nitro-furan-2-yl)-imidazo[1,2-a]pyridin-3-yl]-amine,
[7-ethyl-2-(5-methyl-furan-2-yl)-imidazo[1,2-a]pyridin-3-yl]-(2-methoxybenzyl)-amine,
(2-chlorobenzyl)-(7-ethyl-2-furan-2-yl-imidazo[1,2-a]pyridin-3-yl)-amine,
(3-chloro-4-fluorophenyl)-[7-ethyl-2-(5-methyl-furan-2-yl)-imidazo[1,2-a]-pyridin-3-yl]-amine,
(2-benzofuran-2-yl-7-ethyl-imidazo[1,2-a]pyridin-3-yl)-(3-chloro-4-fluorophenyl)-amine,
(2-benzofuran-2-yl-7-ethyl-imidazo[1,2-a]pyridin-3-yl)-(3-chlorophenyl)-amine,
(3-chloro-4-fluorophenyl)-{2-[5-(3-chloro-phenyl)-furan-2-yl]-7-ethyl-imidazo[1,2-a]pyridin-3-yl}-amine,
(3-chloro-4-fluorophenyl)-{2-[5-(2-chlorophenyl)-furan-2-yl]-7-ethyl-imidazo[1,2-a]pyridin-3-yl}-amine,
(3-chloro-4-fluorophenyl)-[2-(4,5-dimethyl-furan-2-yl)-7-ethyl-imidazo[1,2-a]pyridin-3-yl]-amine.

7. A pharmaceutical composition for treating one or more diseases selected from the group consisting of multiple sclerosis, Parkinson's disease, Alzheimer's disease, Huntington's disease, cerebral ischaemia, diabetes, meningitis, and arteriosclerosis, or for healing wounds, the pharmaceutical composition comprising an effective amount of a compound of formula I, or a pharmaceutically acceptable salt thereof, and a pharmaceutically acceptable excipient, 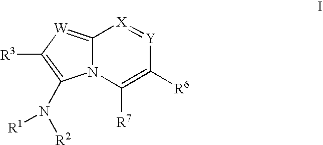
wherein in formula I,
X represents CR⁴ or N,
Y represents CR⁵ or N and
X and Y do not simultaneously represent N,
W represents N or NR⁸,
R¹ represents C₁₋₁₂-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; heterocyclyl, which is saturated or unsaturated, and unsubstituted or mono- or poly-substituted; aryl, which is unsubstituted or mono- or poly-substituted; heteroaryl, which is unsubstituted or mono- or poly-substituted; C₁₋₈-alkyl-aryl or C₁₋₈-alkyl-heteroaryl, wherein the alkyl is straight-chained or branched, saturated or unsaturated, and unsubstituted or mono- or poly-substituted, the aryl is unsubstituted or mono- or poly-substituted, and the heteroaryl is unsubstituted or mono- or poly-substituted,
R² represents hydrogen or C(═O)R⁹,
R³ represents C₁₋₈-alkyl, which is straight-chained or branched, saturated or unsaturated, and unsubstituted or mono- or poly-substituted; C₃₋₈-cycloalkyl, wherein the cycloalkyl is saturated or unsaturated, and unsubstituted or mono- or poly-substituted; heterocyclyl, which is saturated or unsaturated, and unsubstituted or mono- or poly-substituted; aryl, which is unsubstituted or mono- or poly-substituted; heteroaryl, which is unsubstituted or mono- or poly-substituted; C₁₋₈-alkyl-C₃₋₈-cycloalkyl, C₁₋₈-alkyl-heterocyclyl, C₁₋₈-alkyl-aryl or C₁₋₈-alkyl-heteroaryl, wherein the alkyl is straight-chained or branched, saturated or unsaturated, and unsubstituted or mono- or poly-substituted, the cycloalkyl is saturated or unsaturated, unsubstituted or mono- or poly-substituted, the heterocyclyl is saturated or unsaturated, and unsubstituted or mono- or poly-substituted, the aryl is unsubstituted or mono- or poly-substituted, and the heteroaryl is unsubstituted or mono- or poly-substituted,
R⁴, R⁵, R⁶ and R⁷ each independently of the others represents hydrogen or C₁₋₈-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; F, Cl, Br, I, CN, NO₂, NH₂, C(═O)R⁹, CO₂H, CO₂R¹⁰ or OH, or
R⁴ and R⁵ or R⁵ and R⁶ or R⁶ and R⁷ represent a four-membered saturated or unsaturated hydrocarbon bridge having zero, 1, 2 or 3 hetero atoms selected from the group consisting of N, O and S, and the others of R⁴, R⁵, R⁶ and R⁷ represent hydrogen,
R⁸ represents C(═O)R⁹,
R⁹ represents C₁₋₈-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; heterocyclyl, which is saturated or unsaturated, and unsubstituted or mono- or poly-substituted; aryl, which is unsubstituted or mono- or poly-substituted; heteroaryl, which is unsubstituted or mono- or poly-substituted; C₁₋₈-alkyl-aryl or C₁₋₈-alkyl-heteroaryl, wherein the alkyl is straight-chained or branched, saturated or unsaturated, and unsubstituted or mono- or poly-substituted, the aryl is unsubstituted or mono- or poly-substituted, and the heteroaryl is unsubstituted or mono- or poly-substituted, and
R¹⁰ represents C₁₋₈-alkyl, which is straight-chained or branched, saturated or unsaturated, and unsubstituted or mono- or poly-substituted; C₃₋₈-cycloalkyl or CH₂—C₃₋₈-cycloalkyl, wherein the cycloalkyl is saturated or unsaturated, and unsubstituted or mono- or poly-substituted; aryl, which is unsubstituted or mono- or poly-substituted; C₁₋₈-alkyl-aryl, wherein alkyl is straight-chained or branched and is saturated or unsaturated and is unsubstituted or mono- or poly-substituted and aryl is unsubstituted or mono- or poly-substituted.

8. A phamarceutical according to claim 7, wherein in formula I
R¹ represents methyl, ethyl, n-propyl, 2-propyl, n-butyl, tert-butyl, n-hexyl, n-octyl, 1, 1,3,3-tetramethylbutyl; CH₂-aryl, which is unsubstituted or mono- or poly-substituted; CH₂CO₂—C₁₋₆-alkyl, wherein the alkyl is straight-chained or branched; CH₂PO(O—C₁₋₆-alkyl)₂, wherein the alkyl is straight-chained or branched; CH₂SiR²R¹³R¹⁴, CH₂CH₂-morpholin-4-yl; (CH₂)ₙ—NC, wherein n=2, 3, 4, 5 or 6; C₃₋₈-cycloalkyl, wherein the cycloalkyl is unsubstituted or mono- or poly-substituted; or phenyl, wherein the phenyl is unsubstituted or mono- or poly-substituted,
R² represents H or C(═O)—C₁₋₄-alkyl,
R³ represents methyl, ethyl, n-propyl, 2-propyl, n-butyl, tert-butyl, cyclopropyl, cyclopentyl or cyclohexyl, which are unsubstituted or mono- or poly-substituted; phenyl, which is unsubstituted or mono- or poly-substituted; 1-naphthyl or 2-naphthyl, wherein the naphthyl is unsubstituted or mono- or poly-substituted; 9-phenanthrenyl, pyrrol-2-yl, pyrrol-3-yl, pyridin-2-yl, pyridin-3-yl or pyridin-4-yl, wherein the pyrrolyl or pyridinyl are unsubstituted or mono- or poly-substituted; furan-2-yl or furan-3-yl, wherein the furanyl is unsubstituted or mono- or poly-substituted; thien-2-yl or thien-3-yl, wherein the thienyl is unsubstituted or mono- or poly-substituted; imidazol-2-yl, imidazol-4-yl, imidazol-5-yl, wherein the imidazolyl is unsubstituted or mono- or poly-substituted; thiazol-2-yl, thiazol-4-yl, thiazol-5-yl, wherein the thiazolyl is unsubstituted or mono- or poly-substituted; oxazol-2-yl, oxazol-4-yl, oxazol-5-yl, wherein the oxazolyl is unsubstituted or mono- or poly-substituted; isooxazol-3-yl, isooxazol-4-yl, isooxazol-5-yl, wherein the isooxazolyl is unsubstituted or mono- or poly-substituted; indol-2-yl, benzofuran-2-yl or benzofuran-3-yl,
R⁴, R⁵, R⁶ and R⁷ each independently of the others represents H, methyl, ethyl, n-propyl, 2-propyl, n-butyl, tert-butyl, CF₃, F, Cl, Br, I, CO₂H, CO₂-methyl, CO₂-ethyl, C(═O)CH₃ or NO₂, or R⁶ and R⁷ form the hydrocarbon bridge —CH═CH—CH═CH—,
R⁸ represents C(═O)CH₃ and
R¹², R¹³ and R¹⁴ each independently of the others represents C₁₋₆-alkyl, which is straight-chained or branched and is unsubstituted or mono- or poly-substituted; C₃₋₈-cycloalkyl or CH₂—C₃₋₈-cycloalkyl, wherein cycloalkyl is unsubstituted or mono- or poly-substituted; or phenyl, wherein phenyl is unsubstituted or mono- or poly-substituted.

9. A pharmaceutical composition according to claim 8, wherein in formula I
R¹ represents methyl, ethyl, n-propyl, 2-propyl, n-butyl, tert-butyl, n-hexyl, n-octyl, 1,1,3,3-tetramethylbutyl; CH₂-aryl, wherein the aryl is unsubstituted or mono- or poly-substituted; CH₂CO₂—C₁₋₆-alkyl, wherein the alkyl is straight-chained or branched; CH₂PO(O—C₁₋₆-alkyl)₂, wherein the alkyl is straight-chained or branched, CH₂SiR¹²R³R¹⁴, CH₂CH₂-morpholin-4-yl; (CH₂)ₙ—NC, wherein n=2, 3, 4, 5 or 6; C₃₋₈-cycloalkyl, wherein the cycloalkyl is unsubstituted or mono- or poly-substituted; or phenyl, which is unsubstituted or mono- or poly-substituted,
R² represents H or C(═O)—C₁₋₄-alkyl,
R³ represents methyl, ethyl, n-propyl, 2-propyl, n-butyl, tert-butyl, cyclopropyl, cyclopentyl or cyclohexyl, which, independently of one another, are unsubstituted or mono- or poly-substituted; phenyl, which is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy and dimethylamino; 1-naphthyl or 2-naphthyl, wherein the naphthyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy and dimethylamino; 9-phenanthrenyl, pyrrol-2-yl; pyridin-2-yl, pyridin-3-yl or pyridin-4-yl, wherein the pyridinyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; furan-2-yl or furan-3-yl, wherein the furanyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; thien-2-yl or thien-3-yl, wherein the thienyl is unsubstituted or monosubstituted or poly-substituted by identical or different substituents selected from the group consisting of methyl, ethyl, n-propyl, prop-2-yl, n-butyl, sec-butyl, tert-butyl, isobutyl, CF₃, OH, O-methyl, O-ethyl, F, Cl, Br, I, CN, NO₂, 4-chlorophenoxy, acetoxy, dimethylamino, carboxy, carboxymethyl, carboxyethyl, hydroxymethyl, chlorophenyl, nitrophenyl, [1,3]-dioxolan and methylsulfanyl; indol-2-yl, benzofuran-2-yl or benzofuran-3-yl,
R⁴, R⁵, R⁶ and R⁷ each independently of the others represents H, methyl, ethyl, n-propyl, 2-propyl, n-butyl, tert-butyl, CF₃, F, Cl, Br, I, CO₂H, CO₂-methyl, CO₂-ethyl, C(═O)CH₃ or NO₂, or
R⁶ and R⁷ form the hydrocarbon bridge —CH═CH—CH═CH—,
R⁸ represents C(═O)CH₃ and
R¹², R¹³ and R¹⁴ each independently of the others represents C₁₋₆-alkyl, which is straight-chained or branched, and unsubstituted or mono- or poly-substituted; C₃₋₈-cycloalkyl or CH₂—C₃₋₈-cycloalkyl, wherein the cycloalkyl is unsubstituted or mono- or poly-substituted; or phenyl, wherein the phenyl is unsubstituted or mono- or poly-substituted.

10. A pharmaceutical composition according to claim 9, wherein in formula I
R¹ represents methyl, n-butyl, 1,1,3,3-tetramethylbutyl, benzyl, 2-chlorobenzyl, 2-methoxybenzyl, CH₂CO₂CH₃, (CH₂)₆—NC, cyclopentyl, cyclohexyl, phenyl, 2,6-dimethylphenyl, 3-chlorophenyl or 3-chloro-4-fluorophenyl,
R² represents H or C(═O)CH₃,
R³ represents methyl, tert-butyl, cyclohexyl, phenyl, 2-methylphenyl, 3-methylphenyl, 4-methylphenyl, 2-trifluoromethylphenyl, 3-trifluoromethylphenyl, 4-trifluoromethylphenyl, 2-hydroxyphenyl, 2-methoxyphenyl, 3-hydroxyphenyl, 3-methoxyphenyl, 2-fluorophenyl, 3-fluorophenyl, 4-fluorophenyl, 2-chlorophenyl, 3-chlorophenyl, 4-chlorophenyl, 2-bromophenyl, 3-bromophenyl, 4-bromophenyl, 2-nitrophenyl, 3-nitrophenyl, 4-nitrophenyl, 3-(4-chlorophenoxy)-phenyl, 2,4-dimethylphenyl, 2,3-dimethoxyphenyl, 3,4-dimethoxyphenyl, 3-methoxy-4-acetoxyphenyl, 2,3-dichlorophenyl, 2,4-dichlorophenyl, 2-chloro-4-fluorophenyl, 2-chloro-6-fluorophenyl, 4-bromo-2-fluorophenyl, 3,4,5-trimethoxyphenyl, 1-naphthyl, 2-ethoxy-naphth-1-yl, 4-dimethylamino-napth-1-yl, 9-phenanthrenyl, pyrrol-2-yl, N-methylpyrrol-2-yl, pyridin-2-yl, pyridin-3-yl, pyridin-4-yl, furan-2-yl, furan-3-yl, 5-methyl-furan-2-yl, 4,5-dimethyl-furan-2-yl, 5-hydroxymethyl-furan-2-yl, 5-acetoxymethyl-furan-2-yl, 5-carboxy-furan-2-yl, 5-[1,3]-dioxolan-furan-2-yl, 3-bromo-furan-2-yl, 5-bromo-furan-2-yl, 5-nitro-furan-2-yl, 5-(2-nitrophenyl)-furan-2-yl, 5-(2-chlorophenyl)-furan-2-yl, 5-(3-chlorophenyl)-furan-2-yl, 5-(3-chlorophenyl)-furan-3-yl, 5-(4-chlorophenyl)-furan-2-yl, benzo[b]furan-2-yl, thien-2-yl, thien-3-yl, 5-methyl-thien-2-yl, 5-carboxy-thien-2-yl, 3-bromo-thien-2-yl, 5-chloro-thien-2-yl or 5-methylsulfanyl-thien-2-yl,
R⁴ represents H, CH₃, Cl, Br or CO₂H,
R⁵ represents H, CH₃, C₂H₅ or Cl,
R⁶ represents H, CH₃, Cl, Br or NO₂,
R⁷ represents H, CH₃ or n-C₃H₇ and
R⁸ represents C(═O)CH₃.

11. A pharmaceutical composition according to claim 10, wherein in formula I R⁴ and R⁶ represent H, R⁵ represents H, CH₃ or C₂H₅ and R⁷ represents H or CH₃.

12. A pharmaceutical composition according to claim 11, wherein the compound is selected from the group consisting of:
tert-butyl-(7-methyl-2-pyridin-3-yl-imidazo[1,2-a]pyridin-3-yl)-amine,
cyclohexyl-(5,7-dimethyl-2-pyridin-4-yl-imidazo[1,2-a]pyridin-3-yl)-amine,
(5,7-dimethyl-2-pyridin-4-yl-imidazo[1,2-a]pyridin-3-yl)-(1,1,3,3-tetramethyl-butyl)-amine,
{6-[5,7-dimethyl-2-(1H-pyrrol-2-yl)-imidazo[1,2-a]pyridin-3-ylamino]-hexyl}-methylidyne-ammonium,
tert-butyl-[2-(2,3-dimethoxy-phenyl)-5,7-dimethyl-imidazo[1,2-a]pyridin-3-yl]-amine,
[2-(3,4-dimethoxy-phenyl)-7-methyl-imidazo[1,2-a]pyridin-3-yl]-(1,1,3,3-tetramethyl-butyl)-amine,
cyclohexyl-(7-methyl-2-pyridin-4-yl-imidazo[1,2-a]pyridin-3-yl)-amine,
(2-furan-2-yl-5,7-dimethyl-imidazo[1,2-a]pyridin-3-yl)-(1,1,3,3-tetramethylbutyl)-amine,
(1,1,3,3-tetramethyl-butyl)-(2,5,7-trimethyl-imidazo[1,2-a]pyridin-3-yl)-amine,
cyclohexyl-(7-methyl-2-o-tolyl-imidazo[1,2-a]pyridin-3-yl)-amine,
cyclohexyl-(7-methyl-2-thiophen-2-yl-imidazo[1,2-a]pyridin-3-yl)-amine,
(5,7-dimethyl-2-pyridin-3-yl-imidazo[1,2-a]pyridin-3-yl)-(1,1,3,3-tetramethyl-butyl)-amine,
cyclohexyl-[7-methyl-2-(2-trifluoromethyl-phenyl)-imidazo[1,2-a]pyridin-3-yl]-amine,
tert-butyl-(2,5,7-trimethyl-imidazo[1,2-a]pyridin-3-yl)-amine,
(7-methyl-2-pyridin-3-yl-imidazo[1,2-a]pyridin-3-yl)-(1,1,3,3-tetramethylbutyl)-amine,
cyclohexyl-(7-methyl-2-naphthalen-1-yl-imidazo[1,2-a]pyridin-3-yl)-amine,
[2-(2-fluoro-phenyl)-7-methyl-imidazo[1,2-a]pyridin-3-yl]-(1,1,3,3-tetramethyl-butyl)-amine,
(2,7-dimethyl-imidazo[1,2-a]pyridin-3-ylamino)-acetic acid methyl ester,
methylidyne-[6-(7-methyl-2-pyridin-3-yl-imidazo[1,2-a]pyridin-3-ylamino)-hexyl]-ammonium,
3-(3-tert-butylamino-5,7-dimethyl-imidazo[1,2-a]pyridin-2-yl)-phenol,
cyclohexyl-[2-(2-fluoro-phenyl)-7-methyl-imidazo[1,2-a]pyridin-3-yl]-amine,
tert-butyl-(2-cyclohexyl-5,7-dimethyl-imidazo[1,2-a]pyridin-3-yl)-amine,
cyclohexyl-(7-methyl-2-pyridin-3-yl-imidazo[1,2-a]pyridin-3-yl)-amine,
3-(3-tert-butylamino-7-methyl-imidazo[1,2-a]pyridin-2-yl)-phenol,
tert-butyl-(2-furan-2-yl-5,7-dimethyl-imidazo[1,2-a]pyridin-3-yl)-amine,
cyclohexyl-(2-furan-2-yl-5,7-dimethyl-imidazo[1,2-a]pyridin-3-yl)-amine,
cyclohexyl-(5,7-dimethyl-2-pyridin-3-yl-imidazo[1,2-a]pyridin-3-yl)-amine,
(2-furan-2-yl-7-methyl-imidazo[1,2-a]pyridin-3-yl)-(1,1,3,3-tetramethylbutyl)-amine,
(7-methyl-2-pyridin-4-yl-imidazo[1,2-a]pyridin-3-yl)-(1,1,3,3-tetramethylbutyl)-amine,
butyl-(2-cyclohexyl-7-methyl-imidazo[1,2-a]pyridin-3-yl)-amine,
3-[5,7-dimethyl-3-(1,1,3,3-tetramethyl-butylamino)-imidazo[1,2-a]pyridin-2-yl]-phenol,
(2,6-dimethyl-phenyl)-(5,7-dimethyl-2-o-tolyl-imidazo[1,2-a]pyridin-3-yl)-amine,
tert-butyl-(7-methyl-2-naphthalen-1-yl-imidazo[1,2-a]pyridin-3-yl)-amine,
(2,6-dimethyl-phenyl)-[2-(2-fluoro-phenyl)-5,7-dimethyl-imidazo[1,2-a]-pyridin-3-yl]-amine,
cyclohexyl-(2,5,7-trimethyl-imidazo[1,2-a]pyridin-3-yl)-amine,
[5,7-dimethyl-2-(1H-pyrrol-2-yl)-imidazo[1,2-a]pyridin-3-yl]-(1,1,3,3-tetramethyl-butyl)-amine,
butyl-(2-cyclohexyl-5,7-dimethyl-imidazo[1,2-a]pyridin-3-yl)-amine,
(5,7-dimethyl-2-o-tolyl-imidazo[1,2-a]pyridin-3-yl)-(1,1,3,3-tetramethylbutyl)-amine,
[2-(2,3-dimethoxy-phenyl)-7-methyl-imidazo[1,2-a]pyridin-3-yl]-(2,6-dimethyl-phenyl)-amine,
(2,7-dimethyl-imidazo[1,2-a]pyridin-3-yl)-(1,1,3,3-tetramethyl-butyl)-amine,
[2-(5-[1,3]dioxolan-2-yl-furan-2-yl)-7-methyl-imidazo[1,2-a]pyridin-3-yl]-(1,1,3,3-tetramethyl-butyl)-amine,
[2-(3-bromo-thiophen-2-yl)-5,7-dimethyl-imidazo[1,2-a]pyridin-3-yl]-(2,6-dimethyl-phenyl)-amine,
(2,6-dimethyl-phenyl)-[2-(2-fluoro-phenyl)-7-methyl-imidazo[1,2-a]pyridin-3-yl]-amine,
(2-cyclohexyl-5,7-dimethyl-imidazo[1,2-a]pyridin-3-yl)-(1,1,3,3-tetramethylbutyl)-amine,
[6-(2-furan-2-yl-5,7-dimethyl-imidazo[1,2-a]pyridin-3-ylamino)-hexyl]-methylidyne-ammonium,
(7-methyl-2-o-tolyl-imidazo[1,2-a]pyridin-3-yl)-(1,1,3,3-tetramethyl-butyl)-amine,
[2-(2,3-dichloro-phenyl)-7-methyl-imidazo[1,2-a]pyridin-3-yl]-(2,6-dimethylphenyl)-amine,
[2-(2,3-dimethoxy-phenyl)-5,7-dimethyl-imidazo[1,2-a]pyridin-3-yl]-(2,6-dimethyl-phenyl)-amine,
butyl-[2-(2,3-dimethoxy-phenyl)-5,7-dimethyl-imidazo[1,2-a]pyridin-3-yl]-amine,
methylidyne-[6-(7-methyl-2-p-tolyl-imidazo[1,2-a]pyridin-3-ylamino)-hexyl]-ammonium,
tert-butyl-[5,7-dimethyl-2-(5-nitro-furan-2-yl)-imidazo[1,2-a]pyridin-3-yl]-amine,
acetic acid 5-(3-cyclohexylamino-5,7-dimethyl-imidazo[1,2-a]pyridin-2-yl)-furan-2-yl methyl ester,
[2-(2-methoxy-phenyl)-5,7-dimethyl-imidazo[1,2-a]pyridin-3-yl]-(1,1,3,3-tetramethyl-butyl)-amine,
[2-(3,4-dimethoxy-phenyl)-7-methyl-imidazo[1,2-a]pyridin-3-yl]-(2,6-dimethyl-phenyl)-amine,
3-(3-butylamino-7-methyl-imidazo[1,2-a]pyridin-2-yl)-phenol,
(2-benzofuran-2-yl-7-methyl-imidazo[1,2-a]pyridin-3-yl)-(1,1,3,3-tetramethyl-butyl)-amine,
(2-benzofuran-2-yl-5,7-dimethyl-imidazo[1,2-a]pyridin-3-yl)-(2,6-dimethylphenyl)-amine,
acetic acid 5-(3-cyclohexylamino-7-methyl-imidazo[1,2-a]pyridin-2-yl)-furan-2-yl methyl ester,
[6-(5,7-dimethyl-2-pyridin-3-yl-imidazo[1,2-a]pyridin-3-ylamino)-hexyl]-methylidyne-ammonium,
butyl-[2-(2-methoxy-phenyl)-7-methyl-imidazo[1,2-a]pyridin-3-yl]-amine,
{6-[2-(2-methoxy-phenyl)-5,7-dimethyl-imidazo[1,2-a]pyridin-3-ylamino]-hexyl}-methylidyne-ammonium,
{5-[5,7-dimethyl-3-(1,1,3,3-tetramethyl-butylamino)-imidazo[1,2-a]pyridin-2-yl]-furan-2-yl}-methanol,
(7-methyl-2-naphthalen-1-yl-imidazo[1,2-a]pyridin-3-yl)-(1,1,3,3-tetramethyl-butyl)-amine,
[5-(3-tert-butylamino-7-methyl-imidazo[1,2-a]pyridin-2-yl)-furan-2-yl]-methanol,
tert-butyl-[2-(5-[1,3]dioxolan-2-yl-furan-2-yl)-7-methyl-imidazo[1,2-a]-pyridin-3-yl]-amine,
(2-benzofuran-2-yl-5,7-dimethyl-imidazo[1,2-a]pyridin-3-yl)-(1,1,3,3-tetramethyl-butyl)-amine,
5-(3-tert-butylamino-5,7-dimethyl-imidazo[1,2-a]pyridin-2-yl)-furan-2-carboxylic acid,
tert-butyl-(2-furan-2-yl-7-methyl-imidazo[1,2-a]pyridin-3-yl)-amine,
cyclohexyl-(2,7-dimethyl-imidazo[1,2-a]pyridin-3-yl)-amine,
[2-(2,3-dichlorophenyl)-8-methyl-imidazo[1,2-a]pyridin-3-yl]-(1,1,3,3-tetramethyl-butyl)-amine,
(7-methyl-2-p-tolyl-imidazo[1,2-a]pyridin-3-yl)-(1,1,3,3-tetramethyl-butyl)-amine,
(2,6-dimethyl-phenyl)-[2-(2-methoxy-phenyl)-7-methyl-imidazo[1,2-a]-pyridin-3-yl]-amine,
3-(3-butylamino-5,7-dimethyl-imidazo[1,2-a]pyridin-2-yl)-phenol,
butyl-[2-(2,3-dimethoxy-phenyl)-7-methyl-imidazo[1,2-a]pyridin-3-yl]-amine,
{6-[5,7-dimethyl-2-(2-trifluoromethyl-phenyl)-imidazo[1,2-a]pyridin-3-ylamino]-hexyl}-methylidyne-ammonium,
tert-butyl-(7-methyl-2-pyridin-2-yl-imidazo[1,2-a]pyridin-3-yl)-amine,
cyclohexyl-(2-cyclohexyl-5,7-dimethyl-imidazo[1,2-a]pyridin-3-yl)-amine,
[2-(2,3-dichlorophenyl)-5,7-dimethyl-imidazo[1,2-a]pyridin-3-yl]-(2,6-dimethyl-phenyl)-amine,
(2,6-dimethyl-phenyl)-[2-(2-methoxy-phenyl)-5,7-dimethyl-imidazo[1,2-a]-pyridin-3-yl]-amine,
{2-[5-(2-chlorophenyl)-furan-2-yl]-7-methyl-imidazo[1,2-a]pyridin-3-yl}-(1,1,3,3-tetramethyl-butyl)-amine,
5-[7-methyl-3-(1,1,3,3-tetramethyl-butylamino)-imidazo[1,2-a]pyridin-2-yl]-furan-2-carboxylic acid,
cyclohexyl-[2-(2-methoxy-phenyl)-8-methyl-imidazo[1,2-a]pyridin-3-yl]-amine,
3-[7-methyl-3-(1,1,3,3-tetramethyl-butylamino)-imidazo[1,2-a]pyridin-2-yl]-phenol,
[2-(2,3-dichlorophenyl)-5,7-dimethyl-imidazo[1,2-a]pyridin-3-yl]-(1,1,3,3-tetramethyl-butyl)-amine,
[2-(2,4-dichlorophenyl)-7-methyl-imidazo[1,2-a]pyridin-3-yl]-(1,1,3,3-tetramethyl-butyl)-amine,
[2-(5-bromofuran-2-yl)-7-methyl-imidazo[1,2-a]pyridin-3-yl]-(1,1,3,3-tetramethyl-butyl)-amine,
5-(3-cyclohexylamino-5,7-dimethyl-imidazo[1,2-a]pyridin-2-yl)-furan-2-carboxylic acid,
[6-(2-cyclohexyl-5,7-dimethyl-imidazo[1,2-a]pyridin-3-ylamino)-hexyl]-methylidyne-ammonium,
[2-(2,4-dichlorophenyl)-5,7-dimethyl-imidazo[1,2-a]pyridin-3-yl]-(1,1,3,3-tetramethyl-butyl)-amine,
(2-benzofuran-2-yl-7-methyl-imidazo[1,2-a]pyridin-3-yl)-(2,6-dimethylphenyl)-amine,
5-(3-cyclohexylamino-7-methyl-imidazo[1,2-a]pyridin-2-yl)-furan-2-carboxylic acid,
{6-[2-(2-bromophenyl)-5,7-dimethyl-imidazo[1,2-a]pyridin-3-ylamino]-hexyl}-methylidyne-ammonium,
tert-butyl-(2-cyclohexyl-7-methyl-imidazo[1,2-a]pyridin-3-yl)-amine,
tert-butyl-(2,7-dimethyl-imidazo[1,2-a]pyridin-3-yl)-amine,
(5,7-dimethyl-2-p-tolyl-imidazo[1,2-a]pyridin-3-yl)-(1,1,3,3-tetramethylbutyl)-amine,
[2-(2,3-dichlorophenyl)-8-methyl-imidazo[1,2-a]pyridin-3-yl]-(2,6-dimethylphenyl)-amine,
methylidyne-[6-(7-methyl-2-o-tolyl-imidazo[1,2-a]pyridin-3-ylamino)-hexyl]-ammonium,
{2-[5-(3-chlorophenyl)-furan-2-yl]-7-methyl-imidazo[1,2-a]pyridin-3-yl}-(1,1,3,3-tetramethyl-butyl)-amine,
cyclohexyl-[7-methyl-2-(5-nitro-furan-2-yl)-imidazo[1,2-a]pyridin-3-yl]-amine,
[2-(2-bromophenyl)-7-methyl-imidazo[1,2-a]pyridin-3-yl]-cyclohexyl-amine,
[2-(2-methoxy-phenyl)-7-methyl-imidazo[1,2-a]pyridin-3-yl]-(1,1,3,3-tetramethyl-butyl)-amine,
{5-[7-methyl-3-(1,1,3,3-tetramethyl-butylamino)-imidazo[1,2-a]pyridin-2-yl]-furan-2-yl}-methanol,
(6-{2-[5-(2-chlorophenyl)-furan-2-yl]-5-methyl-imidazo[1,2-a]pyridin-3-ylamino}-hexyl)-methylidyne-ammonium,
cyclohexyl-[5,7-dimethyl-2-(5-nitro-furan-2-yl)-imidazo[1,2-a]pyridin-3-yl]-amine,
cyclohexyl-[2-(4,5-dimethyl-furan-2-yl)-7-methyl-imidazo[1,2-a]pyridin-3-yl]-amine,
[6-(5,7-dimethyl-2-o-tolyl-imidazo[1,2-a]pyridin-3-ylamino)-hexyl]-methylidyne-ammonium,
methylidyne-[6-(7-methyl-2-pyridin-2-yl-imidazo[1,2-a]pyridin-3-ylamino)-hexyl]-ammonium,
[2-(2,3-dimethoxy-phenyl)-5,7-dimethyl-imidazo[1,2-a]pyridin-3-yl]-(1,1,3,3-tetramethyl-butyl)-amine,
{6-[2-(3,4-dimethoxy-phenyl)-7-methyl-imidazo[1,2-a]pyridin-3-ylamino]-hexyl}-methylidyne-ammonium,
5-(3-tert-butylamino-7-methyl-imidazo[1,2-a]pyridin-2-yl)-thiophene-2-carboxylic acid,
cyclohexyl-(8-methyl-2-pyridin-4-yl-imidazo[1,2-a]pyridin-3-yl)-amine,
[2-(2,3-dichloro-phenyl)-6-methyl-imidazo[1,2-a]pyridin-3-yl]-(1,1,3,3-tetramethyl-butyl)-amine,
5-(3-butylamino-imidazo[1,2-a]pyrazin-2-yl)-thiophene-2-carboxylic acid,
cyclohexyl-(5,7-dimethyl-2-pyridin-2-yl-imidazo[1,2-a]pyridin-3-yl)-amine,
(2-benzofuran-2-yl-8-methyl-imidazo[1,2-a]pyridin-3-yl)-(1,1,3,3-tetramethyl-butyl)-amine,
{6-[2-(2-fluoro-phenyl)-5,7-dimethyl-imidazo[1,2-a]pyridin-3-ylamino]-hexyl}-methylidyne-ammonium,
[2-(2,3-dimethoxy-phenyl)-8-methyl-imidazo[1,2-a]pyridin-3-yl]-(1,1,3,3-tetramethyl-butyl)-amine,
methylidyne-[6-(7-methyl-2-phenanthren-9-yl-imidazo[1,2-a]pyridin-3-ylamino)-hexyl]-ammonium,
5-(3-tert-butylamino-imidazo[1,2-a]pyrazin-2-yl)-thiophene-2-carboxylic acid,
tert-butyl-(8-methyl-2-pyridin-2-yl-imidazo[1,2-a]pyridin-3-yl)-amine,
cyclohexyl-(2-furan-2-yl-8-methyl-imidazo[1,2-a]pyridin-3-yl)-amine,
methylidyne-[6-(7-methyl-2-naphthalen-1-yl-imidazo[1,2-a]pyridin-3-ylamino)-hexyl]-ammonium,
tert-butyl-(2-cyclohexyl-8-methyl-imidazo[1,2-a]pyridin-3-yl)-amine,
(6-methyl-2-pyridin-4-yl-imidazo[1,2-a]pyridin-3-yl)-(1,1,3,3-tetramethylbutyl)-amine,
tert-butyl-(6-methyl-2-pyridin-3-yl-imidazo[1,2-a]pyridin-3-yl)-amine,
(7-methyl-2-pyridin-3-yl-imidazo[1,2-a]pyrimidin-3-yl)-(1,1,3,3-tetramethylbutyl)-amine,
5-(3-tert-butylamino-6-methyl-imidazo[1,2-a]pyridin-2-yl)-thiophene-2-carboxylic acid,
[6-(5,7-dimethyl-2-naphthalen-1-yl-imidazo[1,2-a]pyridin-3-ylamino)-hexyl]-methylidyne-ammonium,
3-[3-(2,6-dimethyl-phenylamino)-5,7-dimethyl-imidazo[1,2-a]pyridin-2-yl]-phenol,
(2,6-dimethyl-phenyl)-(8-methyl-2-o-tolyl-imidazo[1,2-a]pyridin-3-yl)-amine,
{6-[2-(3-hydroxy-phenyl)-8-methyl-imidazo[1,2-a]pyridin-3-ylamino]-hexyl}-methylidyne-ammonium,
{5-[3-(2,6-dimethyl-phenylamino)-7-methyl-imidazo[1,2-a]pyrimidin-2-yl]-furan-2-yl}-methanol,
(8-methyl-2-p-tolyl-imidazo[1,2-a]pyridin-3-yl)-(1,1,3,3-tetramethyl-butyl)-amine,
[2-(2,4-dichlorophenyl)-6-methyl-imidazo[1,2-a]pyridin-3-yl]-(2,6-dimethylphenyl)-amine,
butyl-[2-(2,4-dichloro-phenyl)-6-methyl-imidazo[1,2-a]pyridin-3-yl]-amine,
butyl-[2-(4-dimethylamino-naphthalen-1-yl)-imidazo[1,2-a]pyrazin-3-yl]-amine,
{6-[2-(2-bromo-phenyl)-7-methyl-imidazo[1,2-a]pyridin-3-ylamino]-hexyl}-methylidyne-ammonium,
butyl-[2-(2-methoxy-phenyl)-6-methyl-imidazo[1,2-a]pyridin-3-yl]-amine,
(2-cyclohexyl-8-methyl-imidazo[1,2-a]pyridin-3-yl)-(1,1,3,3-tetramethylbutyl)-amine,
cyclohexyl-(7-methyl-2-pyridin-2-yl-imidazo[1,2-a]pyridin-3-yl)-amine,
cyclohexyl-(2-furan-2-yl-7-methyl-imidazo[1,2-a]pyridin-3-yl)-amine,
(2-cyclohexyl-5,7-dimethyl-imidazo[1,2-a]pyridin-3-ylamino)-acetic acid methyl ester,
N-(2-furan-2-yl-5,7-dimethyl-imidazo[1,2-a]pyridin-3-yl)-N-(1,1,3,3-tetramethyl-butyl)-acetamide,
N-tert-butyl-N-(7-methyl-2-pyridin-3-yl-imidazo[1,2-a]pyridin-3-yl)-acetamide,
N-tert-butyl-N-(2-furan-2-yl-5,7-dimethyl-imidazo[1,2-a]pyridin-3-yl)-acetamide,
N-(5,7-dimethyl-2-pyridin-4-yl-imidazo[1,2-a]pyridin-3-yl)-N-(1,1,3,3-tetramethyl-butyl)-acetamide,
N-(5,7-dimethyl-2-pyridin-3-yl-imidazo[1,2-a]pyridin-3-yl)-N-(1,1,3,3-tetramethyl-butyl)-acetamide,
N-(2,6-dimethyl-phenyl)-N-(5,7-dimethyl-2-o-tolyl-imidazo[1,2-a]pyridin-3-yl)-acetamide,
N-(2-furan-2-yl-7-methyl-imidazo[1,2-a]pyridin-3-yl)-N-(1,1,3,3-tetramethylbutyl)-acetamide,
N-(1,1,3,3-tetramethyl-butyl)-N-(2,5,7-trimethyl-imidazo[1,2-a]pyridin-3-yl)-acetamide,
N-cyclohexyl-N-(7-methyl-2-thiophen-2-yl-imidazo[1,2-a]pyridin-3-yl)-acetamide,
N-tert-butyl-N-(2,5,7-trimethyl-imidazo[1,2-a]pyridin-3-yl)-acetamide,
5-[3-(acetyl-tert-butyl-amino)-imidazo[1,2-a]pyrazin-2-yl]-thiophene-2-carboxylic acid,
5-{3-[acetyl-(1,1,3,3-tetramethyl-butyl)-amino]-5,7-dimethyl-imidazo[1,2-a]-pyridin-2-yl}-furan-2-carboxylic acid,
N-[2-(5-hydroxymethyl-furan-2-yl)-5,7-dimethyl-imidazo[1,2-a]pyridin-3-yl]-N-(1,1,3,3-tetramethyl-butyl)-acetamide,
N-[2-(3-bromo-thiophen-2-yl)-5,7-dimethyl-imidazo[1,2-a]pyridin-3-yl]-N-(2,6-dimethyl-phenyl)-acetamide,
N-tert-butyl-N-(2-cyclohexyl-5,7-dimethyl-imidazo[1,2-a]pyridin-3-yl)-acetamide,
acetic acid 5-[3-(acetyl-cyclohexyl-amino)-5,7-dimethyl-imidazo[1,2-a]-pyridin-2-yl]-furan-2-yl methyl ester,
{6-[acetyl-(2-furan-2-yl-5,7-dimethyl-imidazo[1,2-a]pyridin-3-yl)-amino]-hexyl}-methylidyne-ammonium,
N-[2-(2,3-dichloro-phenyl)-7-methyl-imidazo[1,2-a]pyridin-3-yl]-N-(2,6-dimethyl-phenyl)-acetamide,
N-[2-(3-bromo-thiophen-2-yl)-imidazo[1,2-a]pyridin-3-yl]-N-cyclohexyl-acetamide,
N-(5,7-dimethyl-2-p-tolyl-imidazo[1,2-a]pyridin-3-yl)-N-(1,1,3,3-tetramethyl-butyl)-acetamide,
N-cyclohexyl-N-(7-methyl-2-pyridin-4-yl-imidazo[1,2-a]pyridin-3-yl)acetamide,
N-cyclohexyl-N-[7-methyl-2-(2-trifluoromethyl-phenyl)-imidazo[1,2-a]-pyridin-3-yl]-acetamide,
N-(6,8-dibromo-2-furan-2-yl-imidazo[1,2-a]pyridin-3-yl)-N-(1,1,3,3-tetramethyl-butyl)-acetamide,
N-(7-methyl-2-pyridin-4-yl-imidazo[1,2-a]pyridin-3-yl)-N-(1,1,3,3-tetramethyl-butyl)-acetamide,
acetic acid 5-[3-(acetyl-cyclohexyl-amino)-7-methyl-imidazo[1,2-a]pyridin-2-yl]-furan-2-yl methyl ester,
N-(7-methyl-2-pyridin-3-yl-imidazo[1,2-a]pyridin-3-yl)-N-(1,1,3,3-tetramethyl-butyl)-acetamide,
N-[2-(2,3-dichloro-phenyl)-5,7-dimethyl-imidazo[1,2-a]pyridin-3-yl]-N-(2,6-dimethyl-phenyl)-acetamide,
N-cyclohexyl-N-[5,7-dimethyl-2-(5-methyl-furan-2-yl)-imidazo[1,2-a]-pyridin-3-yl]-acetamide,
N-butyl-N-[2-(2,3-dimethoxy-phenyl)-5,7-dimethyl-imidazo[1,2-a]pyridin-3-yl]-acetamide,
N-[2-(2-methoxy-phenyl)-5,7-dimethyl-imidazo[1,2-a]pyridin-3-yl]-N-(1,1,3,3-tetramethyl-butyl)-acetamide,
N-cyclohexyl-N-[5,7-dimethyl-2-(5-nitro-furan-2-yl)-imidazo[1,2-a]pyridin-3-yl]-acetamide,
[acetyl-(2,7-dimethyl-imidazo[1,2-a]pyridin-3-yl)-amino]-acetic acid methyl ester,
N-cyclohexyl-N-(2,5,7-trimethyl-imidazo[1,2-a]pyridin-3-yl)-acetamide,
5-{3-[acetyl-(1,1,3,3-tetramethyl-butyl)-amino]-imidazo[1,2-a]pyridin-2-yl}-thiophene-2-carboxylic acid,
N-[2-(2,4-dichloro-phenyl)-5,7-dimethyl-imidazo[1,2-a]pyridin-3-yl]-N-(1,1,3,3-tetramethyl-butyl)-acetamide,
N-cyclohexyl-N-[7-methyl-2-(5-nitro-furan-2-yl)-imidazo[1,2-a]pyridin-3-yl]-acetamide,
N-(2-tert-butyl-5,7-dimethyl-imidazo[1,2-a]pyridin-3-yl)-N-(2,6-dimethylphenyl)-acetamide,
N-(2,6-dimethyl-phenyl)-N-[2-(2-methoxy-phenyl)-5,7-dimethyl-imidazo[1,2-a]pyridin-3-yl]-acetamide,
N-[2-(3-hydroxy-phenyl)-5,7-dimethyl-imidazo[1,2-a]pyridin-3-yl]-N-(1,1,3,3-tetramethyl-butyl)-acetamide,
N-(2,6-dimethyl-phenyl)-N-[2-(2-fluoro-phenyl)-5,7-dimethyl-imidazo[1,2-a]pyridin-3-yl]-acetamide,
5-[3-(acetyl-tert-butyl-amino)-5-methyl-imidazo[1,2-a]pyridin-2-yl]-thiophene-2-carboxylic acid,
N-(2,6-dimethyl-phenyl)-N-[2-(2-methoxy-phenyl)-7-methyl-imidazo[1,2-a]-pyridin-3-yl]-acetamide,
N-(7-methyl-2-p-tolyl-imidazo[1,2-a]pyridin-3-yl)-N-(1,1,3,3-tetramethylbutyl)-acetamide,
5-{3-[acetyl-(1,1,3,3-tetramethyl-butyl)-amino]-7-methyl-imidazo[1,2-a]-pyridin-2-yl}-furan-2-carboxylic acid,
N-cyclohexyl-N-(7-methyl-2-pyridin-3-yl-imidazo[1,2-a]pyridin-3-yl)-acetamide,
N-[2-(5-[1,3]dioxolan-2-yl-furan-2-yl)-5,7-dimethyl-imidazo[1,2-a]pyridin-3-yl]-N-(1,1,3,3-tetramethyl-butyl)-acetamide,
N-(2-benzofuran-2-yl-5,7-dimethyl-imidazo[1,2-a]pyridin-3-yl)-N-(2,6-dimethyl-phenyl)-acetamide,
N-tert-butyl-N-(2-furan-2-yl-7-methyl-imidazo[1,2-a]pyridin-3-yl)-acetamide,
N-tert-butyl-N-(7-methyl-2-pyridin-2-yl-imidazo[1,2-a]pyridin-3-yl)-acetamide,
N-cyclohexyl-N-(5,7-dimethyl-2-pyridin-3-yl-imidazo[1,2-a]pyridin-3-yl)-acetamide,
N-[2-(2,3-dichloro-phenyl)-5,7-dimethyl-imidazo[1,2-a]pyridin-3-yl]-N-(1,1,3,3-tetramethyl-butyl)-acetamide,
N-[2-(2,3-dimethoxy-phenyl)-5,7-dimethyl-imidazo[1,2-a]pyridin-3-yl]-N-(1,1,3,3-tetramethyl-butyl)-acetamide,
N-{2-[3-(4-chloro-phenoxy)-phenyl]-imidazo[1,2-a]pyridin-3-yl}-N-(2,6-dimethyl-phenyl)-acetamide,
N-[2-(5-[1,3]dioxolan-2-yl-furan-2-yl)-7-methyl-imidazo[1,2-a]pyridin-3-yl]-N-(1,1,3,3-tetramethyl-butyl)-acetamide,
5-[3-(acetyl-cyclohexyl-amino)-5,7-dimethyl-imidazo[1,2-a]pyridin-2-yl]-furan-2-carboxylic acid,
N-tert-butyl-N-[7-methyl-2-(5-nitro-furan-2-yl)-imidazo[1,2-a]pyridin-3-yl]-acetamide,
N-[2-(2-methoxy-phenyl)-7-methyl-imidazo[1,2-a]pyridin-3-yl]-N-(1,1,3,3-tetramethyl-butyl)-acetamide,
N-[2-(5-methyl-furan-2-yl)-imidazo[1,2-a]pyridin-3-yl]-N-(1,1,3,3-tetramethyl-butyl)-acetamide,
5-[3-(acetyl-tert-butyl-amino)-7-methyl-imidazo[1,2-a]pyridin-2-yl]-furan-2-carboxylic acid,
N-[2-(4,5-dimethyl-furan-2-yl)-imidazo[1,2-a]pyridin-3-yl]-N-(1,1,3,3-tetramethyl-butyl)-acetamide,
N-cyclohexyl-N-(2-furan-2-yl-5,7-dimethyl-imidazo[1,2-a]pyridin-3-yl)-acetamide,
N-tert-butyl-N-(7-methyl-2-naphthalen-1-yl-imidazo[1,2-a]pyridin-3-yl)-acetamide,
5-{3-[acetyl-(1,1,3,3-tetramethyl-butyl)-amino]-imidazo[1,2-a]pyrazin-2-yl}-thiophene-2-carboxylic acid,
N-butyl-N-(2-o-tolyl-imidazo[1,2-a]pyrimidin-3-yl)acetamide,
N-[2-(3,4-dimethoxy-phenyl)-7-methyl-imidazo[1,2-a]pyridin-3-yl]-N-(2,6-dimethyl-phenyl)-acetamide,
N-tert-butyl-N-(7-methyl-2-phenanthren-9-yl-imidazo[1,2-a]pyridin-3-yl)-acetamide,
N-(2,6-dimethyl-phenyl)-N-[2-(2-fluoro-phenyl)-7-methyl-imidazo[1,2-a]-pyridin-3-yl]-acetamide,
N-[2-(2-methoxy-phenyl)-8-methyl-imidazo[1,2-a]pyridin-3-yl]-N-(1,1,3,3-tetramethyl-butyl)-acetamide,
N-(2,6-dimethyl-phenyl)-N-[2-(3-hydroxy-phenyl)-5,7-dimethyl-imidazo[1,2-a]-pyridin-3-yl]-acetamide,
N-(2-tert-butyl-7-methyl-imidazo[1,2-a]pyridin-3-yl)-N-(2,6-dimethylphenyl)-acetamide,
acetic acid 4-{3-[acetyl-(2,6-dimethyl-phenyl)-amino]-6-bromo-8-methyl-imidazo[1,2-a]pyridin-2-yl}-2-methoxy-phenyl ester,
N-tert-butyl-N-[2-(5-[1,3]dioxolan-2-yl-furan-2-yl)-7-methyl-imidazo[1,2-a]-pyridin-3-yl]-acetamide,
[6-(acetyl-{7-methyl-2-[5-(2-nitro-phenyl)-furan-2-yl]-imidazo[1,2-a]pyridin-3-yl }-amino)-hexyl]-methylidyne-ammonium,
N-(2-benzofuran-2-yl-7-methyl-imidazo[1,2-a]pyridin-3-yl)-N-(1,1,3,3-tetramethyl-butyl)-acetamide,
N-(2-benzofuran-2-yl-5,7-dimethyl-imidazo[1,2-a]pyridin-3-yl)-N-(1,1,3,3-tetramethyl-butyl)-acetamide,
5-[3-(acetyl-tert-butyl-amino)-imidazo[1,2-a]pyridin-2-yl]-thiophene-2-carboxylic acid,
N-(2-cyclohexyl-5,7-dimethyl-imidazo[1,2-a]pyridin-3-yl)-N-(1,1,3,3-tetramethyl-butyl)-acetamide,
N-tert-butyl-N-[2-(5-methyl-furan-2-yl)-imidazo[1,2-a]pyridin-3-yl]-acetamide,
N-tert-butyl-N-[2-(5-methylsulfanyl-thiophen-2-yl)-imidazo[1,2-a]pyrimidin-3-yl]-acetamide,
N-[2-(4,5-dimethyl-furan-2-yl)-5-methyl-imidazo[1,2-a]pyridin-3-yl]-N-(1,1,3,3-tetramethyl-butyl)-acetamide,
N-butyl-N-[2-(2,4-dichloro-phenyl)-imidazo[1,2-a]pyrimidin-3-yl]-acetamide,
N-[2-(3-bromo-thiophen-2-yl)-6-methyl-imidazo[1,2-a]pyridin-3-yl]-N-cyclohexyl-acetamide,
5-{3-[acetyl-(2,6-dimethyl-phenyl)-amino]-6-methyl-imidazo[1,2-a]pyridin-2-yl}-thiophene-2-carboxylic acid,
N-butyl-N-[2-(2,3-dimethoxy-phenyl)-7-methyl-imidazo[1,2-a]pyridin-3-yl]-acetamide,
N-tert-butyl-N-[2-(2,3-dichloro-phenyl)-imidazo[1,2-a]pyridin-3-yl]-acetamide,
N-(2-furan-2-yl-5-propyl-imidazo[1,2-a]pyridin-3-yl)-N-(1,1,3,3-tetramethylbutyl)-acetamide,
5-[3-(acetyl-cyclohexyl-amino)-imidazo[1,2-a]pyridin-2-yl]-thiophene-2-carboxylic acid,
5-{3-[acetyl-(1,1,3,3-tetramethyl-butyl)-amino]-8-methyl-imidazo[1,2-a]-pyridin-2-yl}-furan-2-carboxylic acid,
3-(acetyl-butyl-amino)-2-pyridin-2-yl-imidazo[1,2-a]pyridine-8-carboxylic acid,
{6-[acetyl-(5,7-dimethyl-2-pyridin-3-yl-imidazo[1,2-a]pyridin-3-yl)-amino]-hexyl}-methylidyne-ammonium,
N-tert-butyl-N-[2-(5-methylsulfanyl-thiophen-2-yl)-imidazo[1,2-a]pyrazin-3-yl]-acetamide,
5-[3-(acetyl-cyclohexyl-amino)-5-methyl-imidazo[1,2-a]pyridin-2-yl]-thiophene-2-carboxylic acid,
N-[2-(5-methylsulfanyl-thiophen-2-yl)-imidazo[1,2-a]pyrazin-3-yl]-N-(1,1,3,3-tetramethyl-butyl)-acetamide,
N-[2-(2,3-dichloro-phenyl)-5-methyl-imidazo[1,2-a]pyridin-3-yl]-N-(2,6-dimethyl-phenyl)-acetamide,
N-butyl-N-[2-(2-methoxy-phenyl)-imidazo[1,2-a]pyridin-3-yl]-acetamide,
(6-{acetyl-[2-(2-methoxy-phenyl)-6-nitro-imidazo[1,2-a]pyridin-3-yl]-amino}-hexyl)-methylidyne-ammonium,
N-(2-benzofuran-2-yl-7-methyl-imidazo[1,2-a]pyridin-3-yl)-N-(2,6-dimethylphenyl)-acetamide,
(6-{acetyl-[2-(2-methoxy-phenyl)-5,7-dimethyl-imidazo[1,2-a]pyridin-3-yl]-amino}-hexyl)-methylidyne-ammonium,
{6-[acetyl-(7-methyl-2-pyridin-3-yl-imidazo[1,2-a]pyridin-3-yl)-amino]-hexyl}-methylidyne-ammonium,
N-(6-methyl-2-p-tolyl-imidazo[1,2-a]pyridin-3-yl)-N-(1,1,3,3-tetramethylbutyl)-acetamide,
acetic acid 5-{3-[acetyl-(2,6-dimethyl-phenyl)-amino]-5,7-dimethyl-imidazo[1,2-a]pyridin-2-yl}-furan-2-yl methyl ester,
{acetyl-[2-(3-hydroxy-phenyl)-6-methyl-imidazo[1,2-a]pyridin-3-yl]-amino}-acetic acid methyl ester,
N-tert-butyl-N-[2-(2-trifluoromethyl-phenyl)-imidazo[1,2-a]pyridin-3-yl]-acetamide,
N-butyl-N-[2-(2-chloro-4-fluoro-phenyl)-imidazo[1,2-a]pyridin-3-yl]-acetamide,
N-[2-(2,4-dichloro-phenyl)-imidazo[1,2-a]pyridin-3-yl]-N-(2,6-dimethylphenyl)-acetamide,
5-{3-[acetyl-(1,1,3,3-tetramethyl-butyl)-amino]-7-methyl-imidazo[1,2-a]-pyrimidin-2-yl}-furan-2-carboxylic acid,
acetic acid 5-{3-[acetyl-(1,1,3,3-tetramethyl-butyl)-amino]-imidazo[1,2-a]-pyrimidin-2-yl}-furan-2-yl methyl ester,
N-(2,7-dimethyl-imidazo[1,2-a]pyridin-3-yl)-N-(1,1,3,3-tetramethyl-butyl)-acetamide,
acetic acid 4-[3-(acetyl-cyclohexyl-amino)-5-amino-7-chloro-imidazo[1,2-a]-pyrimidin-2-yl]-2-methoxy-phenyl ester,
acetic acid 4-[3-(acetyl-cyclohexyl-amino)-5,7-dimethyl-imidazo[1,2-a]-pyrimidin-2-yl]-2-methoxy-phenyl ester,
N-[6-bromo-2-(2-chloro-6-fluoro-phenyl)-8-methyl-imidazo[1,2-a]pyridin-3-yl]-N-cyclohexyl-acetamide,
N-[2-(2-chloro-6-fluoro-phenyl)-5-methyl-imidazo[1,2-a]pyridin-3-yl]-N-cyclohexyl-acetamide,
N-butyl-N-[2-(2,3-dichloro-phenyl)-imidazo[1,2-a]pyrimidin-3-yl]-acetamide,
N-[2-(5-chloro-thiophen-2-yl)-imidazo[1,2-a]pyrimidin-3-yl]-N-(1,1,3,3-tetramethyl-butyl)-acetamide,
[acetyl-(2-cyclohexyl-7-methyl-imidazo[1,2-a]pyridin-3-yl)-amino]-acetic acid methyl ester,
N-tert-butyl-N-[2-(2-chloro-6-fluoro-phenyl)-imidazo[1,2-a]pyridin-3-yl]-acetamide,
N-cyclohexyl-N-(5,7-dimethyl-2-pyridin-2-yl-imidazo[1,2-a]pyridin-3-yl)-acetamide,
acetic acid 5-[3-(acetyl-cyclohexyl-amino)-5-methyl-imidazo[1,2-a]pyridin-2-yl]-furan-2-yl methyl ester,
N-(2,6-dimethyl-phenyl)-N-[6-methyl-2-(2-trifluoromethyl-phenyl)-imidazo[1,2-a]pyridin-3-yl]-acetamide,
N-cyclohexyl-N-(2-furan-2-yl-7-methyl-imidazo[1,2-a]pyridin-3-yl)-acetamide,
N-cyclohexyl-N-(7-methyl-2-pyridin-2-yl-imidazo[1,2-a]pyridin-3-yl)-acetamide,
N-cyclohexyl-N-[2-(5-[1,3]dioxolan-2-yl-furan-2-yl)-5-methyl-imidazo[1,2-a]pyridin-3-yl]-acetamide,
N-tert-butyl-N-(5-propyl-2-pyridin-3-yl-imidazo[1,2-a]pyridin-3-yl)-acetamide,
N-tert-butyl-N-[2-(5-methyl-thiophen-2-yl)-imidazo[1,2-a]pyrimidin-3-yl]-acetamide,
3-[acetyl-(1,1,3,3-tetramethyl-butyl)-amino]-2-furan-2-yl-imidazo[1,2-a]-pyridine-8-carboxylic acid,
N-tert-butyl-N-[2-(4,5-dimethyl-furan-2-yl)-6-methyl-imidazo[1,2-a]pyridin-3-yl]-acetamide,
N-{2-[3-(4-chloro-phenoxy)-phenyl]-imidazo[1,2-a]pyridin-3-yl}-N-cyclohexyl-acetamide,
acetic acid 4-[3-(acetyl-cyclohexyl-amino)-imidazo[1,2-a]pyrimidin-2-yl]-2-methoxy-phenyl ester,
N-[2-(5-bromo-furan-2-yl)-8-methyl-imidazo[1,2-a]pyridin-3-yl]-N-(1,1,3,3-tetramethyl-butyl)-acetamide,
N-(2,6-dimethyl-phenyl)-N-[2-(3-hydroxy-phenyl)-5,7-dimethyl-imidazo[1,2-a]pyrimidin-3-yl]-acetamide,
N-cyclohexyl-N-[2-(2,3-dichloro-phenyl)-imidazo[1,2-a]pyridin-3-yl]-acetamide,
N-cyclohexyl-N-[2-(2,4-dichloro-phenyl)-5-methyl-imidazo[1,2-a]pyridin-3-yl]-acetamide,
N-cyclohexyl-N-[2-(2,4-dichloro-phenyl)-imidazo[1,2-a]pyrimidin-3-yl]-acetamide,
[acetyl-(2-o-tolyl-imidazo[1,2-a]pyrazin-3-yl)-amino]-acetic acid methyl ester,
N-tert-butyl-N-(6,8-dichloro-2-thiophen-2-yl-imidazo[1,2-a]pyridin-3-yl)-acetamide,
N-tert-butyl-N-(5-propyl-2-thiophen-2-yl-imidazo[1,2-a]pyridin-3-yl)-acetamide,
{6-[acetyl-(7-methyl-2-p-tolyl-imidazo[1,2-a]pyridin-3-yl)-amino]-hexyl}-methylidyne-ammonium,
N-butyl-N-(6-methyl-2-p-tolyl-imidazo[1,2-a]pyridin-3-yl)-acetamide,
(6-{acetyl-[2-(2-methoxy-phenyl)-7-methyl-imidazo[1,2-a]pyridin-3-yl]-amino}-hexyl)-methylidyne-ammonium,
5-{3-[acetyl-(1,1,3,3-tetramethyl-butyl)-amino]-6-methyl-imidazo[1,2-a]-pyridin-2-yl}-furan-2-carboxylic acid,
N-butyl-N-[2-(3,4,5-trimethoxy-phenyl)-imidazo[1,2-a]pyrazin-3-yl]-acetamide,
N-butyl-N-[2-(3-hydroxy-phenyl)-imidazo[1,2-a]pyrimidin-3-yl]-acetamide,
[acetyl-(2-o-tolyl-imidazo[1,2-a]pyrimidin-3-yl)-amino]-acetic acid methyl ester,
N-(2-benzofuran-2-yl-8-methyl-imidazo[1,2-a]pyridin-3-yl)-N-(1,1,3,3-tetramethyl-butyl)-acetamide,
N-butyl-N-(7-methyl-2-p-tolyl-imidazo[1,2-a]pyrimidin-3-yl)-acetamide,
N-tert-butyl-N-(6,8-dibromo-2-methyl-imidazo[1,2-a]pyridin-3-yl)-acetamide,
{6-[acetyl-(5,7-dimethyl-2-pyridin-2-yl-imidazo[1,2-a]pyridin-3-yl)-amino]-hexyl}-methylidyne-ammonium,
N-tert-butyl-N-[2-(2-ethoxy-naphthalen-1-yl)-7-methyl-imidazo[1,2-a]-pyridin-3-yl]-acetamide,
N-tert-butyl-N-[2-(2-chloro-4-fluoro-phenyl)-imidazo[1,2-a]pyridin-3-yl]-acetamide,
cyclohexyl-[7-methyl-2-(2-trifluoromethyl-phenyl)-imidazo[1,2-a]pyridin-3-yl]-amine hydrochloride,
tert-butyl-(2-furan-2-yl-5,7-dimethyl-imidazo[1,2-a]pyridin-3-yl)-amine hydrochloride,
tert-butyl-(7-methyl-2-phenyl-imidazo[1,2-a]pyridin-3-yl)-amine hydrochloride,
cyclohexyl-(5,7-dimethyl-2-pyridin-4-yl-imidazo[1,2-a]pyridin-3-yl)-amine hydrochloride,
(2-furan-2-yl-5,7-dimethyl-imidazo[1,2-a]pyridin-3-yl)-(1,1,3,3-tetramethylbutyl)-amine hydrochloride,
tert-butyl-(2,5,7-trimethyl-imidazo[1,2-a]pyridin-3-yl)-amine hydrochloride,
[2-(2-fluorophenyl)-7-methyl-imidazo[1,2-a]pyridin-3-yl]-(1,1,3,3-tetramethyl-butyl)-amine hydrochloride,
cyclohexyl-(7-methyl-2-phenyl-imidazo[1,2-a]pyridin-3-yl)-amine hydrochloride,
(2-furan-2-yl-7-methyl-imidazo[1,2-a]pyrimidin-3-yl)-(1,1,3,3-tetramethylbutyl)-amine hydrochloride,
tert-butyl-[2-(4-nitro-phenyl)-imidazo[1,2-a]pyrazin-3-yl]-amine hydrochloride,
N-{2-[3-(4-chlorophenoxy)-phenyl]-imidazo[1,2-a]pyridin-3-yl}-N-cyclohexyl-acetamide hydrochloride,
N-cyclohexyl-N-(7-methyl-2-o-tolyl-imidazo[1,2-a]pyridin-3-yl)-acetamide hydrochloride,
N-(2,6-dimethyl-phenyl)-N-[2-(2,4-dimethyl-phenyl)-5-methyl-imidazo[1,2-a]pyridin-3-yl]-acetamide hydrochloride,
1-acetyl-3-(acetyl-cyclohexyl-amino)-7-methyl-2-o-tolyl-imidazo[1,2-a]-pyridin-1-ium) chloride hydrochloride,
cyclohexyl-(2-furan-3-yl-5,7-dimethyl-imidazo[1,2-a]pyridin-3-yl)-amine hydrochloride,
cyclopentyl-(2-furan-3-yl-5,7-dimethyl-imidazo[1,2-a]pyridin-3-yl)-amine hydrochloride,
[2-(4-bromo-2-fluoro-phenyl)-5,7-dimethyl-imidazo[1,2-a]pyridin-3-yl]-cyclopentyl-amine hydrochloride,
cyclopentyl-{5,7-dimethyl-2-[5-(2-nitro-phenyl)-furan-2-yl]-imidazo[1,2-a]-pyridin-3-yl}-amine hydrochloride,
{2-[5-(4-chlorophenyl)-furan-2-yl]-5,7-dimethyl-imidazo[1,2-a]pyridin-3-yl}-cyclopentyl-amine hydrochloride,
cyclopentyl-(2-furan-3-yl-5,7-dimethyl-imidazo[1,2-a]pyridin-3-yl)-amine hydrochloride,
(2-furan-3-yl-5,7-dimethyl-imidazo[1,2-a]pyridin-3-yl)-(11,1,3,3-tetramethylbutyl)-amine hydrochloride,
benzyl-(7-methyl-2-thiophen-3-yl-imidazo[1,2-a]pyridin-3-yl)-amine hydrochloride,
cyclohexyl-(2-furan-3-yl-5,7-dimethyl-imidazo[1,2-a]pyridin-3-yl)-amine hydrochloride,
(2-furan-3-yl-7-methyl-imidazo[1,2-a]pyridin-3-yl)-(1,1,3,3-tetramethylbutyl)-amine hydrochloride,
(5,7-dimethyl-2-thiophen-3-yl-imidazo[1,2-a]pyridin-3-yl)-(1,1,3,3-tetramethyl-butyl)-amine hydrochloride,
[7-ethyl-2-(5-nitro-furan-2-yl)-imidazo[1,2-a]pyridin-3-yl]-(2-methoxybenzyl)-amine,
(2-chlorobenzyl)-[7-ethyl-2-(5-nitro-furan-2-yl)-imidazo[1,2-a]pyridin-3-yl]-amine,
[7-ethyl-2-(5-methyl-furan-2-yl)-imidazo[1,2-a]pyridin-3-yl]-(2-methoxybenzyl)-amine,
(2-chlorobenzyl)-(7-ethyl-2-furan-2-yl-imidazo[1,2-a]pyridin-3-yl)-amine,
(3-chloro-4-fluorophenyl)-[7-ethyl-2-(5-methyl-furan-2-yl)-imidazo[1,2-a]-pyridin-3-yl]-amine,
(2-benzofuran-2-yl-7-ethyl-imidazo[1,2-a]pyridin-3-yl)-(3-chloro-4-fluorophenyl)-amine,
(2-benzofuran-2-yl-7-ethyl-imidazo[1,2-a]pyridin-3-yl)-(3-chlorophenyl)-amine,
(3-chloro-4-fluorophenyl)-{2-[5-(3-chloro-phenyl)-furan-2-yl]-7-ethyl-imidazo[1,2-a]pyridin-3-yl}-amine,
(3-chloro-4-fluorophenyl)-{2-[5-(2-chlorophenyl)-furan-2-yl]-7-ethyl-imidazo[1,2-a]pyridin-3-yl}-amine,
(3-chloro-4-fluorophenyl)-[2-(4,5-dimethyl-furan-2-yl)-7-ethyl-imidazo[1,2-a]pyridin-3-yl]-amine.

13. A method for treating inflammatory pain, or migraine, or both, comprising administering an effective amount of a pharmaceutical composition according to claim 1 to a patient in need thereof.

14. A method according to claim 13, wherein the patient is human.

15. A method for treating inflammatory pain, or migraine, or both, comprising administering an effective amount of a pharmaceutical composition according to claim 6 to a patient in need thereof.

16. A method for treating one or more diseases selected from the group consisting of multiple sclerosis, Parkinson's disease, Alzheimer's disease, Huntington's disease, cerebral ischaemia, diabetes, meningitis, and arteriosclerosis, or a method for healing wounds comprising administering an effective amount of a pharmaceutical composition according to claim 7 to a patient in need thereof.

17. A method according to claim 15, wherein the patient is human.

18. A method for treating one or more diseases selected from the group consisting of multiple sclerosis, Parkinson's disease, Alzheimer's disease, Huntington's disease, cerebral ischaemia, diabetes, meningitis, and arteriosclerosis, or a method for healing wounds, comprising administering an effective amount of a pharmaceutical composition according to claim 12 to a patient in need thereof.
